(19) United States
(12) Patent Application Publication
(10) Pub. No.: US 2022/0401329 A1
(43) Pub. Date: Dec. 22, 2022

(54) ARTIFICIAL MELANIN NANOPARTICLES AND PRECURSOR MOLECULES AS HAIR DYES

(71) Applicant: Northwestern University, Evanston, IL (US)

(72) Inventors: Nathan C. GIANNESCHI, Wilmette, IL (US); Claudia BATTISTELLA, Evanston, IL (US)

(73) Assignee: Northwestern University, Evanston, IL (US)

(21) Appl. No.: 17/775,526

(22) PCT Filed: Oct. 29, 2020
(86) PCT No.: PCT/US20/57939

§ 371 Date: May 9, 2022

Related U.S. Application Data
(60) Provisional application No. 62/935,995, filed on Nov. 15, 2019.

(51) Int. Cl. A61K 8/44; A61K 8/41; A61K 8/66; A61K 8/22; A61K 8/34; A61Q 5/10; A61K 8/02

(52) U.S. Cl. CPC A61K 8/445; A61K 8/415; A61K 8/66; A61K 8/41; A61K 8/22; A61K 8/347; A61Q 5/10; A61K 8/0241; A61K 2800/30; A61K 2800/95; A61K 2800/4324

(57) ABSTRACT

Aspects of the invention include a method of treating hair of a subject with an artificial melanin material comprises contacting in a solution artificial melanin precursors with an oxidizing agent at a temperature greater than or equal to 30° C. in the presence of the hair of the subject to form the artificial melanin material; wherein the artificial melanin material associates with the hair of the subject, thereby treating the hair of the subject. Optionally, the contacting step results in deposition of the artificial melanin material on the hair of the subject and/or uptake of the artificial melanin material into the hair of the subject.

### CROSS-REFERENCE TO RELATED APPLICATIONS

This application claims the benefit of priority to U.S. Provisional Patent Application No. 62/935,995, filed Nov. 15, 2019, which is hereby incorporated by reference in its entirety.

### STATEMENT REGARDING FEDERALLY SPONSORED RESEARCH OR DEVELOPMENT

This invention was made with government support under Award Number AFOSR FA9550-18-1-0142 awarded by the Air Force Office of Scientific Research. The government has certain rights in the invention.

### BACKGROUND OF INVENTION

From countering the effects of aging or illness to expressing one's uniqueness, people have dyed their hair for a myriad of reasons throughout the ages. There are various conventional methods and agents for dying hair, some of which are permanent dyes and others are semi-permanent, for example. Due to such widespread use, hair dye industries are now among the most profitable in the cosmetics sector. Some studies suggest that over 50% of the population in developed countries has dyed their hair at least once in their life. However, conventional methods suffer from requiring long application times, harsh, potentially carcinogenic, reagents, poor persistence, allergic reactions to reagents, and/or poor coloration.

New approaches to hair dying include use of nanoparticles, for example. While the synthesis of gold nanoparticles in human hair has been proposed as an effective way to darken white hair, the long reaction time required by this protocol (e.g., 16 days) hampers its application as an effective hair dyeing method. A much faster approach involves the use of graphene-based sheets for coloration. Hair coated with this material showed good antistatic performance and heat dissipation properties, however, the method was expensive and only produced a single color—black.

Human hair is comprised mainly of protein, at 65-95% by weight. Keratin, the most abundant component, is a group of insoluble protein complexes which impart elasticity, suppleness and resistance to the fibers. Melanin, nature's hair pigment, is mainly distributed in the middle layer of the hair shaft or cortex and is embedded between keratin fibers, where it makes up only 1 to 3% of human hair by weight. These nanometer-scale granular pigments (200-800 nm) generate the naturally beautiful colors found in human hair. Colors arise from the distribution, concentration, and blending of two types of melanin: brown and black eumelanins, and less commonly, red pheomelanins. It follows then that the reduction or disappearance of melanin from hair fibers is the phenomenon that leads to color loss and consequent hair greying and eventually whitening.

Thus, a very interesting approach to darken hair and a desirable alternative to current formulations would be a synthetic version of the naturally occurring nano-sized melanin pigment particles to reestablish color of the hair fibers. However, recent successful human hair dyeing using synthetic melanin required high concentrations of potentially toxic heavy metals such as copper and iron as chelators. Moreover, these demonstrations were limited to a dark brown coloration and lack extensive imaging and characterization of the dyeing mechanism. In other demonstrations of using synthetic melanins, strong oxidative conditions using sodium periodate were employed for successful dopamine deposition on human hair, but this method may not be suitable for widespread use in an at-home or salon application.

It is thus apparent that there is need in the art for new methods and materials for dying hair, which are effective but also address the above challenges associated with conventional approaches, for example by being mild, tunable, biocompatible, metal-free, and long-lasting.

### SUMMARY OF THE INVENTION

Included herein are methods and materials for nontoxic, biocompatible, metal-free, tunable, and long-lasting coloration of human hair. More particularly, provided herein are methods, and associated materials, for efficient formation and deposition of synthetic melanin to human hair without the need for metal chelators or strong oxidants to generate not only black/brown, but also orange/gold colorations from blond hair, for example. For example, according to certain embodiments disclosed herein, different colors can be achieved by tuning reaction conditions such as temperature and solution phase composition. For example, according to certain embodiments disclosed herein, effective hair dyeing can be achieved using milder conditions compared to those conventionally employed for polydopamine coatings. For example, according to certain embodiments disclosed herein, blond and naturally red, brown and grey hair, as well as hair previously dyed with a very bright commercial dye are successfully colored to dark brown/black. These methods, and associated materials, can be used in salons and at home, for example, without degradation of the resulting colored hair and safety of the user of the present materials and methods.

Aspects of the invention include a method of treating hair of a subject with an artificial melanin material comprises contacting in a solution artificial melanin precursors with an oxidizing agent at a temperature greater than or equal to 30° C. in the presence of the hair of the subject to form the artificial melanin material; wherein the artificial melanin material associates with the hair of the subject, thereby treating the hair of the subject.

The present methods may be carried out in a single step or in a series of steps. Optionally, for any method disclosed herein, the artificial melanin precursors are contacted in a solution with an oxidizing agent in the presence of the hair at room temperature and then the temperature is subsequently raised to a temperature equal to or greater than 30° C. to provide for treatment of the hair. Alternatively, the artificial melanin precursors and oxidizing agent are contacted in solution in the presence of the hair at a temperature equal to or greater than 30° C. to provide for treatment of the hair.

Preferably, for any method disclosed herein, the contacting step results in deposition of the artificial melanin material on the hair of the subject and/or uptake of the artificial melanin material into the hair of the subject. Preferably, for any method disclosed herein, the contacting step results in covalent or noncovalent association of the artificial melanin material with the hair of the subject. Optionally, for any method disclosed herein, the contacting step results in noncovalent association of the artificial melanin material with the hair of the subject. Optionally, for any method disclosed herein, the contacting step results in covalent association of the artificial melanin material with the hair of the subject. Preferably, for any method disclosed herein, the contacting step results in a change in the color of the hair of the subject. Preferably, for any method disclosed herein, the change in the color of the hair of the subject remains persistent for at least a period of 5 weeks. Preferably, for any method disclosed herein, the change in the color of the hair of the subject remains persistent for at least a period of one year. Preferably, for any method disclosed herein, the change in the color of the hair of the subject remains persistent for at least 18 washing or rinsing cycles for the hair. Optionally, for any method disclosed herein, the contacting step is carried out for a time selected from the range of 1 minute to 5 hours, optionally 15 minutes to 5 hours, optionally, 30 minutes to 5 hours, optionally 1 hour to 5 hours, optionally 2 hours to 5 hours.

The following describes exemplary embodiments of reaction conditions associated with methods and materials disclosed herein.

Preferably, for any method disclosed herein, the contacting step is carried out in the absence of a metal chelating agent. Optionally, for any method disclosed herein, the contacting step is carried out in the absence of a metal chelating agent. Optionally, for any method disclosed herein, the solution is free of a metal chelating agent. Optionally, for any method disclosed herein, the solution comprises a metal chelating agent having a concentration of less than or equal to 15 mM, optionally less than or equal to 10 mM, optionally less than or equal to 5 mM, optionally less than or equal to 1 mM. Optionally, for any method disclosed herein, the metal chelating agent is an iron-containing chelating agent and/or a copper-containing chelating agent. Preferably, for any method disclosed herein, the contacting step is carried out in the absence of a strong oxidizing condition. Preferably, for any method disclosed herein, the contacting step is carried out in the absence of sodium periodate. Preferably, for any method disclosed herein, the contacting step takes place under conditions providing for polymerization of the artificial melanin precursors to generate the artificial melanin material. Preferably, for any method disclosed herein, the contacting step does not change the mechanical properties of the hair of said subject. Preferably, for any method disclosed herein, the contacting step is carried out at the temperature ranging from 30° C. to 45° C. Preferably, for any method disclosed herein, the contacting step is carried out at physiological temperature. Optionally, for any method disclosed herein, especially but not necessarily any method comprising an enzyme-containing solution, the contacting step is carried out at a pH selected from the range of 4 to 10, optionally 5 to 9, optionally 6 to 8, optionally 6 to 9, optionally 5 to 8, preferably 6.5 to 7.5, preferably 6.7 to 7.3.

Optionally, for any method disclosed herein, especially but not necessarily a method comprising an alkaline solution free of enzymes, the contacting step is carried out at pH greater than 7. Optionally, for any method disclosed herein, especially but not necessarily a method comprising an alkaline solution free of enzymes, the contacting step is carried out at a pH selected from the range of 7 to 12. Optionally, for any method disclosed herein, the solution is an alkaline. Optionally, for any method disclosed herein, the solution is an ammonia solution, or a sodium hydroxide solution. Optionally, for any method disclosed herein, the solution comprises ammonia or sodium hydroxide. Optionally, for any method disclosed herein, the solution is a solution of monoethanolamine or a derivative thereof. Optionally, for any method disclosed herein, the solution comprises a solution of monoethanolamine or a derivative thereof. Optionally, for any method disclosed herein, the solution is an alkaline buffer solution. Optionally, for any method disclosed herein, the solution comprises an alkaline buffer. Optionally, for any method disclosed herein, the buffer solution is a tris(hydroxymethyl)aminomethane buffer solution. Optionally, for any method disclosed herein, the solution is an ammonia solution having a concentration less than or equal to 10% (w/v). Optionally, for any method disclosed herein, the solution comprises ammonia at a concentration less than or equal to 10% (w/v). Optionally, for any method disclosed herein, the solution is an ammonia solution having a concentration selected over the range of 1 to 6% (w/v). Optionally, for any method disclosed herein, the solution comprises ammonia at a concentration selected over the range of 1 to 6% (w/v). Optionally, for any method disclosed herein, the solution is a sodium hydroxide solution having a concentration less than or equal to 0.1 N. Optionally, for any method disclosed herein, the solution comprises sodium hydroxide having a concentration less than or equal to 0.1 N. Optionally, for any method disclosed herein, the solution is a sodium hydroxide solution having a concentration less than or equal to 0.05 N. Optionally, for any method disclosed herein, the solution comprises sodium hydroxide solution at a concentration less than or equal to 0.05 N. Optionally, for any method disclosed herein, the solution is a sodium hydroxide solution having a concentration selected over the range of 0.01 N to 0.1 N. Optionally, for any method disclosed herein, the solution comprises sodium hydroxide at a concentration selected over the range of 0.01 N to 0.1 N. Optionally, for any method disclosed herein, the solution is a sodium hydroxide solution having a concentration selected over the range of 0.05 N to 0.1 N. Optionally, for any method disclosed herein, the solution comprises sodium hydroxide at a concentration selected over the range of 0.05 N to 0.1 N. Optionally, for any method disclosed herein, the solution is a tris(hydroxymethyl)aminomethane solution having a concentration less than or equal to 50 mM. Optionally, for any method disclosed herein, the solution comprises tris(hydroxymethyl)aminomethane at a concentration less than or equal to 50 mM. Optionally, for any method disclosed herein, the solution is a tris(hydroxymethyl)aminomethane solution having a concentration selected over the range of 1 mM to 50 mM. Optionally, for any method disclosed herein, the solution comprises tris(hydroxymethyl)aminomethane at a concentration selected over the range of 1 mM to 50 mM. Optionally, for any method disclosed herein, the solution is a tris(hydroxymethyl)aminomethane solution having a concentration selected over the range of 10 mM to 50 mM. Optionally, for any method disclosed herein, the solution comprises tris(hydroxymethyl)aminomethane at a concentration selected over the range of 10 mM to 50 mM.

Preferably, for any method disclosed herein, the solution is an enzyme containing solution comprising one or more enzymes. Generally, the one or more enzymes facilitate and participate in the formation of the artificial melanin material and/or facilitate the artificial melanin material associating with the hair of the subject. Preferably, for any method disclosed herein, the one or more enzymes comprises one or more oxidoreductase enzymes. Preferably, for any method disclosed herein, the one or more enzymes comprises tyrosinase and/or laccase. Preferably, for any method disclosed herein, the one or more enzymes comprises tyrosinase. Preferably, for any method disclosed herein, the enzyme-containing solution comprises the one or more enzymes at a concentration selected from the range of 1 to 1000 U/mL, optionally 1 to 100 U/mL, optionally 10 to 1000 U/mL, optionally 10 to 100 U/mL. Optionally, for any method disclosed herein, the enzyme-containing solution comprises the one or more enzymes at a concentration selected from the range of 1 nM to 100 μM, optionally 1 nM to 10 μM, optionally 1 nM to 1 μM, optionally 10 nM to 10 μM.

Optionally, for any method disclosed herein, the solution is a solution of monoethanolamine or a derivative thereof having a concentration less than or equal to 6% (w/v). Optionally, for any method disclosed herein, the solution comprises monoethanolamine or a derivative thereof having a concentration less than or equal to 6% (w/v). Optionally, for any method disclosed herein, the solution comprises monoethanolamine or a derivative thereof having a concentration selected over the range of 1% to 3% (w/v). Optionally, for any method disclosed herein, the solution comprises monoethanolamine or a derivative thereof having a concentration selected over the range of 1% to 6% (w/v).

Optionally, for any method disclosed herein, the concentration of the artificial melanin precursors is greater than or equal to 0.1 mg mL⁻¹, optionally greater than or equal to 0.3 mg mL⁻¹, greater than or equal to 0.5 mg mL⁻¹, greater than or equal to 0.8 mg mL⁻¹, greater than or equal to 1 mg mL⁻¹, optionally selected from the range of 0.1 mg mL⁻¹ to 100 mg mL⁻¹, optionally selected from the range of 0.1 mg mL⁻¹ to 50 mg mL⁻¹, optionally selected from the range of 0.1 mg mL⁻¹ to 10 mg mL⁻¹. Optionally, for any method disclosed herein, the concentration of the artificial melanin precursors is selected over the range of 0.1 mg mL⁻¹ to a saturated solution (i.e., a solution saturated with the artificial melanin precursors), optionally 0.3 mg mL⁻¹ to a saturated solution, optionally 0.5 mg mL⁻¹ to a saturated solution, optionally 0.8 mg mL⁻¹ to a saturated solution, optionally 0.8 mg mL⁻¹ to a saturated solution.

Optionally, for any method disclosed herein, the oxidizing agent is O, H₂O₂, O₃, and/or O₂. Optionally, for any method disclosed herein, the oxidizing agent is O₂. Optionally, for any method disclosed herein, the oxidizing agent is present in the ambient atmosphere and the solution is exposed to the ambient atmosphere during the contacting step. Optionally, for any method disclosed herein, the O₂ is from air in contact with the alkaline solution or the enzyme-containing solution, thereby providing a source of O₂ to the solution. Optionally, for any method disclosed herein, the concentration of O₂ in the alkaline solution is greater than 0 and up to a saturated solution (i.e., a solution saturated with O₂). Optionally, for any method disclosed herein, the contacting step is further carried out in the presence of H₂O₂ provided in the alkaline solution. Optionally, for any method disclosed herein, the concentration of the H₂O₂ is less than or equal to 6% (w/v). Optionally, for any method disclosed herein, the concentration of the H₂O₂ is selected over the range of 0.01% to 6% (w/v).

Optionally, for any method disclosed herein, the contacting step is carried out in the absence of one or more metal salts. Optionally, for any method disclosed herein, the contacting step is carried out in the presence of one or more metal salts. Optionally, for any method disclosed herein, the solution is free of metal salts. Optionally, for any method disclosed herein, the solution comprises one or more metal salts. Optionally, for any method disclosed herein, one or more metal salts comprise one or more iron salts and/or one or more copper salts. Optionally, for any method disclosed herein, a concentration of the one or more metals salts in the solution is less than or equal to 15 mM, optionally less than or equal to 10 mM, optionally less than or equal to 5 mM, optionally less than or equal to 1 mM. Optionally, for any method disclosed herein, the contacting step is further carried out in the presence of one or more metal salts and H₂O₂ provided in the solution, for example, in a non-alkaline solution. Optionally, for any method disclosed herein, the one or more metal salts are nontoxic. Optionally, for any method disclosed herein, the metal salt is CuSO₄. Optionally, for any method disclosed herein, the concentration of the CuSO₄ is less than or equal to 15 mM, optionally less than or equal to 10 mM, optionally less than or equal to 5 mM, optionally less than or equal to 1 mM. Optionally, for any method disclosed herein, the concentration of the CuSO₄ is less than or equal to 100 mM. Optionally, for any method disclosed herein, the concentration of the CuSO₄ is selected over the range of 1 mM to 15 mM. Optionally, for any method disclosed herein, the concentration of the CuSO₄ is selected over the range of 1 mM to 100 mM. Optionally, for any method disclosed herein, the concentration of the H₂O₂ is selected over the range of 0.01% to 6% (w/v).

The following describes certain embodiments of artificial melanin precursors and melanin materials.

Optionally, for any method disclosed herein, the artificial melanin precursors are substituted or unsubstituted catechol-based or polyol-based compounds. Optionally, for any method disclosed herein, the artificial melanin precursors are substituted or unsubstituted dopamine monomers. Optionally, for any method disclosed herein, the artificial melanin precursors are substituted or unsubstituted: dopamine monomers, 1,8-Dihydroxynaphthalene or its derivative, tyrosine monomers, tyramine monomers, amino acids, phenolamines, catecholamines, or any combination of these. Optionally, for any method disclosed herein, the artificial melanin precursors are substituted or unsubstituted: dopamine monomers, tyrosine monomers, tyramine monomers, or a combination of these. Optionally, for any method disclosed herein, the artificial melanin precursors are free of phenol derivatives, resorcinol, and/or paraphenylenediamine. Optionally, for any method disclosed herein, the dopamine monomers are selected from the group consisting of substituted or unsubstituted: dihydroxydopamine monomers, dihydroxydopamine dimers, dihydroxydopamine oligomers, dioxydopamine monomers, dioxydopamine dimers, dioxydopamine oligomers, dihydroxynapthalene monomers, dihydroxynapthalene dimers, dihydroxynapthalene oligomers, dioxydopamine monomers, dioxydopamine dimers, dioxydopamine oligomers, and any combination of these. Optionally, for any method disclosed herein, the dopamine monomers are selected from the group consisting of tyrosine and derivatives, phenol and derivatives, resorcinol and derivatives, and any combinations thereof. Optionally, for any method disclosed herein, the dopamine monomers are selected from the group consisting of phenol, resorcinol, L-DOPA, tyrosine and any combinations thereof. Optionally, for any method disclosed herein, the dopamine monomers are selected from the group consisting of cysteine derivatives, chalcogenides derivatives, selenocysteine, and any combinations thereof. Optionally, for any method disclosed herein, the artificial melanin precursors are one or more monomers selected from the group consisting of:

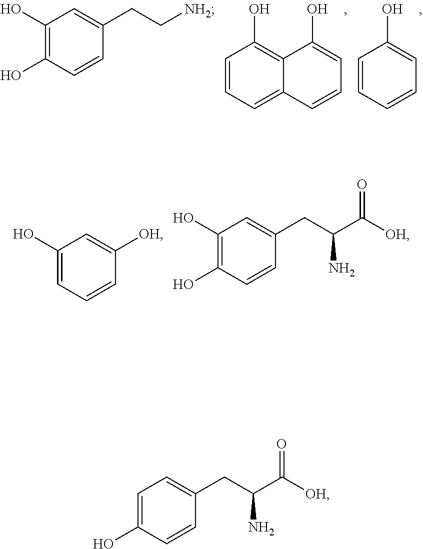

any combinations thereof, and any derivatives thereof. Optionally, for any method disclosed herein, the artificial melanin precursors are one or more monomers having the formula (FX1):

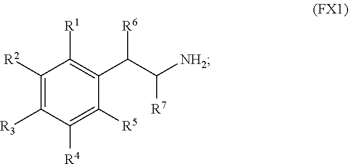

wherein one or more (optionally one, optionally two) of R¹-R⁷ is —OH and wherein each of the other of R¹-R⁷ is a functional group. Optionally, the each of the other of R¹-R⁷ is selected from the group consisting of hydrogen, C₁-C₁₀ alkyl, C₃-C₁₀ cycloalkyl, C₅-C₁₀ aryl, C₅-C₁₀ heteroaryl, acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —NR³⁹R⁴⁰, NR⁴¹COR⁴², C₁-C₁₀ alkyl halide, acrylate, or catechol; wherein each of R³⁰-R⁴² is independently hydrogen, C₁-C₁₀ alkyl or C₅-C₁₀ aryl. Optionally, for any method disclosed herein, the artificial melanin precursors are one or more monomers having the formula (FX2):

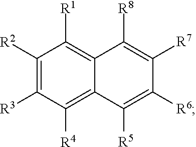

wherein one or more (optionally one, optionally two) of R¹-R⁸ is —OH and wherein each of the other of R¹-R⁸ is a functional group. Optionally, the each of the other of R¹-R⁷ is selected from the group consisting of hydrogen, C₁-C₁₀ alkyl, C₃-C₁₀ cycloalkyl, C₅-C₁₀ aryl, C₅-C₁₀ heteroaryl, acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —NR³⁹R⁴⁰, —NR⁴¹COR⁴², C₁-C₁₀ alkyl halide, acrylate, or catechol; wherein each of R³⁰-R⁴² is independently hydrogen, C₁-C₁₀ alkyl or C₅-C₁₀ aryl. Optionally, for any method disclosed herein, at least a portion of the artificial melanin precursors have one or more thiol-reactive moieties. Optionally, for any method disclosed herein, the thiol-reactive moieties are one or more groups selected from the group consisting of a thiol, maleimide, pyridyl disulfide-based compound, alkene, alkyl halide and any combinations thereof. Optionally, for any method disclosed herein, the artificial melanin material comprises a polymerization product of the artificial melanin precursors. Optionally, for any method disclosed herein, for example, artificial melanin precursors are one or more monomers having the formula (FX1) or (FX2), wherein one or more of R¹-R⁸ is a thiol-reactive moiety, such as a thiol, maleimide, pyridyl disulfide-based compound, alkene, alkyl halide and any combinations thereof.

Preferably, for any method disclosed herein, the artificial melanin material comprises artificial melanin nanoparticles, artificial melanin films, artificial melanin flakes, or any combination of these. Optionally, for any method disclosed herein, the artificial melanin material comprises artificial melanin nanoparticles. Optionally, for any method disclosed herein, the melanin nanoparticles form a coating on the hair, for example, when the contacting step is carried out at temperatures greater than 30° C. in alkaline solution in the presence of air, for example, wherein the oxidant is O₂ in the solution. Optionally, for any method disclosed herein, the coating is characterized by nanostructures having size domains ranging from 5 nm to 500 nm. Optionally, for any method disclosed herein, the coating is characterized by nanostructures having a peak size ranging from 5 nm to 500 nm. Optionally, for any method disclosed herein, the artificial melanin material form a film on the hair, for example, when the contacting step is carried out in the presence of CUSO₄ and H₂O₂.

The following describes various embodiments for treating hair, according to certain embodiments of methods and materials disclosed herein.

Optionally, for any method disclosed herein, the temperature is 35 to 45° C. and: (a) the hair is originally a blond color, the alkaline solution is 1% to 6% (w/v) ammonia solution, wherein upon the contacting step the hair changes to a dark brown color; or (b) the hair is originally a blond color, the alkaline solution is 0.01-0.1 N sodium hydroxide solution, wherein upon the contacting step the hair changes to a dark brown color; or (c) the hair is originally a blond color, wherein the contacting step is further carried out in the presence of CuSO₄ having a concentration selected over the range of 1 mM to 15 mM and H₂O₂ having a concentration of 0.025% to 0.07% (w/v), wherein upon the contacting step the hair changes to a dark brown color; or (d) the hair is originally a blond color, the alkaline solution is 1 to 50 mM tris(hydroxymethyl)aminomethane buffer solution, wherein upon the contacting step the hair changes to a dark grey or light brown color; or (e) the hair is originally a blond color, wherein the contacting step is further carried out in the presence of CuSO₄ having a concentration selected over the range of 1 mM to 15 mM and H₂O₂ having a concentration of 0.025% to 0.07% (w/v), wherein upon the contacting step the hair changes to a dark brown color; or (f) the hair is originally a blond color, wherein the contacting step is further carried out in the presence of CuSO₄ having a concentration selected over the range of 1 mM to 15 mM and H₂O₂ having a concentration of 0.1% to 0.2% (w/v), wherein upon the contacting step the hair changes to a dark brown color; or (g) the hair is originally a blond color, wherein the contacting step is further carried out in the presence of CuSO₄ having a concentration selected over the range of 1 mM to 15 mM and H₂O₂ having a concentration of 0.2% to 0.4% (w/v), wherein upon the contacting step the hair changes to a brown color with shades of red; or (h) the hair is originally a blond color, wherein the contacting step is further carried out in the presence of CuSO₄ having a concentration selected over the range of 1 mM to 15 mM and H₂O₂ having a concentration of 2% to 4% (w/v), wherein upon the contacting step the hair changes to an orange color; (i) the hair is originally a blond color, the alkaline solution is 1% to 4% (w/v) ammonia solution and wherein the contacting step is further carried out in the presence of H₂O₂ having a concentration of 0.1% to 0.2% (w/v), wherein upon the contacting step the hair changes to a brown color; or (j) the hair is originally a blond color, the alkaline solution is 1% to 4% (w/v) ammonia solution and wherein the contacting step is further carried out in the presence of H₂O₂ having a concentration of 0.2% to 0.4% (w/v), wherein upon the contacting step the hair changes to a brown color with orange shades; or (k) the hair is originally a blond color, the alkaline solution is 1 to 4% (w/v) ammonia solution and wherein the contacting step is further carried out in the presence of H₂O₂ having a concentration of 2% to 4% (w/v), wherein upon the contacting step the hair changes to a bright blond color; or (I) the hair is originally a brown color, the alkaline solution is 1% to 3% (w/v) ammonia solution, wherein upon the contacting step the hair changes to a dark brown color; or (m) the hair is originally a red color, the alkaline solution is 1% to 3% (w/v) ammonia solution, wherein upon the contacting step the hair changes to a dark brown color with shades of red; or (n) the hair is originally a grey color, the alkaline solution is 1% to 3% (w/v) ammonia solution, wherein upon the contacting step the hair changes to a dark brown color; or (o) the hair is originally a grey color, the alkaline solution is 1% to 3% by wt ammonia solution and wherein the contacting step is further carried out in the presence of H₂O₂ having a concentration of 0.1% to 0.2% (w/v), wherein upon the contacting step the hair changes to a dark brown color with shades of red.

According to certain embodiments, the methods can be described as including in situ formation of artificial melanin materials and deposition on to the hair and/or update into the hair. Optionally, for any method disclosed herein, a method of treating hair of a subject with an artificial melanin material comprises: contacting in an alkaline solution artificial melanin precursors with an oxidizing agent at a temperature greater than or equal to 30° C. in the presence of the hair of the subject to form the artificial melanin material; wherein at least a portion of the artificial melanin precursors have one or more thiol-reactive moieties; and wherein the artificial melanin material associates with the hair of the subject, thereby treating the hair of the subject. Optionally, for any method disclosed herein, for example, the method is carried out in 2 steps: (i) first step, mixing the hair with the artificial melanin precursors (and other solution components) at room temperature and (ii) second step—raising the temperature so as to provide for oxidation and formation of the artificial melanin materials at the higher temperature.

According to certain embodiments, the methods can be described as including ex situ formation and deposition. Optionally, for any method disclosed herein, a method of treating hair of a subject with an artificial melanin material comprises: contacting in an alkaline solution the hair of the subject with the artificial melanin material having thiol-reactive moieties; wherein the artificial melanin material associates with the hair of the subject, thereby treating the hair of the subject. Optionally, for any method disclosed herein, the thiol-reactive moieties are one or more groups selected from the group consisting of a thiol, maleimide, pyridyl disulfide-based compound, alkene, alkyl halide and any combinations thereof.

Optionally, for any method disclosed herein, the contacting step is carried out in the absence of a metal chelating agent. Optionally, for any method disclosed herein, the contacting step is carried out in the absence of a strong oxidizing condition. Optionally, for any method disclosed herein, the contacting step is carried out in the absence of sodium periodate.

A variety of materials are disclosed herein for dying hair, which are compatible with methods disclosed herein. Aspects of the invention include a composition of matter comprises the hair of the subject treated with the artificial melanin material generated by any of the methods.

Aspects of the invention include a composition of matter comprises hair of a subject having a coating of artificial melanin nanoparticles, wherein the coating of artificial melanin nanoparticles is characterized by nanostructures having size domains ranging from 5 nm to 500 nm; wherein the artificial melanin nanoparticles associate with the hair of the subject.

Aspects of the invention include an artificial melanin material comprises a polymerization product of artificial melanin precursors at least a portion of which having one or more thiol-reactive moieties.

Optionally, an artificial melanin material is produced by a method comprising: contacting in an alkaline solution artificial melanin precursors with an oxidizing agent at a temperature greater than or equal to 18° C. in the presence of the hair of the subject to form the artificial melanin material; wherein the artificial melanin material associates with the hair of the subject, thereby treating the hair of the subject.

Optionally, an artificial melanin material is produced by a method comprising: contacting in an alkaline solution the hair of the subject with the artificial melanin material having thiol-reactive moieties; wherein the artificial melanin material associates with the hair of the subject, thereby treating the hair of the subject.

Preferably, in any method, composition, formulation, and material disclosed herein, the thiol-reactive moieties are one or more groups selected from the group consisting of a thiol, maleimide, pyridyl disulfide-based compound, alkene, alkyl halide and any combinations thereof.

Preferably, in any method, composition, formulation, and material disclosed herein, the artificial melanin material comprises artificial melanin nanoparticles. Preferably, in any method, composition, formulation, and material disclosed herein, the solution is free of artificial melanin precursors or monomers.

Aspects of the invention also include a method of treating hair of a subject with an artificial melanin material, the method comprising: contacting in an enzyme-containing solution the hair of said subject with the artificial melanin material having one or more thiol-reactive moieties; wherein the enzyme-containing solution comprises one or more enzymes; and wherein said artificial melanin material associates with said hair of said subject, thereby treating the hair of said subject. Optionally, the one or more thiol-reactive moieties are one or more groups selected from the group consisting of a thiol, maleimide, pyridyl disulfide-based compound, alkene, alkyl halide and any combinations thereof. Preferably, the one or more enzymes comprises one or more oxidoreductase enzymes. Preferably, the one or more enzymes comprises tyrosinase and/or laccase. Optionally, the contacting step is carried out in the absence of a metal chelating agent. Optionally, the said contacting step is carried out in the absence of a strong oxidizing condition. Optionally, the contacting step is carried out in the absence of sodium periodate. Optionally, the contacting step does not change the mechanical properties of the hair of said subject. Preferably, the artificial melanin material comprises artificial melanin nanoparticles. Optionally, the solution is free of artificial melanin precursors or monomers. Preferably, the solution has a pH selected from the range of 4 to 10.

According to certain embodiments, methods disclosed herein include treatment of hair under room temperature conditions. Aspects of the invention include a method of treating hair of a subject with an artificial melanin material comprises: contacting in a solution artificial melanin precursors with an oxidizing agent at a temperature greater than or equal to 18° C. in the presence of the hair of the subject to form the artificial melanin material; wherein the artificial melanin material associates with the hair of the subject, thereby treating the hair of the subject; wherein the hair is originally a blond color, wherein the contacting step is further carried out in the presence of CuSO₄ having a concentration selected over the range of 1 mM to 15 mM and H₂O₂ having a concentration of 0.025% to 0.07% by weight, wherein upon the contacting step the hair changes to a dark brown color; or the hair is originally a blond color, wherein the contacting step is further carried out in the presence of CuSO₄ having a concentration selected over the range of 1 mM to 15 mM and H₂O₂ having a concentration of 0.1% to 0.2% by weight, wherein upon the contacting step the hair changes to a dark brown color; or the hair is originally a blond color, wherein the contacting step is further carried out in the presence of CuSO₄ having a concentration selected over the range of 1 mM to 15 mM and H₂O₂ having a concentration of 0.2% to 0.4% by weight, wherein upon the contacting step the hair changes to a brown color with shades of red; or the hair is originally a blond color, wherein the contacting step is further carried out in the presence of CuSO₄ having a concentration selected over the range of 1 mM to 15 mM and H₂O₂ having a concentration of 2% to 4% by weight, wherein upon the contacting step the hair changes to a dark orange color; the hair is originally a blond color, the alkaline solution is 1% to 4% by wt ammonia solution and wherein the contacting step is further carried out in the presence of H₂O₂ having a concentration of 0.1% to 0.2% by weight, wherein upon the contacting step the hair changes to a brown color; or the hair is originally a blond color, the alkaline solution is 1% to 4% by wt ammonia solution and wherein the contacting step is further carried out in the presence of H₂O₂ having a concentration of 0.2% to 0.4% by weight, wherein upon the contacting step the hair changes to a brown color with orange shades; or the hair is originally a blond color, the alkaline solution is 1% to 4% by wt ammonia solution and wherein the contacting step is further carried out in the presence of H₂O₂ having a concentration of 2% to 4% by weight, wherein upon the contacting step the hair changes to a bright blond color.

Aspects of the invention include a method, composition and/or material is provided for changing the color of the hair of said subject. Aspects of the invention include a method, composition and/or material for darkening the color of the hair of said subject. Aspects of the invention include a method, composition and/or material for restoring the color of the hair of said subject to its natural color. Aspects of the invention include a method, composition and/or material for the coloring of the eyebrows of said subject. Aspects of the invention also include a solution for treating hair or changing color of hair, the solution being according to any embodiment or any combination of embodiments disclosed herein. Aspects of the invention also include a formulation for treating hair or changing color of hair, the formulation comprising a solution according to any embodiment or any combination of embodiments disclosed herein.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1C. Oxidation of dopamine (DA) to polydopamine (PDA). (FIG. 1A) Oxidative polymerization of DA to PDA and (FIG. 1B) resulting dark brown/black synthetic melanin nanoparticles as determined by (FIG. 1C) scanning electron microscopy (SEM) imaging.

FIGS. 2A-2D. Higher temperature enhances polydopamine deposition and hair darkening. (FIGS. 2A-2B) Photographs of hair samples before (1) and after dyeing with polydopamine using 3% NH₄OH (2), 0.05 N NaOH (3), 10 mM CuSO_{4/15} mM H₂O₂ (4), Tris buffer pH 8.5 (5), as well as color of the resulting polydopamine solutions. Dopamine polymerizations were carried out for 2 hours at (FIG. 2A) room temperature and (FIG. 2B) 37-40° C. FIG. 2C. Optical microscopy images of the hair samples dyed at 37-40° C. are also reported. FIG. 2D. UV-Vis spectra of solutions obtained after dopamine polymerization using 3% NH₄OH and 0.05 N NaOH at either room temperature (8 hours reaction) or at 37-40° C. (2 and 5 hours reaction). In every case, hair samples were washed 5 times with water and 3 times with a 10% shampoo solution prior to imaging/analysis (see Materials and Methods for protocols and for hair sample descriptions from alternative vendors).

FIGS. 3A-3D. Hair color can be tuned by changing temperature and NH₄OH concentration. FIG. 3A. Hair darkening using NH₄OH at room temperature (R.T., 3% NH₄OH) and at 37-40° C. (3% and 6% NH₄OH) as compared to untreated blond hair (1). FIG. 3B. Image analysis (color intensity) of photograph (FIG. 3A), showing hair darkening, from blond (1), to dark blond (2), and dark brown (3,4). FIG. 3C. Comparison between the color of untreated blond hair (1), hair dyed using 3% NH₄OH (2), 6% NH₄OH (3), as well as hair dyed using a commercial dark brown hair dye (4) and naturally pigmented dark brown hair (5). FIG. 3D. Corresponding description of optical micrographs of individual hair fibers. RGB color analysis of hair samples in photograph (FIG. 3A) are included in FIG. 21.

FIGS. 4A-4G. Effect of H₂O₂ and temperature on hair shades from red, to dark or light gold hues. FIG. 4A. Photograph of blond hair and hair dyed with polydopamine using (FIG. 4B) 10 mM CuSO_{4/1} M H₂O₂ at room temperature (2) and at 37-40° C. (3). FIGS. 4C-4D. Effect of H₂O₂ (50 mM (4,7), 100 mM (5,8) and 1 M (6,9)) on hair dyeing with polydopamine and 3% NH₄OH solutions. The reactions were performed at (FIG. 4C) 37-40° C. (4,5,6) and at (FIG. 4D) room temperature (7,8,9). FIG. 4E. Photograph of natural red hair (10). Optical images of the corresponding single hair fibers are shown in the lower panel. FIG. 4F. Image analysis (color intensity) and (FIG. 4G) RGB color ratios of photographs of hair 1-6. Description of color analysis of samples dyed at room temperature (7,8,9) are included in FIG. 21. All dyeing reactions were carried out for 2 hours and hair samples were washed 5 times with water and 3 times with a 10% shampoo solution prior to imaging/analysis.

FIGS. 5A-5D. Retention of hair color after 18 washes. FIG. 5A. Photographs of hair dyed with polydopamine using 6% NH₄OH for 2 hours at 37-40° C. before and after 18 washes with a 10% shampoo solution and (FIG. 5B) color intensity analysis. FIG. 5C. Comparison between the color of the soiled solution from synthetic melanin-dyed hair and hair dyed using a commercial dark brown hair dye. FIG. 5D. RGB color ratios (FIG. 5A) of hair before and after washes.

FIGS. 6A-6D. Oxidation in alkaline conditions results in PDA nanoparticles coating the hair surface as determined by SEM, optical microscopy and TEM imaging. FIG. 6A. SEM images of untreated blond hair, natural dark brown hair and blond hair, dyed with dopamine (3% NH₄OH and 0.05 N NaOH), scale bars 25 μm (top row) and 2 μm (second row). FIG. 6B. Optical microscopy (scale bars 40 μm) and (FIG. 6C) TEM images of hair cross-sections. Black arrows and (FIG. 6D) higher magnification TEM images highlight the PDA coatings obtained using NH₄OH (1) and NaOH (2) as oxidants.

FIGS. 7A-7C. Polydopamine deposition is compatible with a wide range of hair substrates. FIG. 7A. Natural virgin red (1) and brown (3) hair and (FIG. 7B) hair dyed with a purple hair dye (5) were used as hair substrates. Dopamine oxidation was carried out using 3% NH₄OH as shown in FIGS. 2A-2D. FIG. 7C. Virgin grey hair (7) was dyed using 3% NH₄OH (8) and 3% NH₄OH/100 mM H₂O₂ (as for sample 5 in FIGS. 4A-4G). In every case, reactions were carried out at 37-40° C. for 2 hours and hair samples were washed 5 times with water and 3 times with a 10% shampoo solution prior to imaging/analysis.

FIGS. 8A-8C. Blond, human hair purchased from a second vendor (Emosa) was dyed using dopamine formulations (companion to FIG. 1, main text). A) Photographs of hair before (1) and after dyeing at room temperature for 2 hours using 3% NH₄OH (2), 0.05 N NaOH (3), 10 mM CuSO_{4/1} M H₂O₂ (4) and Tris buffer pH 8.5 (5). B), as for A), but the dyeing was conducted at 37-40° C. C) Optical micrographs of individual hair fibers dyed at 37-40° C. (panel B).

FIGS. 9A-9D. UV-Vis spectra of polydopamine solutions obtained at different reaction times using (FIG. 9A) Tris buffer pH 8.5, (FIG. 9B) 3% NH₄OH, (FIG. 9C) 0.05 N NaOH, and (FIG. 9D) 10 mM CuSO_{4/1} M H₂O₂. Reactions were carried out at room temperature (left panels) and at T=37-40° C. (right panels).

FIGS. 10A-100. Color of polydopamine solutions obtained using different reaction conditions. Left to right in each panel: Tris buffer pH 8.5, 3% NH₄OH, 0.05 N NaOH, 10 mM CuSO_{4/1} M H₂O₂ and 10 mM CuSO_{4/15} mM H₂O₂). FIGS. 10A-10B. Reactions were carried out at room temperature. FIGS. 10C-10D. 37-40° C. (FIGS. 10A and 10C) Imaged after 20 mins. (FIGS. 10B and 10D) Imaged after 2 hrs.

FIG. 11A. RGB color ratios. 1) Untreated blond hair. 2) Dopamine, 3% NH₄OH at room temperature. 3) Dopamine, 3% NH₄OH and 4) Dopamine, 6% NH₄OH at 37-40° C. FIG. 11B. Comparison between RGB color ratios of hair samples 1-4. The photographs of samples 1-4 are shown in FIG. 3A.

FIGS. 12A-12D. Photographs of hair before (1) and after dyeing with dopamine using 3% NH₄OH (2), 0.05 N NaOH (3), 10 mM CuSO_{4/1} M H₂O₂ (4) and Tris buffer pH 8.5 (5). Dyeing was carried out for 2 hours at (FIG. 12A) room temperature and (FIG. 12B) 37-40° C. The colors of the resulting polydopamine solutions in which the hair was suspended, are shown in vials. FIG. 12C. Optical microscopy images of an untreated blond hair fiber (sample 1 in FIGS. 12A-12B) and (FIG. 12D) hair dyed using 10 mM CuSO_{4/1} M H₂O₂ at 37-40° C. (Sample 4 in FIG. 12B).

FIGS. 13A-13B. Photographs of hair dyed with polydopamine and CuSO₄/H₂O₂ using increasing H₂O₂ concentrations: 15 mM (1), 50 mM (2), 100 mM (3) and 1 M H₂O₂ (4). Reactions were carried out at (FIG. 13A) room temperature and (FIG. 13B) 37-40° C.

FIG. 14A. RGB color analysis of hair samples (1 to 6) highlights different RGB components for the untreated blond hair sample (1) and for hair dyed with dopamine solutions. Reaction conditions: 10 mM CuSO_{4/1} M H₂O₂ at room temperature (2) and at 37-40° C. (3) as well as 3% NH₄OH/50 mM, 100 mM or 1 M H₂O₂ at 37-40° C. (4,5,6).

FIG. 14B. RGB color analysis of hair dyed using 3% NH₄OH/50 mM, 100 mM or 1 M H₂O₂ at room temperature (7,8,9). FIG. 14C. RGB color ratios and (FIG. 14D) color intensities of hair samples 7-9 are compared to those of untreated blond hair (1) and hair dyed using 10 mM CuSO_{4/1}M H₂O₂, either at room temperature (2) or at 37-40° C. (3). Photographs of hair samples 1-9 are shown in FIGS. 4A-4G.

FIG. 15. RGB color ratios of hair dyed with polydopamine using 6% NH₄OH for 2 hours at 37-40° C. before (1) and after (2) 18 washes with a 10% shampoo solution. A photograph is shown in FIG. 3A.

FIG. 16A. Photograph, (FIG. 16B) RGB color analysis and (FIG. 16C) comparison between RGB color ratios and (FIG. 16D) color intensities of hair samples dyed with a commercially available hair dye before and after 18 washes with 10% shampoo. FIG. 16E is a plot characterizing color intensity for the two samples (hair samples dyed with a commercially available hair dye before and after 18 washes with 10% shampoo.

FIG. 17A. FTIR-ATR spectra of hair before (dashed line) and after (solid lines) dyeing with dopamine formulations (a=Tris buffer pH 8.5, b=3% NH₄OH, c=0.05 N NaOH, d=10 mM CuSO_{4/1} M H₂O₂) or with a dark brown, commercial dye (Dyed). FIG. 17B. Hair dyed using the same protocols as FIG. 17A but derived from a second vendor (Emosa) were also analyzed and showed very similar results. FIG. 17C. FTIR-ATR spectra of lyophilized polydopamine nanoparticles.

FIGS. 18A-180. SEM images of polydopamine solutions obtained after a 2 h reaction at room temperature (left images) and at 37-40° C. (center and right images). Reaction conditions: (FIG. 18A) Tris buffer pH 8.5, (FIG. 18B) 3% NH₄OH, (FIG. 18C) 0.05 N NaOH, and (FIG. 18D) 10 mM CuSO_{4/1} M H₂O₂. Purified polydopamine nanoparticles obtained after 2 h oxidation at 37-40° C. using alkaline conditions (FIGS. 18A-18C) are shown in the right panel. Reaction solutions obtained using (FIG. 18D) CuSO₄/H₂O₂ did not result in nanoparticles formation, one example of SEM image is reported in the center panel (FIG. 18D, 37-40° C.).

FIG. 19A. SEM images of hair purchased from Jerome Krause Fashion Hair and dyed using dopamine and 10 mM CuSO_{4/1}M H₂O₂ and (FIG. 19B) hair dyed with a commercial dark brown hair dye. FIG. 19C. SEM images of hair samples purchased from Emosa and (FIG. 19D) dyed with a commercial dark brown hair dye or (FIG. 19E) treated with 3% NH₄OH. Polydopamine deposition was carried out at 37-40° C. for 2 h using (FIG. 19F) 3% NH₄OH, (FIG. 19G) 0.05 N NaOH, (FIG. 19H) Tris buffer pH 8.5 and (FIG. 19I) 10 mM CuSO_{4/1} M H₂O. Scale bars 25 μm (left columns), and 2 μm (right columns).

FIG. 20. Shear and stress curves of hair before (untreated) and after dopamine dyeing using 3% and 6% NH₄OH, as well as after dyeing with a commercial dark brown hair dye (Dyed). Results are reported as the average of five independent experiments.

FIG. 21 provides a Table summarizing the starting materials and resulting hair color for a range of hair treatment methods and conditions.

FIGS. 22A-22D. Melanogenesis: Tyrosinase-mediated Oxidation of L-Tyrosine to Eumelanin. FIG. 22A. Mechanism of chemoenzymatic oxidation of tyrosine (L-Tyr) to eumelanin occurring inside melanocytes (FIG. 22B). FIG. 22C. TEM images of hair cross-section shows melanosomes (indicated by black arrows) in the hair shaft. Scale bar=2.5 μm. FIG. 22D. Structure of dopamine (DA) and tyramine (TA) synthetic melanin precursors investigated in this study together with A) L-Tyr.

FIGS. 23A-23D. Optimization of the Reaction Parameters to Enhance the Kinetics of the Chemoenzymatic Polymerization. FIG. 23A. Photographs of vials containing 2.8 mM L-Tyr, 3.6 mM TA and 2.6 mM DA (0.5 mg/mL in water) treated with 84 nM tyrosinase as a function of reaction time (10 min, 30 min, 1 h, 2 h, 6 h) at rt and at 35° C. Reactions performed in DPBS yielded comparable results (Supporting Information). FIG. 23B. UV-Vis spectra of the supernatant of L-Tyr chemoenzymatic oxidation reactions carried out at rt (top panel) and at 35° C. (bottom panel). FIG. 23C. RP-HPLC curves show the L-Tyr consumption after 2 h reaction at either rt or 35° C. FIG. 23D. RP-HPLC analysis of reaction solutions performed at 35° C. for 2 h varying tyrosinase concentrations (84 nM and 420 nM, see also Example 3 discussion). UV-Vis spectra and RP-HPLC traces of TA and DA chemoenzymatic oxidation reactions are reported in FIGS. 30A-30D and 31A-31B.

FIGS. 24A-24F. Optimized Chemoenzymatic Reaction Affords Hair Colors Comparable to those Obtained via Basic Oxidation. Color of hair (sample 1) dyed via tyrosinase-mediated oxidation of 2.8 mM L-Tyr (sample 2), 3.6 mM TA (sample 3) or 2.6 mM DA (sample 4) for 2 h at (FIG. 24A) rt and (FIG. 24B) 35° C. FIG. 24C. Color of hair dyed using 36 mM TA or 26 mM DA and 850 nM tyrosinase for 2 h at rt and (FIG. 24D) at 35° C. Hair dyed using base-mediated oxidation (3% aq. NH₄OH) of DA to PDA was used as a control (sample 5). FIG. 24E. Color of the solution after a 2 h reaction and (FIG. 24F) optical micrographs of hair dyed via enzymatic oxidation of L-Tyr (sample 2), TA (sample 3), DA (sample 4). Both (FIG. 24E) solutions and (FIG. 24F) coated hair obtained via basic oxidative polymerization of DA (sample 5) were also imaged as a comparison. For all studies, 2.8 mM L-Tyr was used due to its limited solubility in water.

FIGS. 25A-25E. Chemoenzymatic Reaction Yields Dark Hair Color while Minimizing Hair Penetration and Potential Damage. FIG. 25A. Photograph of uncoated blond hair (BL, sample 1), natural brown hair (BR, sample 2), hair dyed via basic oxidative polymerization of DA (sample 3), hair dyed via chemoenzymatic oxidative polymerization of DA (sample 4), TA (sample 5) and L-Tyr (sample 6). Photograph of hair dyed with a commercial dark brown hair dye is reported in the Supporting Information. FIG. 25B. Reflectance spectra of uncoated blond (sample 1) and brown (2) hair, as well as blond hair coated (samples 3-6, enlarged in the right panel) via basic (sample 3) and chemoenzymatic (sample 4-6) polymerization. The colored lines represent the mean reflectance, and the colored areas represent the standard deviation. FIG. 25C. Mean brightness of hair samples 1-6. All optical measurements were performed in triplicate. FIG. 25D. Optical micrographs of blond and brown hair cross sections as well as hair previously coated via either basic or chemoenzymatic oxidative polymerization. Scale bars 50 μm. Images as well as reflectance spectra and mean brightness of hair dyed with a commercial dark brown dye are reported in the Supporting Information. FIG. 25E. SEM images of hair incubated with either 3% aq. NH₄OH or with the enzyme tyrosinase in the absence of monomer for 2 h at 35° C. Black arrows indicate cuticles lifting in chemically treated hair. Scale bars: 50 μm and 10 μm. Additional images, including those of untreated hair, are shown elsewhere herein.

FIGS. 26A-26B. Basic and Chemoenzymatic Oxidative Polymerizations Result in the Formation of Nanoparticles in Solution. FIG. 26A. Dynamic Light Scattering (DLS) of nanoparticles obtained from spontaneous DA oxidation in basic environment (3% aq. NH₄OH), and from the chemoenzymatic oxidation of DA, TA and L-Tyr. Reactions were carried out for 2 h, at 35° C. using 36 mM TA or 26 nM DA in water. 2.8 mM L-Tyr was used due to its limited solubility in water. FIG. 26B. SEM images of the nanoparticles contained in the drop casted solutions. Scale bars=2 μm.

FIGS. 27A-27D. Synthetic Melanin Deposition onto Human Hair can be Achieved via Both Basic- and Enzymatically-triggered Deposition. FIG. 27A. SEM images and tapping mode AFM of hair coated via basic oxidative polymerization of DA and (FIG. 27B) via chemoenzymatic oxidative polymerization of DA, TA and L-Tyr. Reactions were carried out for 2 h, at 35° C. using either 36 mM TA, 26 mM DA or 2.8 mM L-Tyr. The red line in the images indicates the profiles along the fast scan axis, which are compared to that of uncoated hair in the bottom scan line in FIG. 27C. FIG. 27D. AFM and SEM images of uncoated hair. More AFM images are included in the Supporting Information. AFM scale bars=4 μm and image width 20 μm. SEM scale bars=2.5 μm.

FIGS. 28A-28D. Synthetic Melanin Coatings on the Hair Surface Protect Inner Hair Cuticles from Photodamage. FIG. 28A. Photograph of blond (BL) and brown (BR) hair samples, as well as hair (blond) after coating via basic oxidative polymerization of DA or by chemoenzymatic oxidative polymerization of DA and TA before and after 10 days of UVA and UVB irradiation. FIG. 28B. Example of SEM images of uncoated hair after 10 days of UVA (top) and UVB (bottom) irradiation. Scale bars 5 μm. Black arrows indicate hair photodamage occurring via hole formation and cuticle damaging/lifting. More images are reported in the Supporting Information. FIG. 28C. Reflectance spectra of hair coated via basic oxidation of DA as well as chemoenzymatic oxidation of both DA and TA before and after UVA and UVB irradiation. The colored lines represent the mean reflectance, and the colored areas represent the standard deviation. The spectra of uncoated hair and hair coated via basic oxidative polymerization are reported in the Supporting Information. All optical measurements were performed in triplicate. FIG. 28D. Optical microscopy images of uncoated and coated hair cross-sections before and after 10 days of UVA and UVB irradiation. Black arrows highlight cuticle exfoliation and uncoated hair damage. All images were acquired using the same settings and magnification. Scale bars=50 μm.

FIG. 29. Color of 2.8 mM L-tyrosine (L-Tyr), 3.6 mM tyramine (TA) and 2.6 mM dopamine (DA) (0.5 mg/mL in DPBS) as a function of reaction time. Chemoenzymatic oxidative polymerizations were carried out at room temperature (rt) or at 35° C. using 84 nM tyrosinase.

FIGS. 30A-30D. UV-Vis spectra of the supernatant of chemoenzymatic oxidation polymerizations at different reaction times. FIGS. 30A-30B. TA and (FIGS. 30C-30D) DA (0.5 mg/mL in DPBS). The oxidations were carried out at (FIGS. 30A and 30C) rt and (FIGS. 30B and 30D) 35° C., using 84 nM tyrosinase in water.

FIG. 31A. Consumption of TA and (FIG. 31B) DA monomers from the reaction solution as monitored by RP-HPLC. Chemoenzymatic oxidations were carried out at both rt and at 35° C., for 2 h using 84 nM tyrosinase. Reactions were performed using 3.6 mM TA and 2.6 mM DA (0.5 mg/mL monomer) in water.

FIG. 32A. Optical microscopy images of an uncoated blond hair fiber and hair dyed via chemoenzymatic oxidative polymerization of 2.8 mM L-Tyr, 3.6 mM TA and 2.6 mM DA (0.5 mg/mL) at 35° C. for 2 h using tyrosinase (84 nM). FIG. 32B. Optical microscopy images of hair dyed using 36 mM TA and 26 mM DA (5 mg/mL) at room temperature for 2 h using tyrosinase (850 nM). L-Tyr concentration was limited to 2.8 mM (0.5 mg/mL) due to its lower solubility in water. Scale bar 100 μm.

FIG. 33A. L-Tyrosine-OMe (L-Tyr-OMe) consumption as determined by RP-HPLC. The oxidation reaction was carried out for 2 hours at 35° C. 0.5 mg/mL monomer concentration and tyrosinase 84 nM were used for comparison to the other previously screened monomers. FIG. 33B. Color of 0.5 mg/mL (1) and 5 mg/mL L-Tyr-OMe (2) solutions oxidized using 84 nM as well as five-fold (840 nM) tyrosinase, respectively. FIG. 33C. Color of hair dyed using L-Tyr-OMe, 5 mg/mL at 35° C. for 2 hours using 840 nM tyrosinase (untreated hair samples are shown in FIGS. 23A-23D).

FIG. 34A. Photograph of hair dyed with a commercial dark brown dye and (FIG. 34B) optical micrograph of hair cross section, scale bar 50 μm. FIG. 34C. Reflectance spectra of dyed hair, the colored line represents the mean reflectance, and the colored area represents the standard deviation. FIG. 34D. Comparison between mean brightness of untreated blond hair (yellow) and dyed hair (blue). Optical measurements were performed in triplicate.

FIG. 35A. SEM images of untreated hair and (FIG. 35B.) hair incubated at 35° C. in the presence of tyrosinase for 2 hours. Scale bars: 50 μm, 20 μm and 10 μm (top to bottom).

FIG. 36. SEM images of hair incubated at 35° C. in 3% aq. NH₄OH solution for 2 hours. Scale bars: 50 μm, 20 μm and 10 μm (top to bottom).

FIGS. 37A-37J. SEM images of nanoparticles obtained via (FIG. 37A) spontaneous DA oxidation in basic environment (3% aq. NH₄OH) and via (FIG. 37B) DA, (FIG. 37C) TA and (FIG. 37D) L-Tyr chemoenzymatic oxidations. Reactions were carried out for 2 hours, at 35° C. using 2.8 mM (0.5 mg/mL) L-Tyr (84 nM tyrosinase) and 36 mM TA or 26 mM DA (5 mg/mL) in water (840 nM tyrosinase). Scale bars: 5 μm. The corresponding TEM images are also included in the bottom panel (FIGS. 37E-37H). Scale bars 300 nm. FIG. 37I. Zeta potential plots and (FIG. 37J) values for the nanoparticle solutions in water.

FIG. 38A. SEM images of hair coated with the nanoparticles via DA basic oxidative polymerization (3% aq. NH₄OH) and via chemoenzymatic oxidative polymerization of (FIG. 38B) DA, (FIG. 38C) TA and (FIG. 38D) L-Tyr. Scale bars=30 μm.

FIG. 39A. Optical micrograph of Bruker RTesp-150 cantilever (nominal spring constant 5 N/m and tip radius 8 nm) scanning a strand of hair (sample 5). FIG. 39B. Tapping mode AFM image in air of uncoated hair (1) and hair coated via basic oxidative polymerization of DA (2), and via chemoenzymatic oxidative polymerization of DA (3), TA (4) and L-Tyr (5). The red/black lines across the images indicates the profile which is plotted in FIG. 39C. Scale bars 1 μm.

FIG. 40. SEM images of brown and blond hair after 10 days of UVA and UVB irradiation. Hair sprayed with a commercial hair sunscreen prior treatment was also imaged (Sunscreen). Scale bars: 50 μm, 10 μm and 10 μm (from top to bottom).

FIG. 41A. Reflectance spectra of uncoated blond and brown hair before and after 10 days UVA and UVB irradiation. The colored lines represent the mean reflectance, and the colored areas represent the standard deviation for each spectral band. FIG. 41B. Mean brightness of uncoated blond (1) and brown (2) hair, as well as hair coated via basic oxidative polymerization of DA (3) and chemoenzymatic oxidative polymerization of DA (4) and TA (5), before and after 10 days UVA and UVB irradiation.

FIG. 42. Optical microscopy images of cross sections of uncoated blond hair (with and without sunscreen treatment) before and after UVA and UVB irradiation. Sunscreen was applied once before UV irradiation. All settings, including magnification were kept constant and at least 4 images per irradiated sample were acquired. Higher magnification images of some damaged hair examples are shown in the bottom panel. Scale bars: 50 μm.

FIG. 43. Optical microscopy images of hair cross sections before and after UVA and UVB irradiation. Hair was coated using either basic oxidative polymerization of DA or chemoenzymatic oxidative polymerization of DA and TA. All settings, including magnification were kept constant and at least 4 images per irradiated sample were acquired. More images are reported in the manuscript. Scale bars: 50 μm.

FIGS. 44A-44D. Optical micrographs of hair dyed via enzymatic oxidation of (FIG. 44A) L-Tyr, (FIG. 44B) TA, (FIG. 44C) DA before and after 5 washes with water and 10 washes with 10% shampoo solution. FIG. 44D. Example of 10% shampoo solution before and hair wash.

FIG. 45. Example of optical reflectance measurement. The three highlighted spots indicate the positions of each acquired optical reflectance measurement. Results were reported as an average of the three measurements.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The term “melanin” generally refers to one or more compounds or materials that function as a pigment, such as when internalized or taken up by a biological cell, for example. It is also noted that melanin is not necessarily taken up by cells. Melanin can be used for forming cell walls in fungi, for example, such as to provide rigidity, defense mechanisms, and more. In another illustrative example, melanin is used by birds, such as where melanin is organized in a matrix of keratin or similar type of biological material, where it can be organized into monolayers or multilayers to provide structural color, warmth, and more. A melanin compound or material may be, but is not limited to, a melanin monomer, a melanin oligomer, a melanin polymer, a melanin nanoparticle, a melanin layer (e.g., a melanin thin film or coating), or other melanin material, for example. For example, melanin nanoparticles internalized by a biological cell function as a pigment in the cell.

The terms “artificial melanin” and “synthetic melanin” are used interchangeably herein and refer to one or more melanin compounds, molecules, or materials, such as melanin monomers, melanin oligomers, or melanin nanoparticles, that are synthesized and are at least partially, or preferably entirely, not derived from or not extracted from a natural source, such as a biological source, a living organism, or a once living organism. The terms “synthetic” and “artificial” are used interchangeably herein when referring to a melanin or a material comprising a melanin. The terms “synthetic melanin nanoparticles” and “artificial melanin nanoparticles” are used interchangeably herein, and are intended to have the same meaning throughout the present disclosure, and refer to nanoparticles formed of artificial melanin, such as artificial melanin monomers and/or artificial melanin oligomers. The terms “synthetic melanin thin film” and “artificial melanin thin film” are used interchangeably herein, and are intended to have the same meaning throughout the present disclosure, and refer to a thin film formed of artificial melanin, such as artificial melanin monomers and/or artificial melanin oligomers. The terms “synthetic melanin layer” and “artificial melanin layer” are used interchangeably herein, and are intended to have the same meaning throughout the present disclosure, and refer to a layer formed of artificial melanin, such as artificial melanin monomers and/or artificial melanin oligomers. An artificial melanin nanoparticle, artificial melanin thin film, artificial melanin layer, and any compound, material, or formulation comprising any of these, comprises artificial melanin monomers, artificial melanin oligomers, and/or artificial melanin polymers. Optionally, an artificial melanin nanoparticle, artificial melanin thin film, artificial melanin layer, and any compound, material, or formulation comprising any of these, consists of or consists essentially of artificial melanin, such as artificial melanin monomers, artificial melanin oligomers, and/or artificial melanin polymers. Optionally, an artificial melanin nanoparticle, artificial melanin thin film, artificial melanin layer, and any compound, material, or formulation comprising any of these, is free (or substantially free) of artificial melanin monomers and comprises artificial melanin oligomers and/or artificial melanin polymers. Preferably, each artificial melanin monomer, artificial melanin oligomer, and artificial melanin polymer of an artificial melanin nanoparticle, artificial melanin thin film, artificial melanin layer, and any compound, material, or formulation comprising any of these, is not bound to, conjugated to, attached to, coated by, encompassed by or chemically otherwise associated with a natural or biological proteinaceous lipid. A natural or biological proteinaceous lipid refers to a naturally or biologically derived lipid or a lipid extracted from a natural or biological source, such as a once living organism, said lipid comprising one or more proteins such as the lipid (plasma) membrane of a melanocyte or melanosome). Optionally, each artificial melanin monomer, artificial melanin oligomer, and artificial melanin polymer of an artificial melanin nanoparticle, artificial melanin thin film, artificial melanin layer, and any compound, material, or formulation comprising any of these, is not bound to, conjugated to, attached to, coated by, encompassed by or otherwise chemically associated with a natural or biological lipid (e.g. a lipid bilayer, lipid membrane or phospholipid compound). A natural or biological lipid refers to a naturally or biologically derived lipid or a lipid extracted from a natural or biological source, such as a once living organism. Optionally, any artificial melanin monomer, artificial melanin oligomer, and artificial melanin polymer of an artificial melanin nanoparticle, artificial melanin thin film, artificial melanin layer, and any compound, material, or formulation comprising any of these, is bound to, conjugated to, attached to, coated by, encompassed by, and/or otherwise associated with a synthetic or artificial lipid or with a synthetic or artificial phospholipid. A synthetic or artificial lipid refers to a synthesized lipid that is not derived from or is not extracted from a natural or biological source, such as a once living organism.

The term “artificial melanin precursor” refers to a compound or material that can form an artificial melanin material after a chemical reaction, such as after a chemical reaction with an oxidation agent. An artificial melanin precursor can be, but is not necessarily, itself a melanin. For example, an artificial melanin precursor can be, but is not necessarily, a melanin monomer. For example, contacting artificial melanin precursors such as melanin monomers with an oxidizing agent can result in oxidative oligomerization (or, polymerization) among the artificial melanin precursors thereby forming artificial melanin material(s).

The term “selenomelanin” refers to melanin comprising selenium. For example, a selenomelanin material comprises selenium. Preferably, a chemical formula of a selenomelanin material comprises selenium (e.g., at least one selenium atom).

In certain embodiments, the term “pheomelanin” refers to a melanin whose chemical formula comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these. In certain embodiments, the term pheomelanin refers to a melanin made from L-DOPA and cysteine, whose chemical formula comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these. In certain embodiments, a selenium pheomelanin refers to a melanin whose chemical formula comprises at least one substituted or unsubstituted benzoselenazole, at least one substituted or unsubstituted benzoselenazine, at least one derivative of any of these, or any combination of these.

In certain embodiments, the term eumelanin refers to a melanin whose chemical formula comprises at least one dihydroxyindole (DHI) (e.g., 5,6-dihydroxyindole), at least one dihydroxyindole-2-carboxylic acid (DHICA) (e.g., 5,6-dihydroxyindole-2-carboxylic acid), or a combination of these.

As used herein, treatment of hair and treating hair refer to changing a color of hair, such as, but not necessarily, making hair darker, such as, but not necessarily, more brown or more black, and/or increasing persistence of a hair color, such as, but not necessarily, increasing the persistence of a dark color (e.g., brown or black) of hair that is initially the dark color. Any of the methods of treating hair and any of the artificial melanin materials disclosed herein can be used for treating hair. A change in a hair color as a result of treating hair preferably, but not necessarily, corresponds to a darkening of the hair color. A change in a hair color as a result of treating hair preferably, but not necessarily, corresponds to the hair color becoming more brown or more dark. Preferably, but not necessarily, a change in a hair color as a result of treating hair corresponds to any change in color of the hair. Exemplary changes in hair color as a result of treating hair, such as according to embodiments disclosed herein, include, but are not limited to, hair becoming: a dark brown color, a brown color with shades of red, an orange or dark orange color, a brown color, a brown color with orange shades, and/or a bright blond color.

The term “persistence” of a hair colors refers to limited, low, or lack of change in the hair color, such as in response to time and/or exposure to one or more conditions or processes that can otherwise affect hair color, such as rinsing and/or washing of the hair with a solvent (e.g., water) and/or a surfactant (e.g., shampoo). A hair color or a change in hair color characterized as persistent refers to the hair color or the change in hair color having persistence. A persistent change in hair color refers to the hair color resulting from the treating of the hair (e.g., the color obtained after treating hair) having persistence. Hair color persistence can be characterized by absolute and/or relative change, if any, of RGB color intensities and/or RGB color ratios corresponding to the hair color.

The term “aging”, when used in reference to artificial melanin nanoparticles herein, refers to a process by which synthesized and isolated artificial melanin nanoparticles oxidize, and optionally further darker, over time during exposure to oxygen, such due to exposure to air. Isolated artificial melanin nanoparticles can be artificial melanin nanoparticles that are purified, such as by centrifugation, and re-dispersed in water, such as ultrapure water, or optionally another solvent or solvent solution. For example, artificial melanin nanoparticles may age if the particles are dispersed in water and are stored in a vial with the vial's top on (closed) and with the top not being opened for some extended period of time, because there is residual oxygen in the container. The aging process can alter certain properties or characteristics of artificial melanin nanoparticles, such as increasing solubility in organic solvent or decreasing toxicity to certain living biological cells. For example, without wishing to be bound by any particular theory, in some embodiments, freshly synthesized artificial melanin nanoparticles can be dynamic and shed monomers or oligomers into a cell when internalized by the cell. For example, without wishing to be bound by any particular theory, in some embodiments, freshly synthesized artificial melanin nanoparticles can be dynamic and have surface chemistry oxidation state that is not optimal for living cells when internalized by cells. For example, without wishing to be bound by any particular theory, in some embodiments, the aging process can lead to more crosslinking or otherwise chemical association between melanin compounds (monomers, oligomers) in the artificial melanin nanoparticles, potentially leading to reduced cytotoxicity, such as due to reduced shedding of melanin compounds into the cell and/or altering or stabilizing of the particles' surface chemistry.

The term “nanoparticle” as used herein, refers to a physical particle having at least one size characteristic or physical dimension less than less than 1 μm. Preferably, term “nanoparticle” as used herein, refers to a physical particle whose longest size characteristic or physical dimension is less than 1 μm.

The term “size characteristic” refers to a property, or set of properties, of a particle that directly or indirectly relates to a size attribute. According to some embodiments, a size characteristic corresponds to an empirically-derived size characteristic of a particle(s) being detected, such as a size characteristic based on, determined by, or corresponding to data from any technique or instrument that may be used to determine a particle size, such as electron microscope (e.g., SEM and TEM) or a light scattering technique (e.g., DLS). For example, a size characteristic can correspond to a spherical particle exhibiting similar or substantially same properties, such as aerodynamic, hydrodynamic, optical, and/or electrical properties, as the particle(s) being detected). According to some embodiments, a size characteristic corresponds to a physical dimension, such as a cross-sectional size (e.g., length, width, thickness, or diameter).

The term “particles” refers to small solid objects that may be dispersed and/or suspended in a fluid (e.g., liquid). For example, a slurry, a dispersion, and a suspension each include particles in a fluid. The terms “particle” and “particulate” may be used interchangeably. An exemplary particle is an artificial melanin nanoparticle. A plurality of particles may be associated together to form an agglomerate of particles. Generally, the term “particle”, such as “nanoparticle” or “melanin nanoparticle”, refers to an individual particle rather than to an agglomerate of such individual particles.

The term “dispersed” refers to species, such as particles, in a fluid forming a dispersion. As used herein, the term “dispersion” broadly refers to a mixture of one or more chemical species, such as particles, in a fluid, such as the art-recognized meaning of solution, dispersion, and/or suspension. The chemical species, such as particles, dispersed in a dispersion can be referred as a dispersed species. Preferably, a dispersion is a mixture of particles, such as artificial melanin particles, in a liquid, such as a solvent. Preferably, but not necessarily, a dispersion is a homogeneous mixture. In the context of a dispersion, the term “homogeneous” refers to a liquid mixture that appears uniform to the naked eye. In contrast, a heterogenous liquid mixture includes particles that are precipitated from or suspended in the liquid mixture and are large enough to be distinctly identifiable by the naked eye in the liquid mixture. A heterogeneous liquid mixture includes, for example, sedimented and/or sedimenting particles. Preferably, but not necessarily, the term “dispersion” is broadly intended to include solutions and dispersions, such as colloids, which are not heterogenous liquid mixtures. Preferably, but not necessarily, a dispersion is a microscopically homogenous, or uniform, mixture of particles in a liquid, such as a solvent. Preferably, but not necessarily, a dispersion is thermodynamically favored remain stably dispersed or is thermodynamically favored to segregate by sedimentation but wherein sedimentation is kinetically slowed or prevented. Particles, of a dispersion, that are characterized as stably dispersed remain dispersed in the dispersion and do not sediment or precipitate out of the liquid, of the dispersion, for at least 5 hours, preferably at least 12 hours, preferably at least 24 hours, and more preferably at least 1 week, under normal temperature and pressure (NTP) and exposure to air. In embodiments, particles that are not or cannot be dispersed in a fluid refer to particles that form precipitates or sediments upon being mixed in the fluid.

The term “size stable” refers to stability of particles in a dispersion with respect to a size characteristic of said particles. Preferably, particles in a dispersion characterized as size stable are characterized by a size characteristic being within 50%, within 40%, within 30%, preferably within 20%, more preferably within 15%, still more preferably within 10%, further more preferably within 5%, or equivalent to a reference or initial size characteristic, under given conditions and optionally for a given time. For example, nanoparticles of a dispersion characterized as size-stable in the dispersion having a pH of at least 11, with respect to an average size of the nanoparticle in the dispersion having a pH of 7, have an average size in the pH 11 dispersion that is within 50%, within 40%, within 30%, preferably within 20%, more preferably within 15%, still more preferably within 10%, further more preferably within 5%, or equivalent to an average size of the otherwise equivalent nanoparticles in the otherwise equivalent dispersion having a pH of 7. Preferably, but not necessarily, nanoparticles characterized as size stable as so size stable for time that is at least 1 hour to 5 hours, preferably at least 5 hours to 12 hours, more preferably at least 12 hours to 1 week, still more preferably at least 1 week.

The term “strong oxidizing agent” refers to a substance (e.g., compound, molecule, material) having a greater ability for subtracting, removing, or accepting one or more electron from another other substance compared to oxygen gas, including oxygen gas dissolved in a solution. The greater ability may be due to thermodynamic, kinetic, and/or electrochemical characteristics thereof. Optionally, a strong oxidizing agent has a greater or more positive standard electrode potential than O₂.

The term “U” in a unit of concentration, such as “U/mL”, refers to “unit of activity” and is a known term of art referring to enzyme catalytic activity. A unit “U” refers to the amount of enzyme that catalyzes the conversion of 1 micromole (μmole) of a substrate per minute. Thus, 1 enzyme unit (U)=1 μmol/min, where μmol refers to the amount of substrate converted. Because each enzyme has a unique substrate, a unit of activity is different for one enzyme versus another.

The term “structural color” refers to the generation of color due to interference of visible light structural features, such as a film or layer or a microstructured surface. A layer of melanin nanoparticles may exhibit color due to interference of visible light with the microstructure of the layer, rather than solely due to pigmentation. Without wishing to be bound by any particular theory, the effect of structural color can enable a spectrum on non-fading, non-photobleaching colors which can be iridescent or non-iridescent. Without wishing to be bound by any particular theory, high refractive index of melanin and synthetic melanin, and its broadband absorption across the visible spectrum allows it to interact with light in such a way that a multitude of colors are produced.

The term “peak size” size refers to the statistical mode, or peak frequency, of a particle size distribution, or the particle size most commonly found in the particle size distribution. A particle size distribution can be measured using dynamic light scattering, for example.

The term “sphere” as used herein, in the usual and customary sense, refers to a round or substantially round geometrical object in three-dimensional space that is substantially the surface of a completely round ball, analogous to a circular object in two dimensions. A sphere may be defined mathematically as the set of points that are all at the same or substantially all at the same distance r from a given point, but in three-dimensional space, where r is the radius of the mathematical ball and the given point is the center or substantially the center of the mathematical ball. In embodiments, the longest straight line through the ball, connecting two points of the sphere, passes through the center and its length is thus twice the radius; it is a diameter of the ball. A nanosphere is a nanoparticle having a radius of less than 1 μm.

The terms “ultraviolet induced damage” and “UV induced damage” as used interchangeably herein refer, in the usual and customary sense, to chemical changes attending irradiation of light of sufficient energy. UV induced damage can include scission of nucleic acids (e.g., DNA or RNA), and breaking of bonds in proteins, lipids, and other physiological molecules. For example, the damage can be damage resulting from reactive oxygen species (ROS).

The terms “reactive oxygen species” and “ROS” as used interchangeably herein refer, in the usual and customary sense, to transient species, typically formed during exposure to radiation (e.g., UV irradiation) capable of inducing oxidative decomposition.

The terms “cell” and “biological cell” are used interchangeably are refer to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. A “viable cell” is a living biological cell.

The term “self-assembly” refers to a process in which individual elements assemble into a network or organized structure without external direction. In an embodiment, self-assembly leads to a decrease in entropy of a system. In an embodiment, self-assembly may be induced, or initiated, via contacting or reacting the individual elements, optionally at a certain critical concentration, and/or via temperature and/or via pressure. A “self-assembled structure” is a structure or network formed by self-assembly. In an embodiment, self-assembly is a polymer crystallization process. The Gibbs free energy of the self-assembled structure is lower than of the sum of the individual components in their non-organized arrangement prior to self-assembly under otherwise identical conditions (e.g., temperature and pressure). In an embodiment, entropy of a self-assembled structure is lower than that of the sum of the individual components in their non-organized arrangement prior to self-assembly under otherwise identical conditions (e.g., temperature and pressure). For example, artificial melanin nanoparticles of this disclosure can form by self-assembly of a plurality of oligomers and/or melanin monomers. For example, structures or layers (e.g., films) for artificial melanin nanoparticles may form by self-assembly, such as structures or layers formed of artificial melanin nanoparticles and exhibiting structural color.

The term “substantially” refers to a property, condition, or value that is within 20%, 10%, within 5%, within 1%, optionally within 0.1%, or is equivalent to a reference property, condition, or value. The term “substantially equal”, “substantially equivalent”, or “substantially unchanged”, when used in conjunction with a reference value describing a property or condition, refers to a value that is within 20%, within 10%, optionally within 5%, optionally within 1%, optionally within 0.1%, or optionally is equivalent to the provided reference value. For example, a diameter is substantially equal to 100 nm (or, “is substantially 100 nm”) if the value of the diameter is within 20%, optionally within 10%, optionally within 5%, optionally within 1%, within 0.1%, or optionally equal to 100 nm. The term “substantially greater”, when used in conjunction with a reference value describing a property or condition, refers to a value that is at least 1%, optionally at least 5%, optionally at least 10%, or optionally at least 20% greater than the provided reference value. The term “substantially less”, when used in conjunction with a reference value describing a property or condition, refers to a value that is at least 1%, optionally at least 5%, optionally at least 10%, or optionally at least 20% less than the provided reference value.

The terms “keratinocyte” and “keratinocytes” as used herein, refer to the predominant cell type in the epidermis, the outermost layer of the skin, constituting the majority (e.g., 90%-95%) of the cells found there. Keratinocytes are found in the deepest basal layer of the stratified epithelium that comprises the epidermis, and are sometimes referred to as basal cells or basal keratinocytes. Keratinocytes are maintained at various stages of differentiation in the epidermis and are responsible for forming tight junctions with the nerves of the skin. They also keep Langerhans cells of the epidermis and lymphocytes of the dermis in place. Keratinocytes contribute to protecting the body from UV radiation by taking up melanosomes. Keratinocytes contribute to protecting the body from UV radiation by taking up melanosomes, vesicles containing the endogenous photoprotectant melanin, from epidermal melanocytes. Each melanocyte in the epidermis has several dendrites that stretch out to connect it with many keratinocytes. The melanin is then stored within keratinocytes and melanocytes in the perinuclear area as “supranuclear caps”, where it protects the DNA from UV-induced damage. In addition to their structural role, keratinocytes play a role in immune system function. The skin is the first line of defense and keratinocytes serve as a barrier between an organism and its environment. In addition to preventing toxins and pathogens from entering an organisms body, they prevent the loss of moisture, heat and other important constituents of the body. In addition to their physical role, keratinocytes serve a chemical immune role as immunomodulators, responsible for secreting inhibitory cytokines in the absence of injury and stimulating inflammation and activating Langerhans cells in response to injury. Langerhans cells serve as antigen-presenting cells when there is a skin infection and are the first cells to process microbial antigens entering the body from a skin breach.

The terms “under conditions suitable to afford uptake”, “taken up” and “take up” as used herein, refer, in the usual and customary sense, to experimental conditions well known in the art which allow uptake (e.g., endocytosis) of a species into a cell. In some embodiments, the term “internalized” when referring to particles internalized in or by a biological cell, refers to particles taken up by the biological cell, such as by, but not limited to, formation of perinuclear caps.

The term “endocytosis” as used herein, refers to a form of active transport in which a cell transports molecules (such as proteins) into the cell by engulfing them in an energy-using process. Endocytosis includes pinocytosis and phagocytosis. Pinocytosis is a mode of endocytosis in which small particles are brought into the cell, forming an invagination, and then suspended within small vesicles. These pinocytotic vesicles subsequently fuse with lysosomes to hydrolyze (break down) the particles. Phagocytosis is the process by which a cell engulfs a solid particle to form an internal compartment known as a phagosome.

The terms “treating” or “treatment” as used herein, refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating,” and conjugations thereof, include prevention of an injury, pathology, condition, or disease.

The term “effective amount” as used herein, refers to an amount sufficient to accomplish a stated purpose (e.g. Achieve the effect for which it is administered, treat a disease, reduce one or more symptoms of a disease or condition, and the like). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “administering” as used herein, refers to oral administration, administration as an inhaled aerosol or as an inhaled dry powder, suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compound of the invention can be administered alone or can be co-administered to the patient. Co-administration is meant to include simultaneous or sequential administration of the compound individually or in combination (more than one compound or agent). The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293306, 1996; Chonn, Curr. Opin. Biotechnol. 6:698-708, 1995; Qstio, Am. J Hasp. Pharm. 46: 1576-1587, 1989).

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell or a patient.

The terms “analog” and “analogue” are used interchangeably and are used in accordance with their plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof. Accordingly, an analog is a compound that is similar or comparable in function and appearance but not in structure or origin to a reference compound.

Except where otherwise specified, the term “molecular weight” refers to an average molecular weight. Except where otherwise specified, the term “average molecular weight,” refers to number-average molecular weight. Number average molecular weight is defined as the total weight of a sample volume divided by the number of molecules within the sample. As is customary and well known in the art, peak average molecular weight and weight average molecular weight may also be used to characterize the molecular weight of the distribution of polymers within a sample.

The term “weight-average molecular weight” (M_{w}) refers to the average molecular weight defined as the sum of the products of the molecular weight of each polymer molecule (Mᵢ) multiplied by its weight fraction (wᵢ): M_{w}=ΣwᵢMᵢ. As is customary and well known in the art, peak average molecular weight and number average molecular weight may also be used to characterize the molecular weight of the distribution of polymers within a sample.

The term “wt. %” or “wt %” refers to a weight percent, or a mass fraction represented as a percentage by mass. The term “at. %” or “at %” refers to an atomic percent, or an atomic ratio represented as a percentage of a type of atom with respect to total atoms in a given matter, such as a molecule, compound, material, nanoparticle, polymer, dispersion, etc.

The term “oligomerization” refers to a chemical process of converting a monomer or a mixture of monomers into an oligomer. The term “oxidative oligomerization” refers to a chemical process of oligomerization that includes chemical oxidation of one or more monomers to form an oligomer. An oligomerization is a polymerization process, wherein an oligomer is formed as a result of the polymerization.

As used herein, the term “polymer” refers to a molecule composed of repeating structural units connected by covalent chemical bonds often characterized by a number of repeating units, also referred to as base units (e.g., greater than or equal to 2 base units). As used herein, a term “polymer” is inclusive of an “oligomer” (i.e., an oligomer is a polymer; i.e., a polymer is optionally an oligomer). An “oligomer” refers to a molecule composed of repeating structural units, also referred to as base units, connected by covalent chemical bonds often characterized by a number of repeating units less such that the oligomer is a low molecular weight polymer. Preferably, but not necessarily, for example, an oligomer has equal to or less than 100 repeating units. Preferably, but not necessarily, for example, an oligomer has a lower molecular weight less than or equal to 10,000 Da. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units. Polymers can have, for example, greater than 100 repeating units. Polymers can have, for example, a high molecular weight, such as greater than 10,000 Da, in some embodiments greater than or equal to 50,000 Da or greater than or equal to 100,000 Da. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits, and include random, block, brush, brush block, alternating, segmented, grafted, tapered and other architectures. Useful polymers include organic polymers or inorganic polymers that may be in amorphous, semi-amorphous, crystalline or semi-crystalline states. Polymer side chains capable of cross linking polymers (e.g., physical cross linking) may be useful for some applications.

An “oligomer” refers to a molecule composed of repeating structural units, also referred to as base units, connected by covalent chemical bonds often characterized by a number of repeating units less than that of a polymer (e.g., equal to or less than 100 repeating units) and a lower molecular weights (e.g. less than or equal to 10,000 Da) than polymers. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

The term “moiety” refers to a group, such as a functional group, of a chemical compound or molecule. A moiety is a collection of atoms that are part of the chemical compound or molecule. The present invention includes moieties characterized as monovalent, divalent, trivalent, etc. valence states. Generally, but not necessarily, a moiety comprises more than one functional group.

As used herein, the term “substituted” refers to a compound wherein one or more hydrogens is replaced by another functional group, provided that the designated atom's normal valence is not exceeded. An exemplary substituent includes, but is not limited to: a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. In some embodiments, the term substituted refers to a compound wherein each of more than one hydrogen is replaced by another functional group, such as a halogen group. For example, when the substituent is oxo (i.e., ═O), then two hydrogens on the atom are replaced. The substituent group can be any substituent group described herein. For example, substituent groups can include one or more of a hydroxyl, an amino (e.g., primary, secondary, or tertiary), an aldehyde, a carboxylic acid, an ester, an amide, a ketone, nitro, an urea, a guanidine, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

As used herein, the term “derivative” refers to a compound wherein an atom or functional group is replaced by another atom or functional group (e.g., a substituent function group as also described below), including, but not limited to: a hydrogen, a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. Preferably, the term “derivative” refers to a compound wherein one or two atoms or functional groups are independently replaced by another atom or functional group. Optionally, the term derivative does not refer to or include replacement of a chalcogen atom (S, Se) that is a member of a heterocyclic group. Optionally, and unless otherwise stated, the term derivative does not refer to or include replacement of a chalcogen atom (S, Se) nor a N (nitrogen) where the chalcogen atom and the N are members same heterocyclic group. Optionally, but not necessarily, the term derivative does not include breaking a ring structure, replacement of a ring member, or removal of a ring member.

As is customary and well known in the art, hydrogen atoms in formula, are not always explicitly shown, for example, hydrogen atoms bonded to the carbon atoms of aromatic, heteroaromatic, and alicyclic rings are not always explicitly shown. The structures provided herein, for example in the context of the description of formula and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups.

Compounds of the invention may have substituted and/or unsubstituted C₁-C₂₀ alkylene, alkylene and C₁-C₅ alkylene groups, for example, as one or more linking groups (e.g. L¹-L⁶).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g. L¹-L⁶).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g. L¹-L⁶).

As used herein, the terms “heteroarylene” and “heteroarylene group” are used synonymously and refer to a divalent group derived from a heteroaryl group as defined herein. The invention includes compounds having one or more heteroarylene groups. In an embodiment, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, heteroarylene and C₃-C₅ heteroarylene groups, for example, as one or more linking groups (e.g. L¹-L⁶).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g. L¹-L⁶).

As used herein, the terms “cylcoalkenylene” and “cylcoalkenylene group” are used synonymously and refer to a divalent group derived from a cylcoalkenyl group as defined herein. The invention includes compounds having one or more cylcoalkenylene groups. Cycloalkenylene groups in some compounds function as linking and/or spacer groups. Compounds of the invention include substituted and/or unsubstituted C₃-C₂₀ cylcoalkenylene, C₃-C₁₀ cylcoalkenylene and C₃-C₅ cylcoalkenylene groups, for example, as one or more linking groups (e.g. L¹-L⁶).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g. L¹-L⁶).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, threonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides and peptide moieties, as used and described herein, comprise two or more amino acid groups connected via peptide bonds.

Amino acids and amino acid groups refer to naturally-occurring amino acids, unnatural (non-naturally occurring) amino acids, and/or combinations of these. Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gin), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to herein by either the commonly known three letter symbols or by the one-letter symbols recommended by the IUPAC-IUB Biochemical Nomenclature Commission.

The terms “monomer unit,” “repeating monomer unit,” “repeating unit,” and “polymerized monomer” can be used interchangeably and refer to a monomeric portion of a polymer described herein which is derived from or is a product of polymerization of one individual “monomer” or “polymerizable monomer.” Each individual monomer unit of a polymer is derived from or is a product of polymerization of one polymerizable monomer. Each individual “monomer unit” or “repeating unit” of a polymer comprises one (polymerized) polymer backbone group. For example, in a polymer that comprises monomer units X and Y arranged as X-Y-X-Y-X-Y-X-Y (where each X is identical to each other X and each Y is identical to each other Y), each X and each Y is independently can be referred to as a repeating unit or monomer unit.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, 7-, or 8-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO—refers to CH₃O—. Compositions of some embodiments of the invention comprise alkyl groups as terminating groups, such as polymer backbone terminating groups and/or polymer side chain terminating groups. Substituted alkyl groups may include substitution to incorporate one or more silyl groups, for example wherein one or more carbons are replaced by Si.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6-7-, or 8-member aromatic rings, including heterocyclic aromatic rings. The term heteroaryl specifically refers to aryl groups having at least one 5-, 6-7-, or 8-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those that are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-member ring and one or more additional five- or six-member aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. In addition, the compounds of this invention include all stereochemical isomers arising from the substitution of these compounds. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others:

halogen, including fluorine, chlorine, bromine or iodine;

pseudohalides, including —CN;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms; and

—OR where R is H, an alkyl group, an aryl group, or an acyl group all of which are optionally substituted. In a particular example R can be an acyl yielding—OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

Many of the molecules disclosed herein contain one or more ionizable groups. Ionizable groups include groups from which a proton can be removed (e.g., —COOH) or added (e.g., amines) and groups that can be quaternized (e.g., amines). All possible ionic forms of such molecules and salts thereof are intended to be included individually in the disclosure herein. With regard to salts of the compounds herein, one of ordinary skill in the art can select from among a wide variety of available counterions that are appropriate for preparation of salts of this invention for a given application. In specific applications, the selection of a given anion or cation for preparation of a salt can result in increased or decreased solubility of that salt.

The compounds of this invention can contain one or more chiral centers. Accordingly, this invention is intended to include racemic mixtures, diastereomers, enantiomers, tautomers and mixtures enriched in one or more stereoisomer. The scope of the invention as described and claimed encompasses the racemic forms of the compounds as well as the individual enantiomers and non-racemic mixtures thereof.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety, functional group, atom, ion, unpaired electron, or other chemical species to the represented molecule, compound, or chemical formula. For example, in the formula

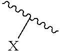

“X” represents a molecule or compound, the symbol “ [symbol] ” denotes a point of attachment of a chemical moiety, functional group, atom, ion, unpaired electron, or other chemical species to X (where X corresponds to the represented molecule, compound, or chemical formula) via covalent bonding. As used herein, the various functional groups represented will be understood to have a point of attachment at the functional group having the hyphen or dash (-) or a dash used in combination with an asterisk (*). In other words, in the case of —CH₂CH₂CH₃ or —CH₂CH₂CH₃, it will be understood that the point of attachment is the CH₂ group at the far left. If a group is recited without an asterisk or a dash, then the attachment point is indicated by the plain and ordinary meaning of the recited group.

Where substituent groups are specified by their conventional chemical formulae, written from left to right, they equally encompass the chemically identical substituents that would result from writing the structure from right to left, e.g., —CH₂O— is equivalent to —OCH₂—.

Additional embodiments and descriptions may be found in U.S. Provisional Patent Application 62/928,129, filed Oct. 30, 2019, U.S. Provisional Patent Application No. 62/868,369, filed Jun. 28, 2019, Huang, et al. (Huang, Y.; Li, Y.; Hu, Z.; Yue, X.; Proetto, M. T.; Jones, Y.; Gianneschi, N. C., Mimicking Melanosomes: Polydopamine Nanoparticles as Artificial Microparasols. ACS Cent Sci 2017, 3 (6), 564-569), and US Patent Publication No. 2020/0113934A1, all of which are incorporated herein in their entirety to the extent not inconsistent herewith.

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

Commercially available hair dyes pigments are applied in alkaline pH (ammonia or ethanolamine). This allows the permeation into the hair cortex. These compounds consists of a primary intermediate, for instance, para-phenylenediamine and para-aminophenol applied in the presence of hydrogen peroxide. After permeation into the cortex, reaction with coupling agents (resorcinol, m-aminophenol . . . ) produce the desired hair color. Due to the toxicity of some of the used components research has focused on the design of novel methods. Recent works involved the use of graphene as well as gold nanoparticles.

Melanin is the natural pigment present in the hair. Its concentration and type gives rise to different colors and its degradation results in white hair. For these reasons, developing a method to reintroduce melanin into the hair would provide a cheap and the less invasive method to color human hair. There have been attempts reporting successful polydopamine deposition on human hair. However, these methods are based on the use of metal chelation such as iron and copper to deposit the polymer on hair. Provided herein are metal-free treatment methods and materials for incorporation melanin in human hair. The metal free-deposition of synthetic melanin into human hair can be achieved by mean of three different strategies. (I) Direct deposition of polydopamine as well as derivatives in human hair, (II) covalent immobilization of the monomers on human hair by mean of thiol chemistry and consequent polymerization of polydopamine or derivatives and (III) Direct binding of pre-formed melanin (polydopamine as well as derivatives) nanoparticles to human hair.

This approach may be used for hair as well as eyebrow coloring. Protein-based surfaces (for instance silk and wool) can be colored using the deposition method developed here, either by non-covalent as well as covalent deposition.

The incorporation of synthetic melanin and derivatives offer the possibility to color white hair and clear hair with different colors (for instance blond, orange, brown, black). This method allows hair coloring within 2 h without the use of metals. The amount of base can be lowered as compared to normally employed hair dye conditions. These colors can have extend resistance to the sun light and also protect the hair from UV damage, for example, by exhibiting antioxidant properties.

This technology is based on the deposition of synthetic melanin and derivatives on human hair avoiding the use of heavy metals or reducing their use to concentration approved in cosmetic. This technology can be divided into two approaches: non-covalent and covalent deposition. The non-covalent deposition involves the in situ polymerization of dopamine or other catechol-based molecules and derivatives in human hairs. The second approach involves the covalent linkage to the human hair. This can be achieved using functionalized monomers as well as pre-formed functionalized melanin nanoparticles.

The non-covalent deposition in human hair involves the in situ polymerization of dopamine or other catechol-based molecules and derivatives using different alkaline conditions and in a range of temperatures. Alkaline conditions are generated using ammonia solutions used for conventional hair dye processes. The deposition of polydopamine on human hair within 2 hr has been performed successfully with the discovery of the role of the reaction temperature. While deposition at room temperature can be achieved only in the presence of metals (mM concentrations), a temperature range around 35-40 degrees allow hair coloring without the use of any metal already after 1-2 hr treatment, optionally for at least 2 hrs to provide uniform color. This protocol allows the use of more diluted base conditions, which is less damaging for the hair. The color is retained after several water and shampoo washes. This protocol also avoids the use of p-phenylenediamine which is toxic and irritant.

The second approach involves the covalent linkage to the human hair. This process allow for the metal-free immobilization of monomers to the hair. This can be achieved using monomers bearing thiol-reactive moieties (e.g., thiol, maleimide, pyridyl disulfide-based compound, alkene, alkyl halide, etc.) which bind to keratine proteins, rich in cysteines (with or without previous disulfide bond reduction). Upon immobilization, in situ polymerization can be achieved using above reported conditions. In a similar manner, pre-formed melanin nanoparticles containing thiol reactive moieties (e.g., thiol, maleimide, pyridyl disulfide-based compound, alkene, alkyl halide, etc.) can be conjugated to the human hair via thiol chemistry. This approach do not require any in situ polymerization and therefore can be obtained using milder conditions.

The invention can be further understood by the following non-limiting examples.

### Example 1: Mimicking Natural Human Hair Pigmentation with Synthetic Melanin

Human hair is naturally colored by melanin pigments, which affords myriad colors from black, to brown, to red depending on the chemical structures and specific blends. In recent decades, synthetic efforts have centered on dopamine oxidation to polydopamine, an effective eumelanin similar to the one found in humans. To date, only few attempts at polydopamine deposition on human hair have been reported, and their translation to widespread usage and potential commercialization is still hampered by the harsh conditions employed. We reasoned that novel, mild, biocompatible approaches could be developed to establish a metal-free route to tunable, nature-inspired, long-lasting coloration of human hair. Herein, we describe synthetic and formulation routes to achieving this goal and show efficacy on a variety of human hair samples via multiple spectroscopy and imaging techniques. This novel approach has the potential to replace classical harsh hair dyeing conditions that have raised concerns for several decades due to their potential toxicity.

Human hair is comprised mainly of protein, at 65-95% by weight. Keratin, the most abundant component, is a group of insoluble protein complexes which impart elasticity, suppleness and resistance to the fibers.(1) Melanin, nature's hair pigment, is mainly distributed in the middle layer of the hair shaft or cortex and is embedded between keratin fibers, where it makes up only 1 to 3% of human hair by weight. These nanometer-scale granular pigments (200-800 nm) generate the naturally beautiful colors found in human hair. Colors arise from the distribution, concentration, and blending of two types of melanin: brown and black eumelanins, and less commonly, red pheomelanins.(2-5) It follows then that the reduction or disappearance of melanin from hair fibers is the phenomenon that leads to color loss and consequent hair greying and eventually whitening.(3)

Hair whitening is mainly caused by aging, although the regulation of hair pigmentation and pigment concentration can be affected by numerous factors including metabolism, hair-cycle changes, body distribution of melanins, gender differences, and the use of medicines (e.g. chemotherapy), or by specific genetic disorders.(3, 6, 7) Taking these factors into account, the average age for white hair onset is the mid-30s, with 50% of people exhibiting 50% grey hair by the time they are 50 years of age.(3, 8) The first known example of natural hair dye dates back to the ancient Egyptians when henna plant pigments were used for hair darkening and color reinforcement.(9) Dye technologies are at the very origin of the chemical industry, with the first artificial “long lasting” hair dye synthesized by L'Oréal founder Eugène Schueller in the early 1900's.(9) Since then, hair dyes capable of providing a long lasting and convincing grey to black transition have become popular across cultures and nationalities, with additional colors, including those beyond one's genetic predisposition, also being desirable. Due to such widespread use, hair dye industries are now among the most profitable in the cosmetics sector.(10) As a matter of fact, studies suggest that over 50% of the population in developed countries has dyed their hair at least once in their life.(5, 11-14) Despite several studies reporting the potential carcinogenicity of certain conventional hair dye components, frequent development of allergies in clients and colorists, and dye-induced hair damage, the use of small molecule-based dyes in modern society continues to expand, and the industry has made only few minor advances in its chemistry.(4, 12, 14-18)

With the rapid expansion of nanotechnology, a field at the intersection of chemistry and materials science, novel creative approaches can now be exploited for the design of new hair dyes. This approach is even more interesting with the discovery that ancient hair dying methods also relied on nanostructure deposition.(19) While the synthesis of gold nanoparticles in human hair has been proposed as an effective way to darken white hair,(20) the long reaction time required by this protocol (16 days) hampers its application as an effective hair dyeing method. A much faster approach involves the use of graphene-based sheets for coloration. Hair coated with this material showed good antistatic performance and heat dissipation properties, however, the method was similarly expensive and only produced a single color, black.(21) Although both of these nanomaterial-based methods provide paths toward the development of innovative hair dyeing protocols, a very interesting approach to darken hair and a desirable alternative to current formulations would be a synthetic version of the naturally occurring nano-sized melanin pigment particles to reestablish color of the hair fibers. This approach is now feasible owing to myriad examples in the literature describing the synthesis and applications of artificial melanin.(22) Successful human hair dyeing using synthetic melanin was attempted only very recently,(23, 24) and required high concentrations of potentially toxic heavy metals such as copper and iron as chelators.(25) Moreover, these two studies were limited to a dark brown coloration and lack extensive imaging and characterization of the dyeing mechanism. Very recently, strong oxidative conditions using sodium periodate were employed for successful dopamine deposition on human hair,(26) but this method may not be suitable for widespread use in an at-home or salon application.(27)

Herein, we demonstrate the efficient deposition of synthetic melanin to human hair without the need for metal chelators or strong oxidants to generate not only black/brown, but also orange/gold colorations from blond hair, for example. We demonstrate that different colors can be achieved by tuning reaction conditions such as temperature, and that effective hair dyeing can be achieved using milder conditions compared to those previously employed for polydopamine coatings. Moreover, these conditions are similar or even milder than that employed for conventional hair dyeing protocols. In addition, blond and naturally red, brown and grey hair, as well as hair previously dyed with a very bright commercial dye were successfully colored to dark brown/black using this method. These results demonstrate that this novel, mild and versatile nature-inspired approach to hair pigmentation can replace classical, harsh hair dyes currently common in the cosmetic industry.

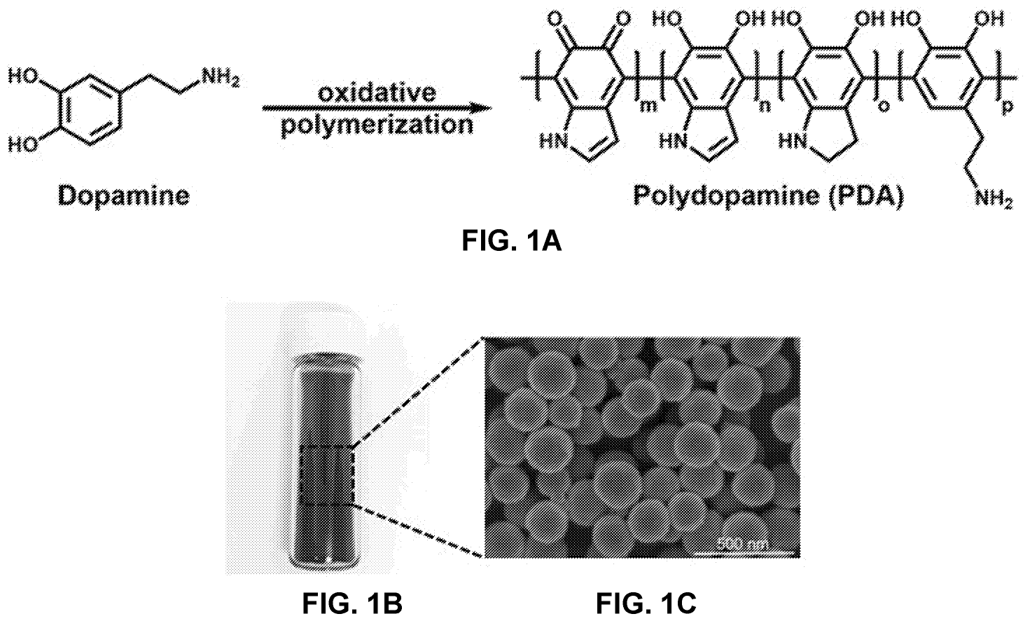

In humans, the biosynthesis of melanin, a rather heterogeneous and polydisperse polymer mainly composed of polydopamine derivatives, begins with the enzymatic oxidation of L-tyrosine to 3,4-dihydroxyphenylalanine (L-DOPA). This process occurs inside melanocytes in specialized organelles called melanosomes.(4, 28, 29) The resulting dark melanin granules are transferred to hair and the epidermis from the melanocytes, by a process in which melanosomes are endocytosed by epithelial cells.(29) The broad applications of polydopamine-based materials, which mimic the natural melanin abundant in hair and skin, have rapidly advanced in recent years. As a result, several synthetic strategies are now available for the preparation of this class of biopolymers.(22) In particular, synthetic versions mimicking eumelanin, the darkest natural pigment of this class, can be obtained via oxidation of commercially available dopamine (DA) hydrochloride as well as analogues, by various synthetic protocols (FIGS. 1A-1C).(22),(30)

Synthetic approaches using the enzymes laccase and horseradish peroxidase, strong oxidants such as ammonium persulfate, sodium periodate and potassium permanganate, and metal catalysts have been widely explored and optimized for the polymerization of dopamine.(22) However, for biocompatibility, the auto-oxidation of dopamine in air is the most interesting and gentle approach for generating polydopamine (PDA) coatings or nanoparticles. This oxidation is spontaneous when carried out under alkaline conditions (pH >7.5) using naturally ambient oxygen, making this method mild, inexpensive and scalable.(22, 31) When added to an alkaline solution, the polymerization of DA occurs immediately and is accompanied by a color change from clear and colorless, to pale brown, and finally to dark brown and black (PDA). Hence, we chose dopamine self-oxidation as the most promising approach for melanin deposition on human hair. While Tris buffer (pH 8.5) and NaOH are reagents used extensively for successful dopamine oxidation,(22, 31) ammonium hydroxide is an attractive choice as this base is commonly used in current hair dyeing protocols.(4, 32) Although some modern hair dye formulations are ammonia free, where ammonia is generally replaced by less volatile/odorous ethanolamine and its derivatives, these alternatives are still bases and can still create adverse reactions.(33) The use of bases in conventional hair dyeing protocols is generally needed to swell the hair cuticle, thereby allowing dye penetration into the hair. In this work, 3% ammonium hydroxide was selected as the initial, lowest effective concentration, in comparison with professional permanent hair dyes which can contain more than 10% in solution.(34) To note, that the ammonia percentage in the final formulations employed in this study might be slightly lower than what reported, since the concentration of the ammonia stock solution is 28-30% (w/v) and not 30% (w/v). To evaluate the performance of our method, we compared it to a previously established protocol using CuSO₄/H₂O₂ additives as a metal- and peroxide-based approach to polydopamine deposition.(24) A reaction time of 2 hours was selected as this is a feasible and acceptable time for hair dyeing in common practice. The concentration of dopamine was chosen as 5 mg/mL as it corresponds to the low end of other synthetic protocols.(24)

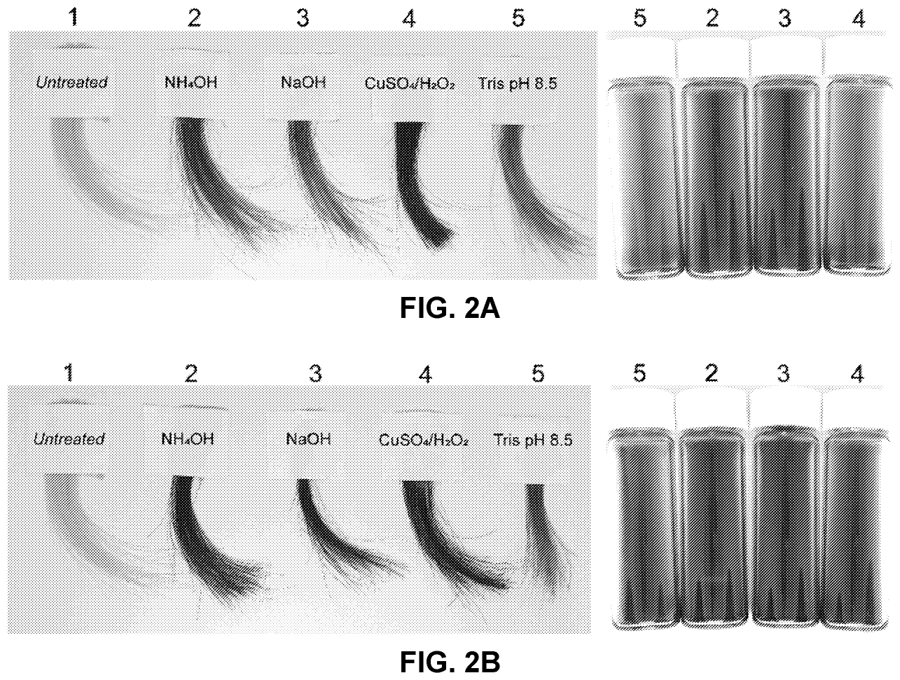

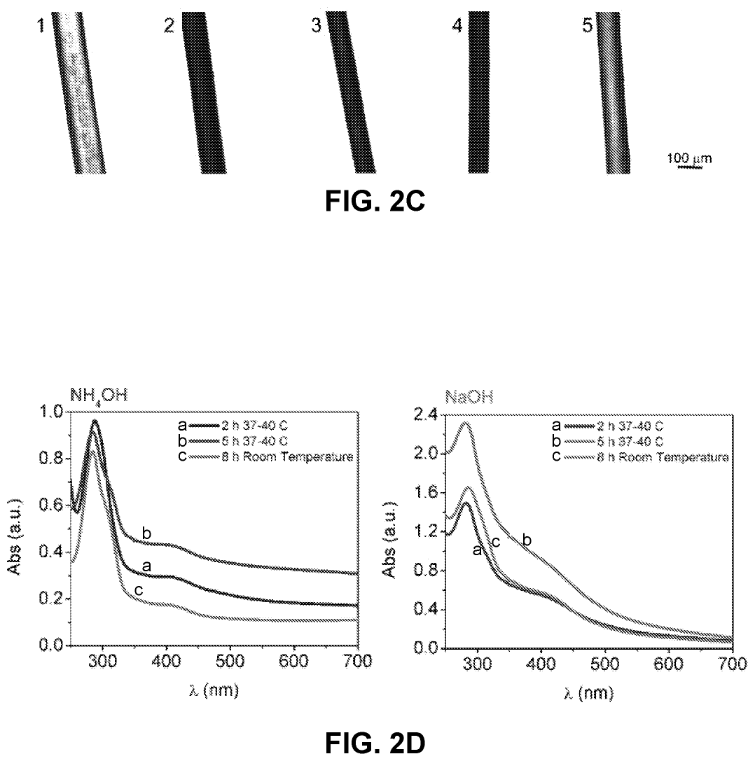

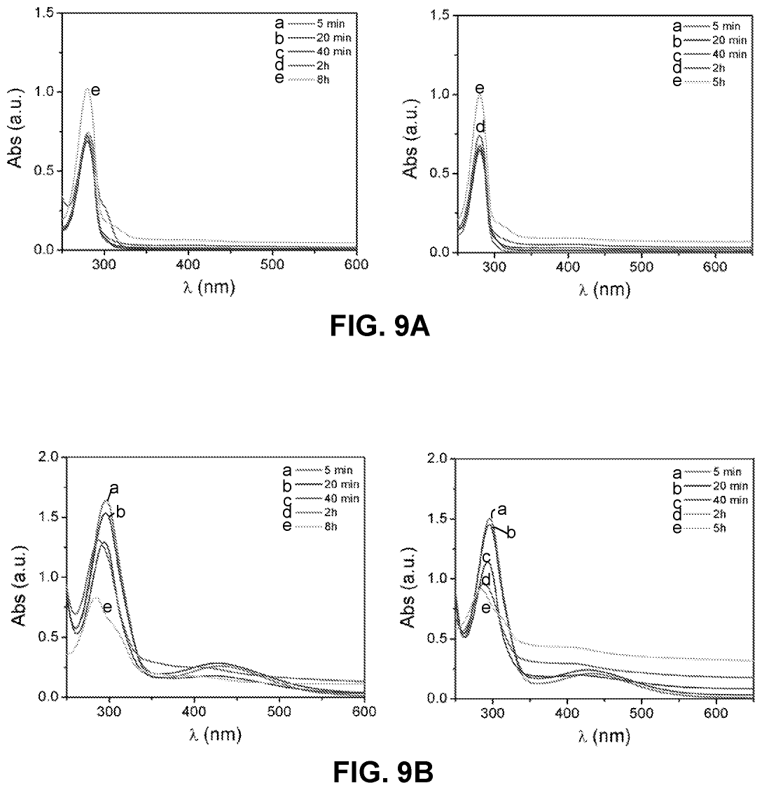

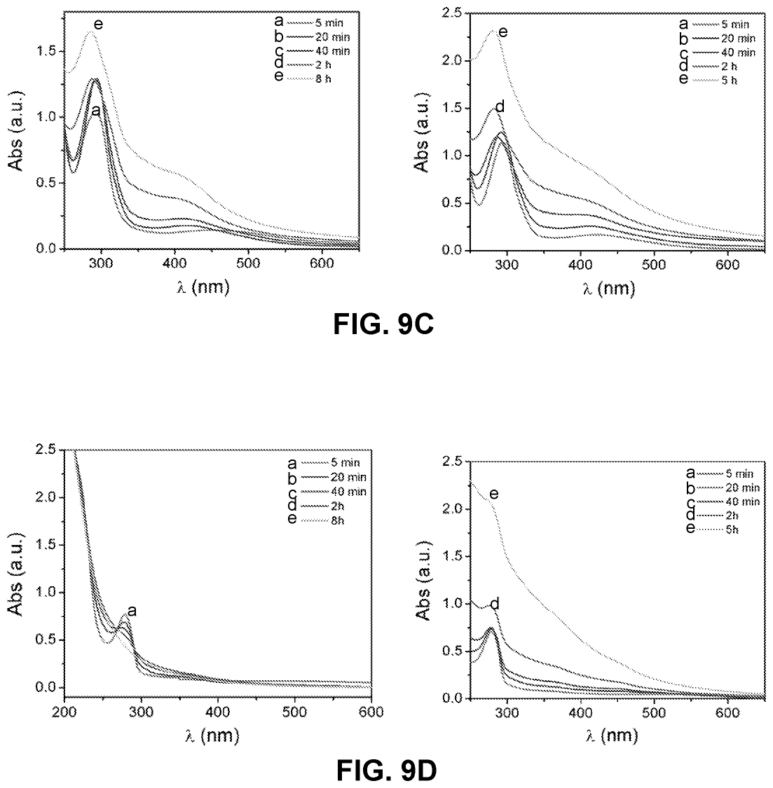

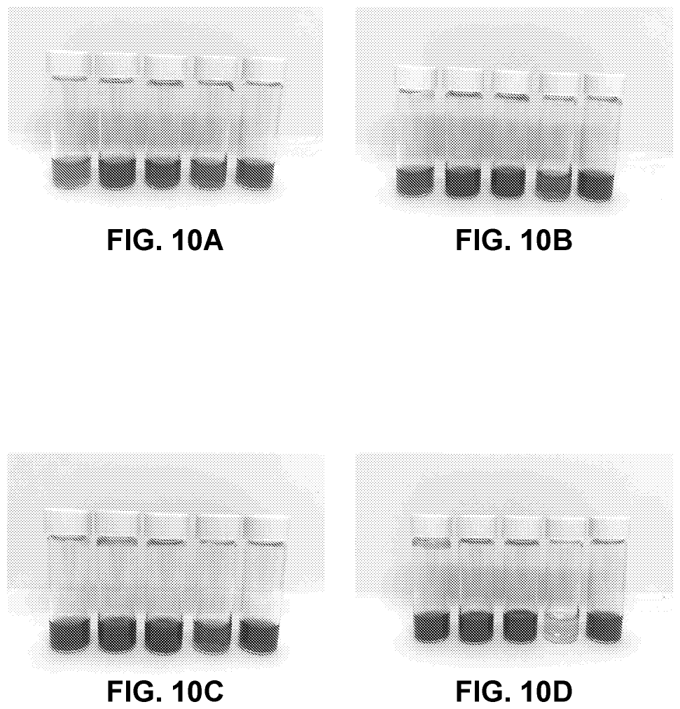

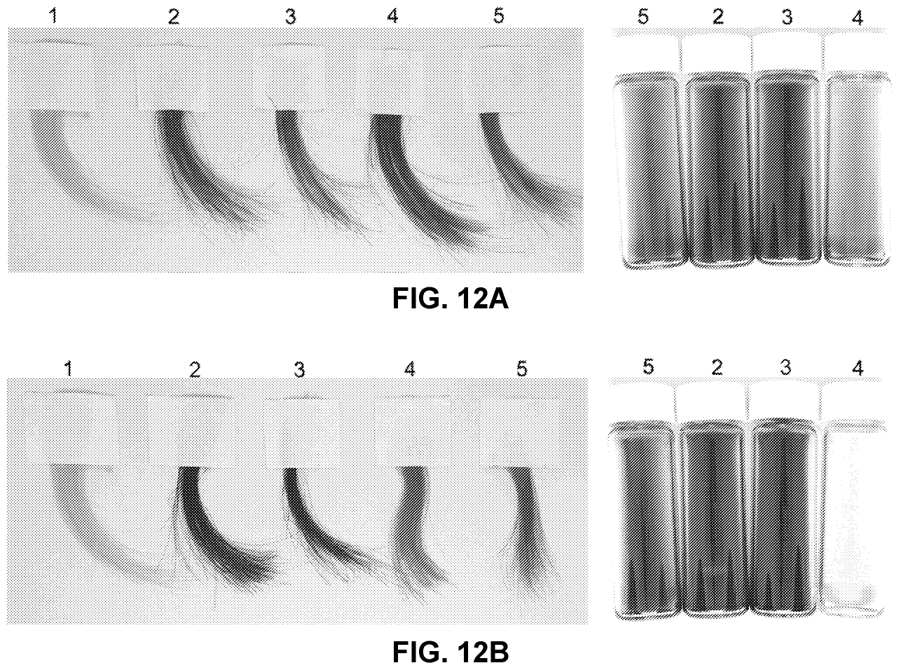

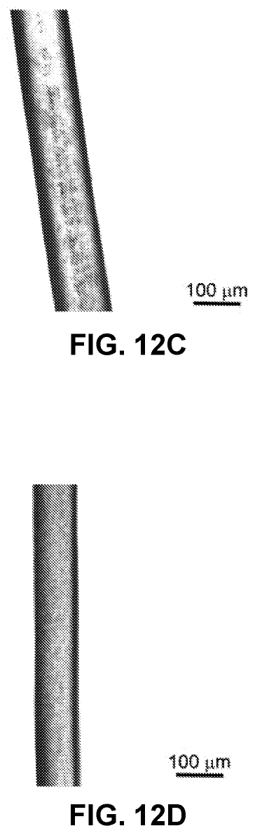

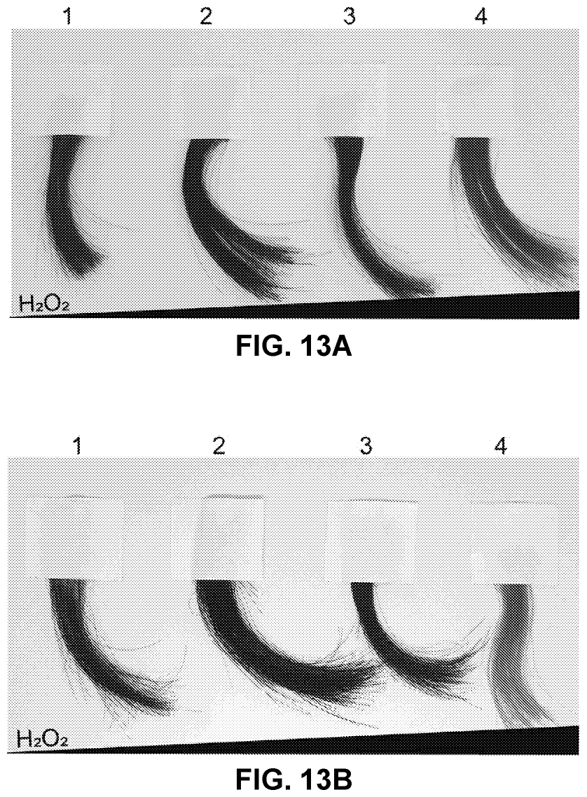

To demonstrate the reproducibility of this method we purchased blond human hair samples from two different vendors (see Material and Methods for description of sources). After 2 hours reaction time, both hair samples treated with the different alkaline conditions (3% NH₄OH, 0.05 N NaOH, and Tris buffer pH 8.5) showed moderate color change (mild darkening) and did not yield a uniform color (FIGS. 2A-2D). This result was confirmed using blond hair samples purchased from a second vendor (FIGS. 8A-8C). As mentioned above, previous studies have used metal ions to fix polydopamine to human hair at room temperature, and this was reproduced for comparison to our own study (FIGS. 2A-20).(24) To eliminate the need for heavy metals such as copper and iron,(35, 36) which are generally considered toxic additives,(25) we employ higher reaction temperatures, which have been shown to enhance the deposition of melanin coatings in other settings,(37) and are used in a salon setting or at home using a hair dryer. Hence, to speed up the process (from day to hours) and to obtain a more uniform melanin deposition, we demonstrate the effect of reaction temperature on hair dyeing. As an initial test, we found that successful reactions were performed in solution at physiological temperature (37-40° C.). UV-Vis spectroscopy was used to monitor the formation of polydopamine in solution at different reaction times and for different reaction conditions at both room temperature (RT) as well as 37-40° C. (FIGS. 2A-2D and 9A-9D). These spectra clearly show an increased intensity of the band around 400 nm (typical of polydopamine) for all polymerization carried out at higher temperature, even at lower reaction time, thereby confirming that the polymerization rate can be enhanced by increasing the temperature. The color of the obtained solutions clearly supports this result as the reaction performed at 37-40° C. yielded visibly darker colors at shorter time periods (FIGS. 10A-10D).

Increased temperatures yielded darker and more uniform hair colors, (FIG. 2B and FIG. 8B) mimicking results obtained using the metal-containing protocol (sample 4 in FIGS. 2A-2C). Different shades of brown color can be better visualized in the optical microscopy images (FIG. 2C, FIG. 1C). Among the conditions tested, hair samples treated with ammonium or sodium hydroxide showed the darkest colors, and the success of the ammonium hydroxide testing was particularly interesting owing to its current use in commercial products. Moreover, these first results suggest that hair darkening can be achieved avoiding the use of H₂O₂, which concentration in permanent hair dyeing protocols is generally around 6%.(38)

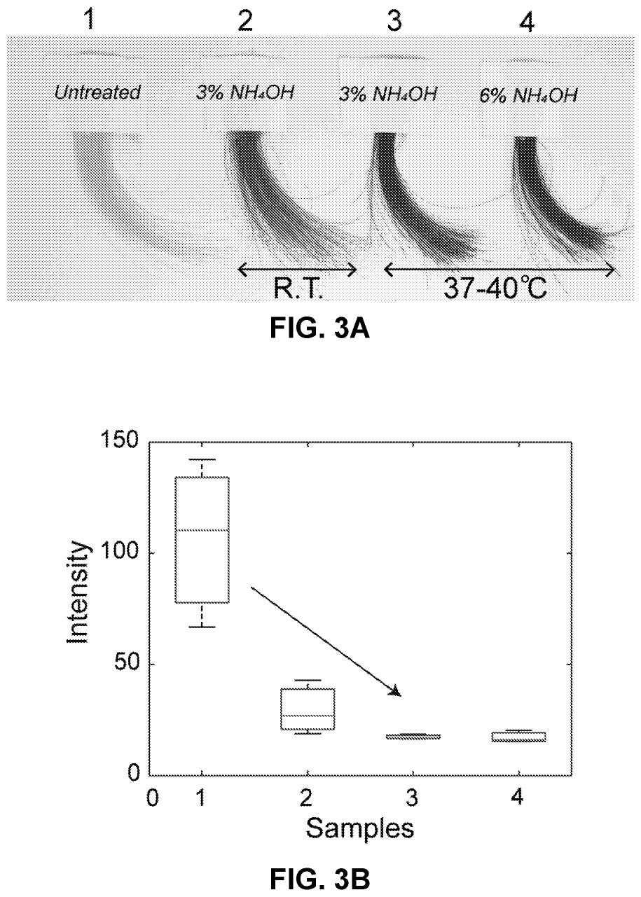

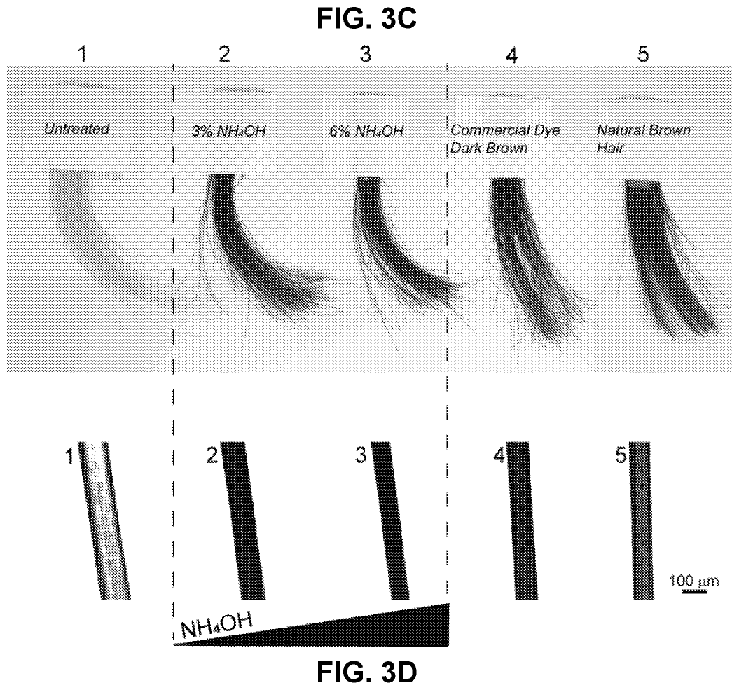

Following initial studies, temperature and ammonium hydroxide concentrations were adjusted to optimize conditions and obtain the various hair colors (FIGS. 3A-3D).

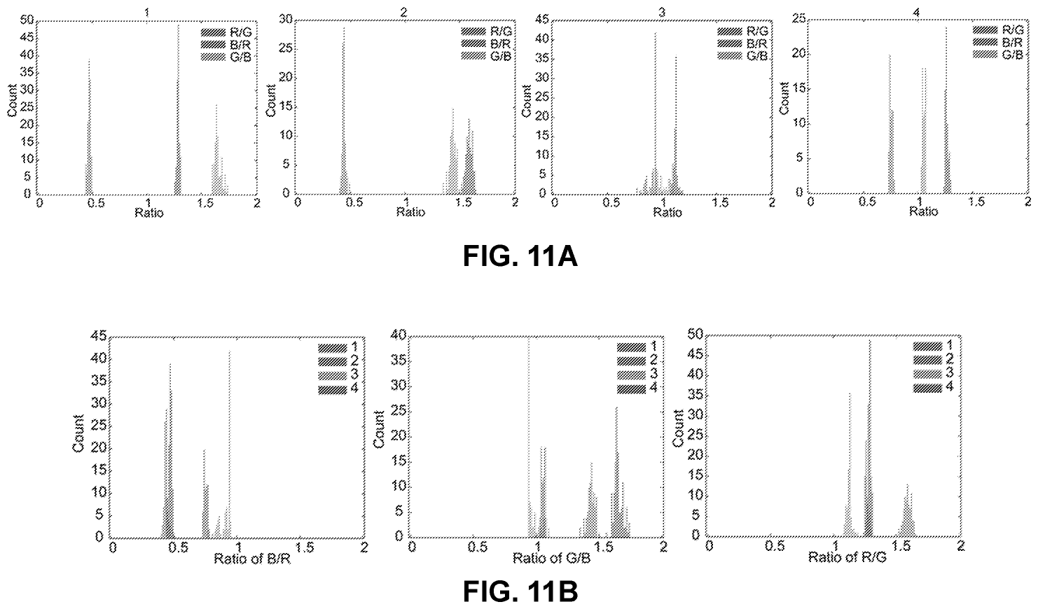

Hair darkening can be observed both by visual inspection (FIG. 3A), as well as by color intensity measurements (FIG. 3B) and analysis of RGB color components (FIGS. 11A-11B). Higher concentrations of ammonium hydroxide in the reaction mixture (6% vs 3%), correlate with slightly darker brown/black colors that resemble natural dark brown hair as well as hair dyed with dark brown commercially available hair dye (FIGS. 3C-3D). We note that 6% ammonium hydroxide is still within the tolerated ammonium hydroxide concentrations, which in case of professional hair dyes, can be above 10%.(34) These results indicate that synthetic melanin can be used successfully as a simple, biomimetic, two-ingredients hair dye without the need for metals or harsh reagents. We employ natural hair pigment mimics and utilize a commercially common reagent, ammonia, albeit at a lower concentration, and negate the need for complex chemical mixtures and H₂O₂.

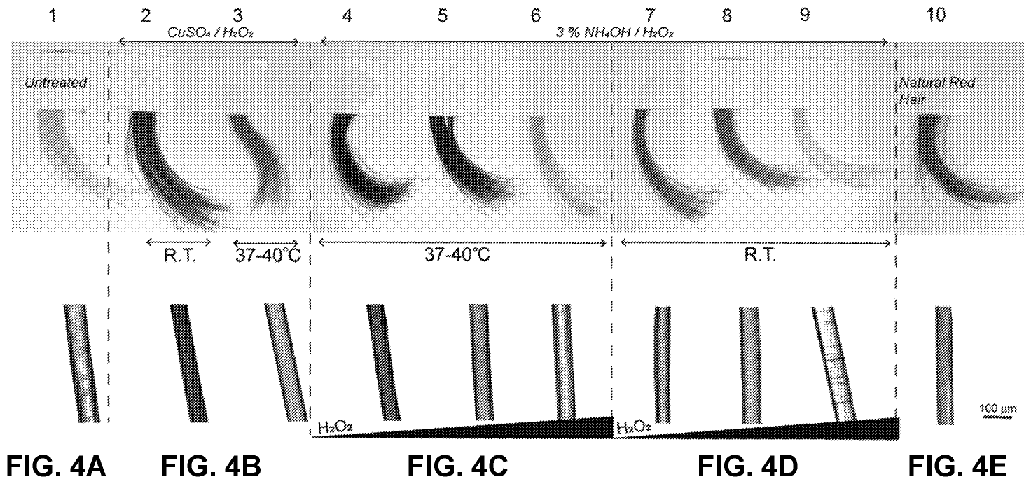

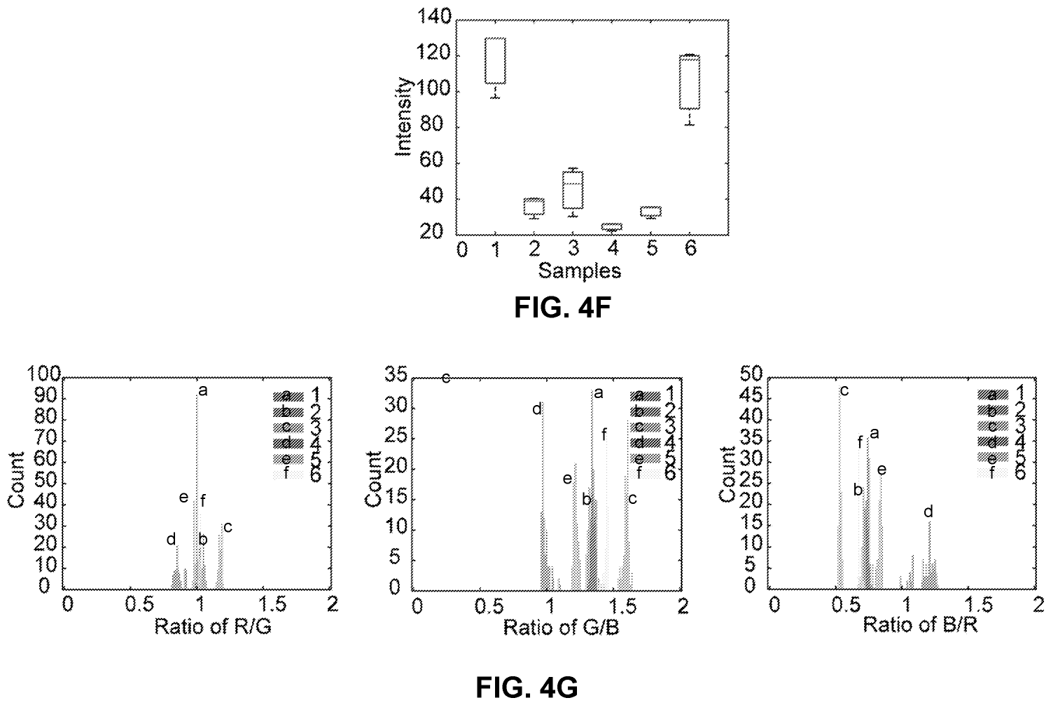

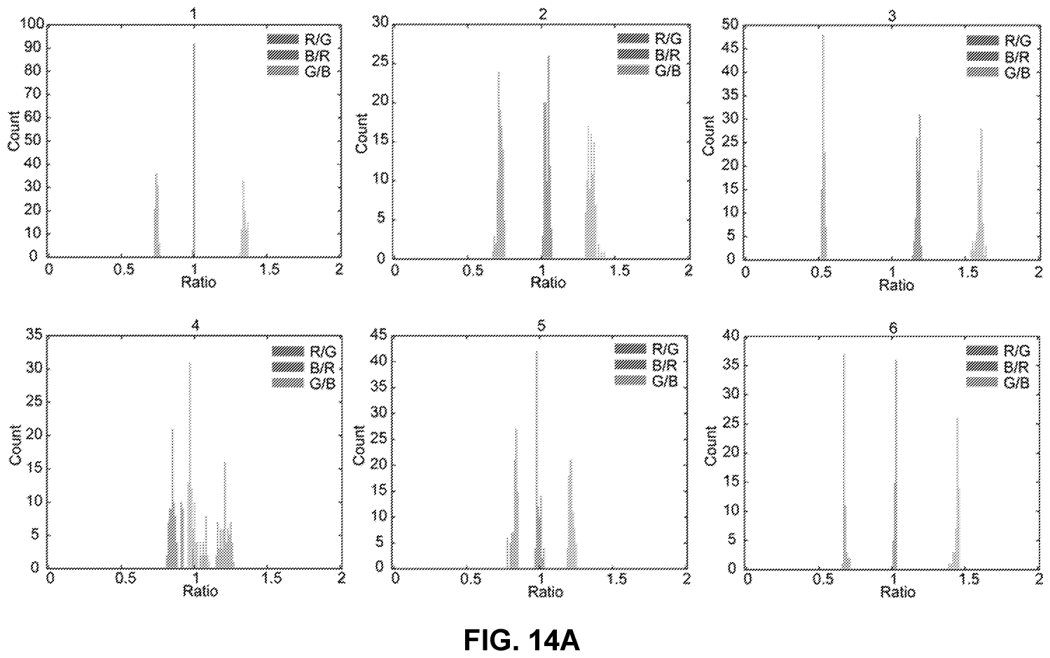

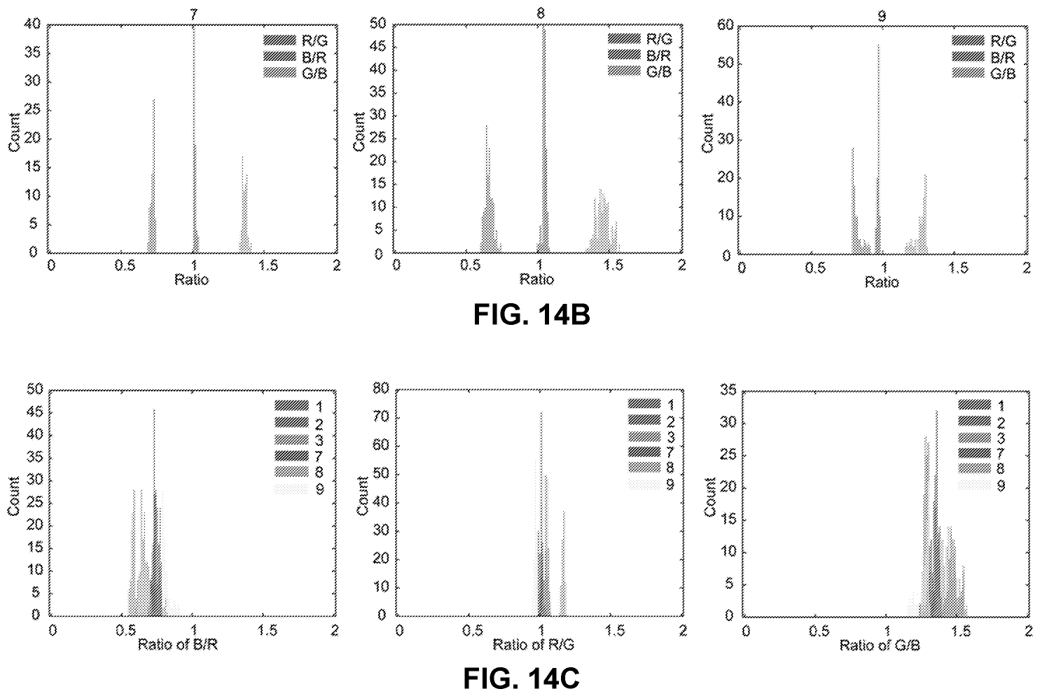

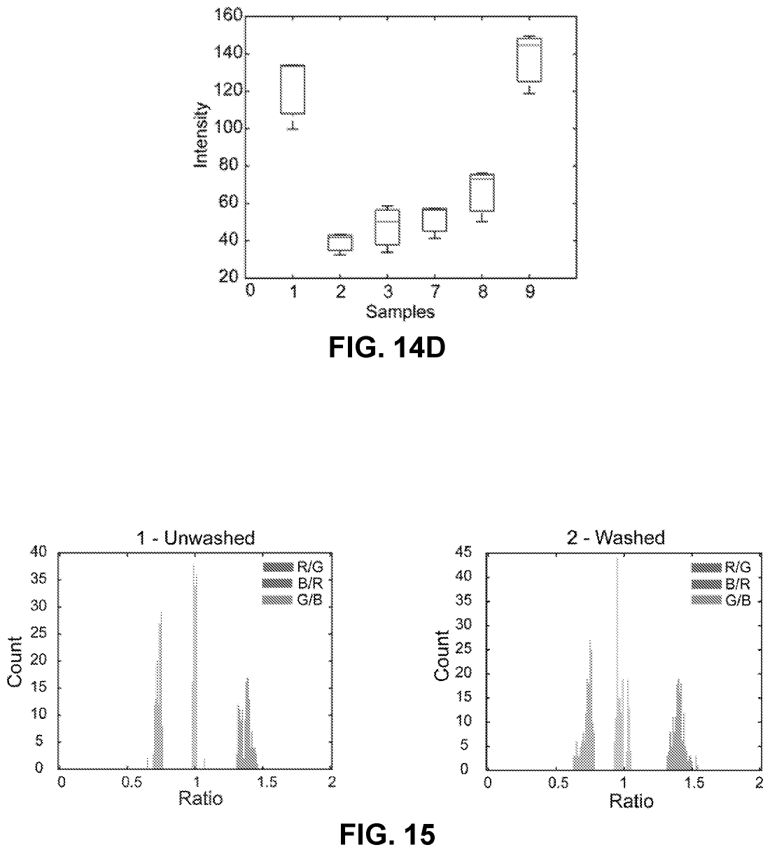

Room temperature comparison studies with CuSO₄/H₂O₂ addition (10 mM CuSO₄, and 15 mM H₂O₂) showed a dark brown coloration (FIGS. 2A-2D). During experimentation, we discovered that these reaction solutions immediately turned red, and that both the use of different reaction temperatures as well as H₂O₂ concentrations (FIGS. 10A-10D) could yield variable brown and red shades. In particular, for longer reaction times, the use of higher concentration of H₂O₂ resulted in richer red-orange colors. When hair samples were dyed using this protocol, higher concentrations of H₂O₂ resulted in a bright orange color (FIGS. 4A-4G, 12A-12D and 13A-13B) resembling that of natural red hair (FIG. 4E). Although addition of copper into the reaction mixture produced the brightest orange color (FIGS. 4B, 4F and 4G), coupling H₂O₂ to the NH₄OH-based protocol also resulted in either dark (37-40° C., FIG. 4C and FIGS. 14A-14D) or light gold shades (room temperature, FIG. 4D and FIGS. 14A-140), as compared to the darker brown colors previously obtained (FIGS. 3A-3D). These results suggest that, together with temperature and base concentrations, H₂O₂ can be added to the formulations up to 3% to obtain a wider range of shades. In particular, H₂O₂ concentrations (<3%, 1 M) lower than those generally employed in permanent hair dyeing protocols (up to 6%, 2 M),(5, 36) can shifts the classic dark brown/black eumelanin color toward clearer and warmer shades.

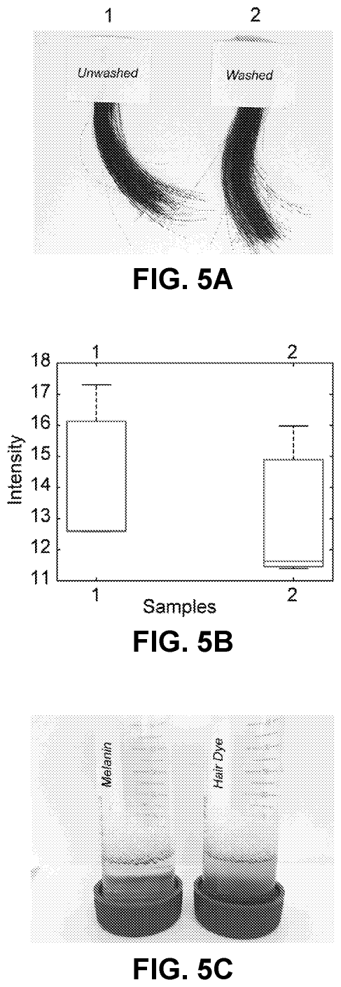

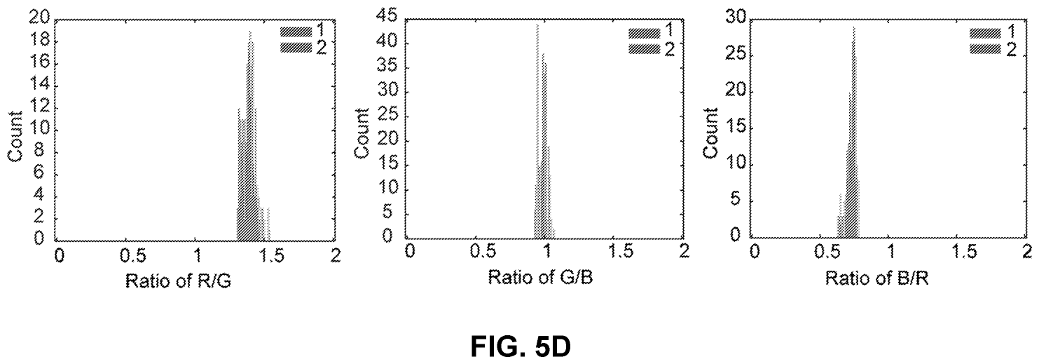

Initially, as noted above, all experiments were carried out using a washing step after dye application: five times with water, and three with shampoo (see Materials and Methods for description). Next, we evaluated the color persistence after a multiple wash protocol. Here, an important distinction needs to be made between semi-permanent and permanent hair dyes. While the former washes away within 4 to 6 weeks of application in the case of washing every other day,(5, 38) the latter is persistent for 6 weeks with washing every other day. Based on a washing schedule of every two days for 5 weeks, we designed a follow up study. This test for color persistence involved 5 wash cycles with water, followed by 18 with shampoo. Specifically, after washing 5 times with water, we applied a 10% shampoo solution to wash the hair samples, and this procedure was repeated 18 separate times (see Materials and Methods for exact protocol). Subsequently, retention of hair color was evaluated by photographic imaging (FIG. 5A) with color intensity/RGB analysis (FIGS. 5B, 5D and FIG. 15).

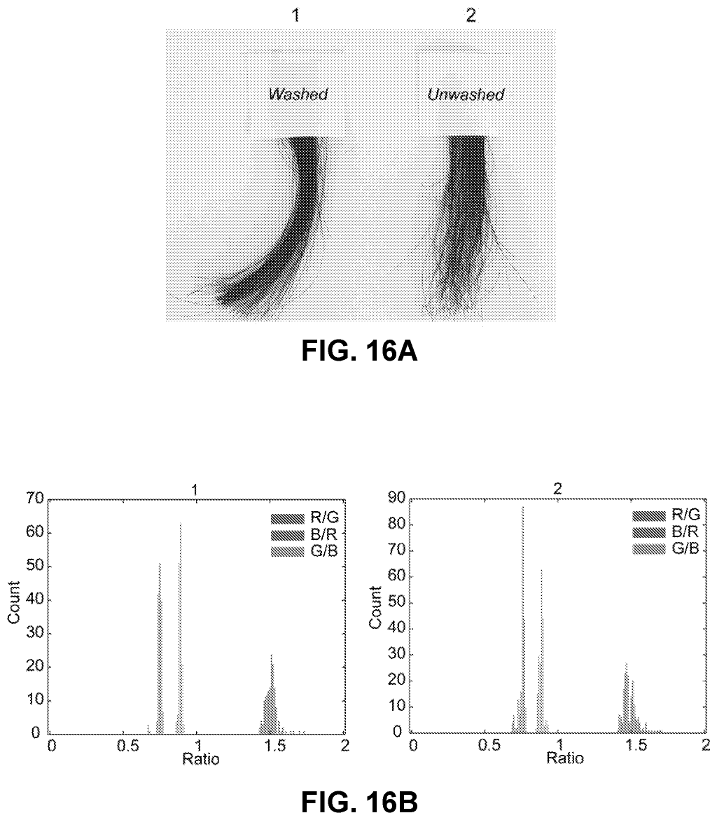

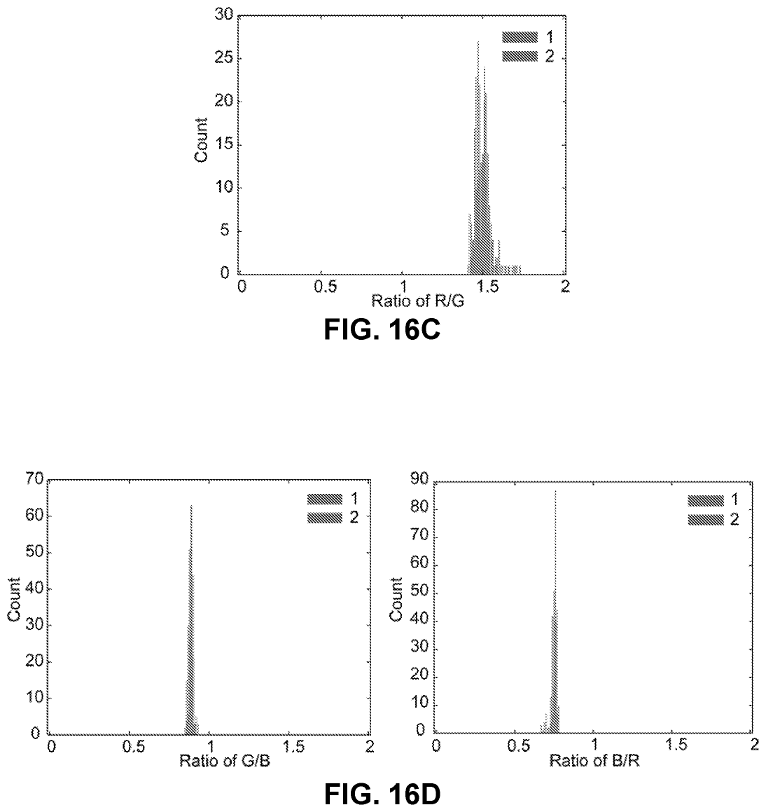

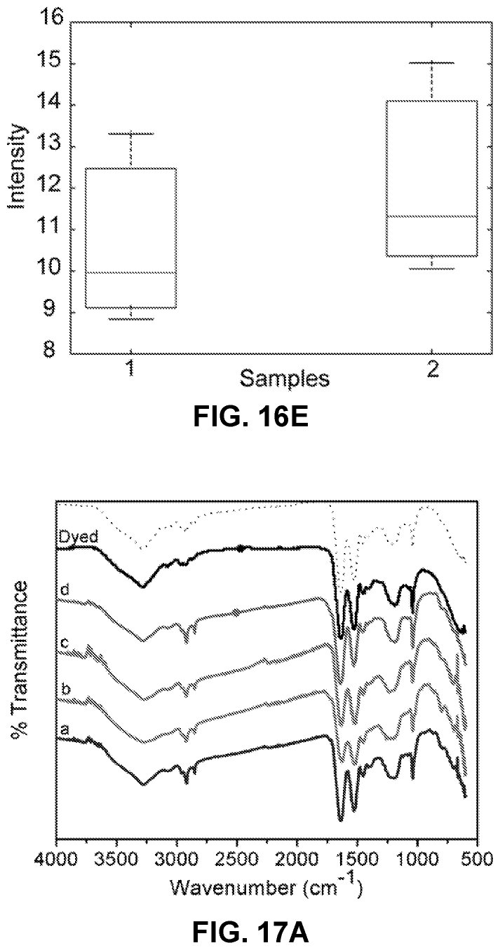

In the initial test, we used hair dyed using dopamine (5 mg/mL) and 6% NH₄OH for 2 hours at 37-40° C. degrees (i.e. conditions used in FIG. 3C, sample 3). These conditions were chosen because, among all the different colors obtained, this one best mimics the color of hair dyed with a commercial, dark brown hair dye (FIG. 3C). The synthetic melanin-based dye resists these washes without any evidence of color change or fading, and the color persistence is similar to that of hair dyed with the permanent, commercially available hair dye (FIGS. 16A-16E). We note that while the soiled solution resulting from washing hair dyed with the commercial dye turned brown, the solution derived from washes of the melanin-dyed hair was colorless (FIG. 5C). These results confirm that our methodology provides a permanent hair dye that persists at least as well as commercial products.

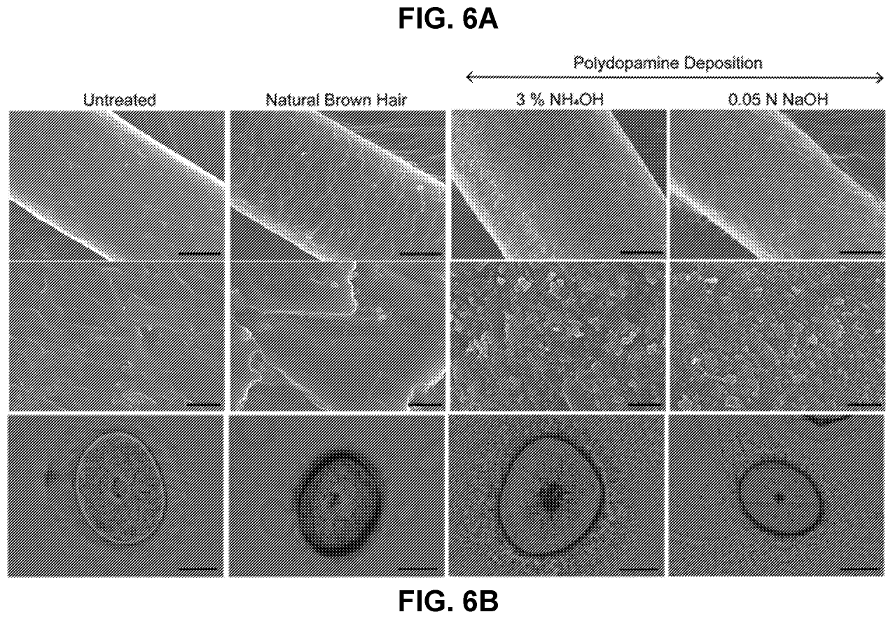

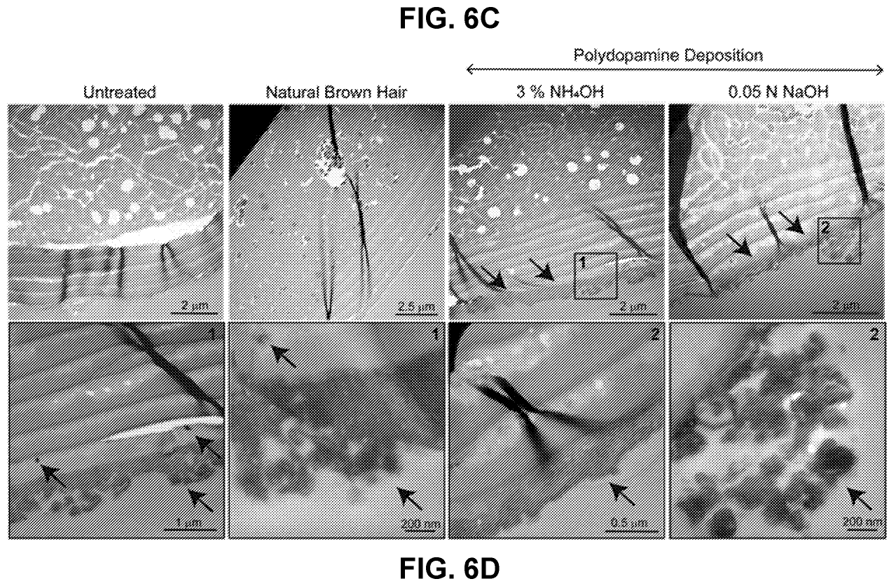

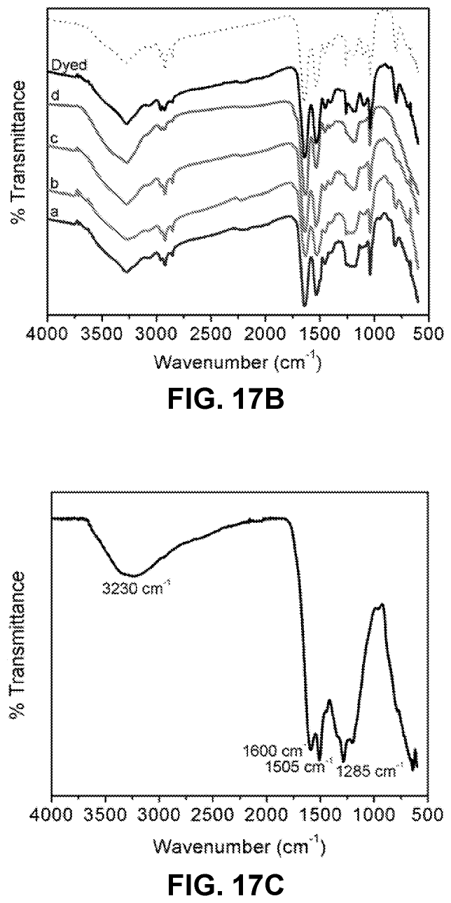

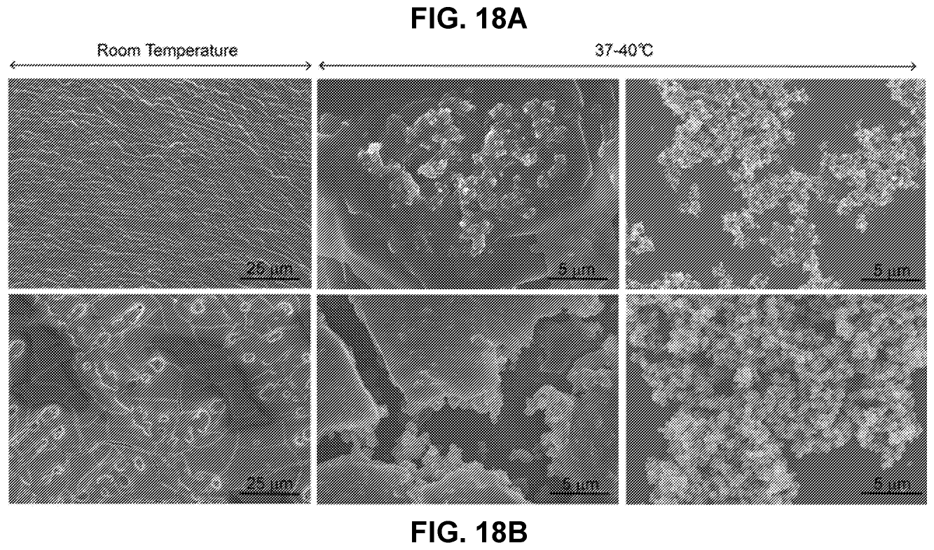

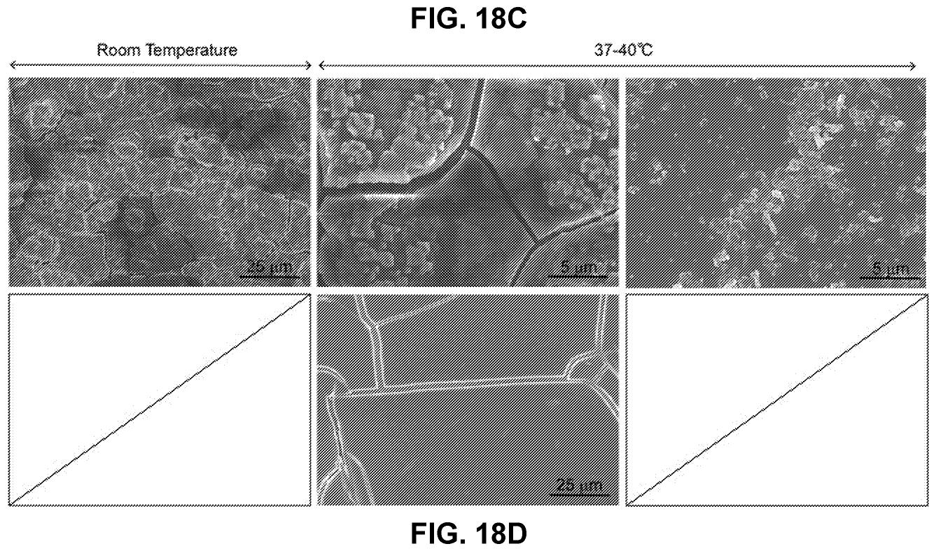

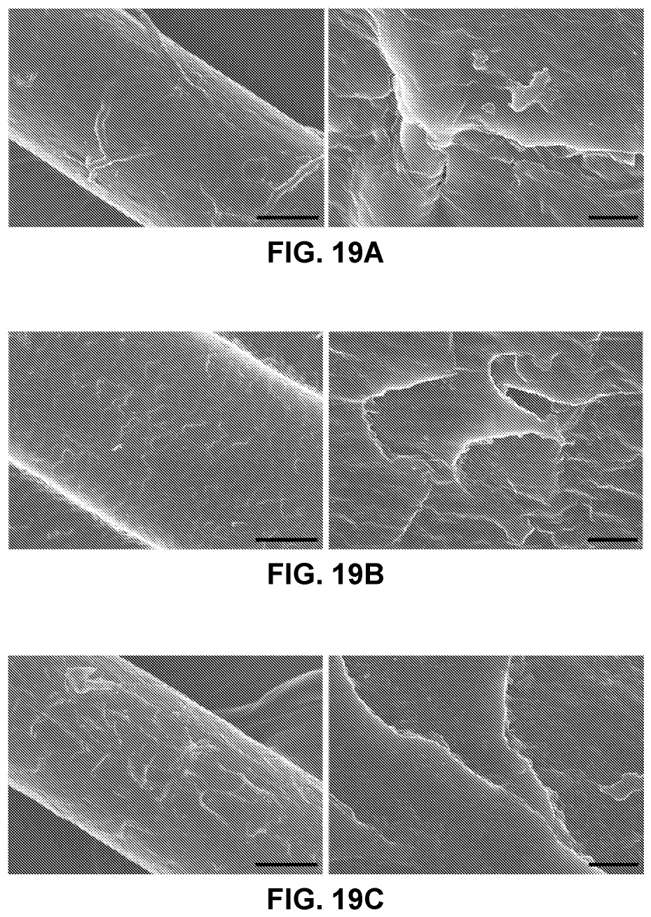

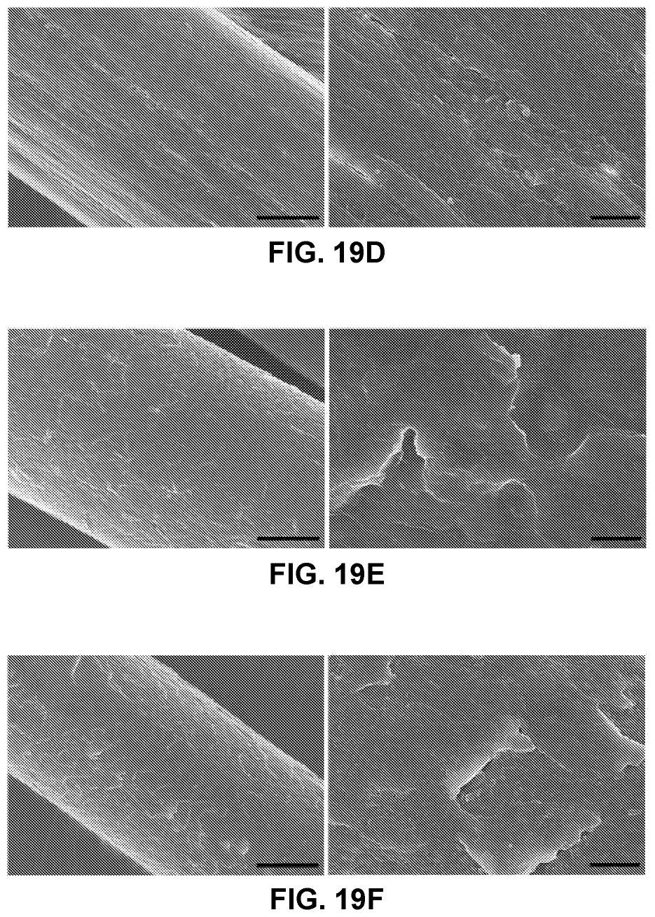

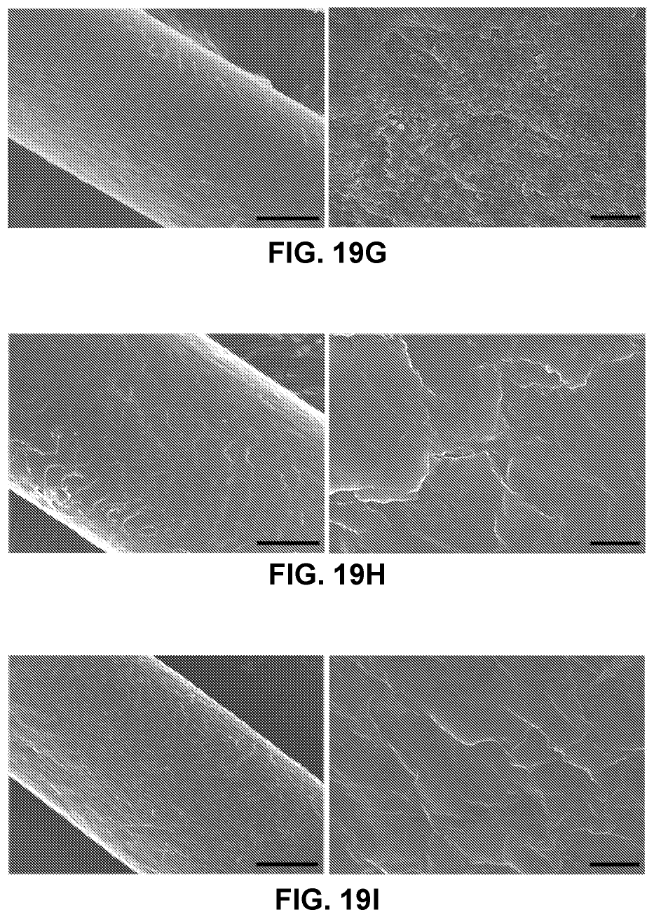

Although dyeing by conventional permanent hair dyes leads to long lasting colors, the harsh conditions employed and the penetration of the small molecule dyes into the cortex, make these methods invasive. Hence, with the next series of experiments we aimed to investigate the mechanism of melanin deposition, its localization within the hair and the morphology of the resulting colored fibers. First, changes in chemical composition at the hair surface was evaluated by FTIR-ATR spectroscopy. This technique mainly probes the surface of hair since the evanescent wave penetrates only a couple of micrometers in depth. Therefore, changes in the typical hair spectra are indicative of changes in the chemical composition of the hair surface. IR spectra of hair samples deriving from two different vendors and dyed with polydopamine under different reaction conditions (FIGS. 17A-17B) revealed only very minor changes, located in the amide I and amide II bands regions (1690-1600 cm⁻¹ and 1575-1480 cm⁻¹, respectively) which derives from overlapping with typical polydopamine bands (FIG. 17C).(22) While these results confirm the presence of polydopamine on the hair surface, insights into the dyeing mechanism can be obtained using multiple imaging techniques (FIGS. 6A-6D). Specifically, SEM analysis suggested that all oxidations carried out under basic conditions (Tris buffer, NH₄OH and NaOH) result in the formation of nanostructures that can be observed before and after (FIGS. 18A-18D) purification of the polydopamine solutions. Oxidation performed at room temperature or using CuSO₄/H₂O₂, did not produce any of these structures. These results support the finding that (i) polydopamine deposition carried out with and without the use of metals occurs via two different mechanisms and that (ii) the formation of melanin nanoparticles obtained using alkaline conditions can be catalyzed by heat. As a result, while hair samples treated with NH₄OH and NaOH were coated by these nanoparticles (FIG. 6A) and (FIGS. 19A-19I), hair treated with CuSO₄/H₂O₂ underwent film deposition (FIGS. 19A-19I).

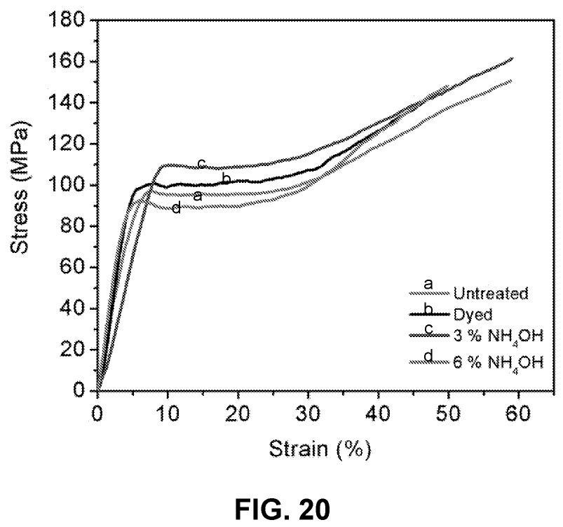

Optical microscopy analysis of cross-sections of hair treated with NH₄OH and NaOH highlighted both a darker color as well as a darker profile (FIG. 6B) as compared to untreated hair. TEM imaging confirmed that this darker profile derives from the nanostructured melanin coatings (FIGS. 6C-6D, black arrows highlight the presence of the synthetic melanin layer). Although a few dark nanostructures resembling those found in natural dark brown hair were observed in the cuticle layers of melanin-coated hair, TEM images confirmed that the dyeing mechanism mainly occurs via nanoparticle deposition without deep penetration into the hair cortex. All together, these results indicate that the proposed protocol allows successful hair dyeing in a permanent manner without compromising the inner hair composition and structure, and more importantly, without altering the mechanical properties of the hair (FIG. 20 and Table 1).

TABLE 1
Mechanical properties of hair before and after dyeing using either 3%
or 6% NH₄OH or using a commercially available hair dye. The average
results are indicated in bold.

| | Strain at Break | Young's Modulus |
|---|---|---|
| Conditions | (mm/mm) | (GPa) |
| Untreated | 0.795 | 3.073 |
| | 0.606 | 3.482 |
| | 0.746 | 1.716 |
| | 0.681 | 2.032 |
| | 0.524 | 3.448 |
| | 0.6704 | 2.7502 |
| 3% NH₄OH* | 0.644 | 1.92 |
| | 0.576 | 2.875 |
| | 0.815 | 1.743 |
| | 0.77 | 2.737 |
| | 0.83 | 1.841 |
| | 0.727 | 2.2232 |
| 6% NH₄OH* | 0.566 | 3.415 |
| | 0.617 | 3.156 |
| | 0.566 | 2.181 |
| | 0.614 | 2.869 |
| | 0.671 | 2.304 |
| | 0.5988 | 2.785 |
| Dyed** | 0.592 | 2.657 |
| | 0.461 | 4.566 |
| | 0.8 | 1.931 |
| | 0.653 | 2.107 |
| | 0.6265 | 2.81525 |

*Hair dyed with polydopamine at 37-40° C. for 2 h.
**Hair dyed using a commercial hair dye.

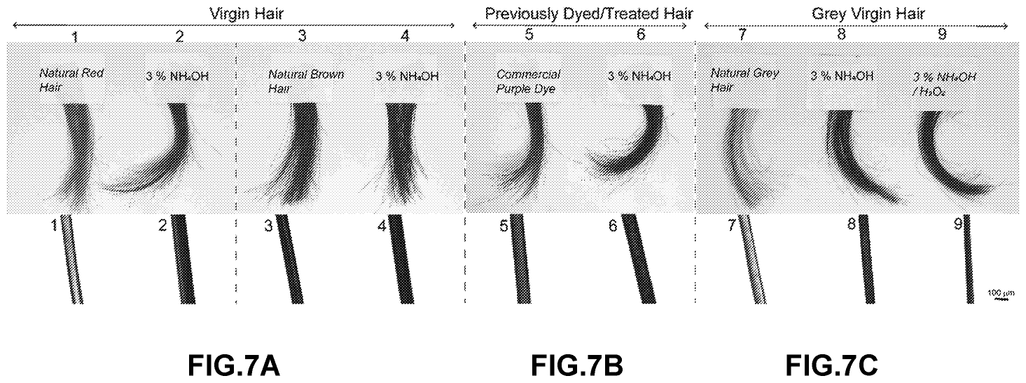

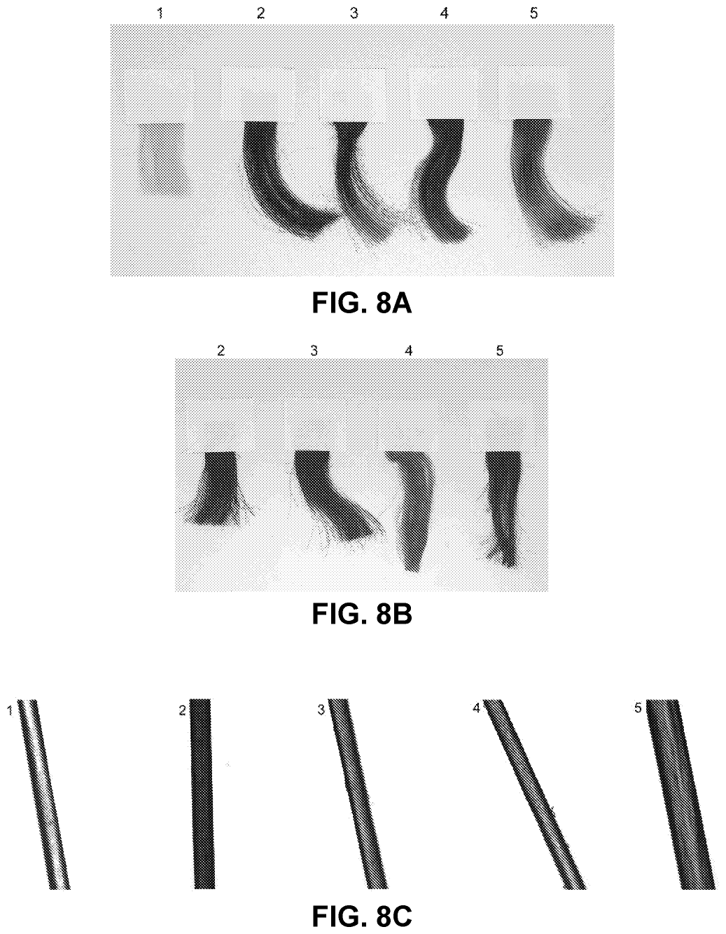

The goal of the last experiment in this Example was to demonstrate that polydopamine deposition can be applied to very diverse hair samples. We aimed to determine if this approach could be considered a universal hair dyeing method. We collected hair samples of four very different colors (natural red, brown, grey and dyed purple) from human donors of different ages and ethnicities. We carried out dopamine polymerization on these human hair samples at 37-40° C. for 2 hours using 3% NH₄OH. Hair photographs and optical microscopy images of hair before and after dying (FIGS. 7A-7C) reveal successful hair darkening for all of the tested samples. The natural brown and red hair (FIG. 7A) had not previously been chemically treated (dyed or bleached). The bright purple hair sample (FIG. 7B) had been previously bleached, and was dyed with a commercial purple hair dye in the two weeks before the experiment. The successful darkening of such different colors and diverse hair samples highlights the efficacy and versatility of this method. Furthermore, the ability to cover naturally grey hair (FIG. 7C) and even to tune the color toward their original warmer brown shades (sample 9, FIG. 7C) by employing H₂O₂-containing protocols (FIGS. 4A-4G), confirms the potential of synthetic melanin as a viable hair dye.

Despite concerns regarding the possible toxicity of commercially available hair dyes, their usage continues to grow, and the lack of modern approaches make this branch of cosmetics a very interesting target for novel and rapidly rising nanomaterial-based approaches. In this work we demonstrate, for the first time, the deposition of synthetic melanin onto human hair without the need for metals, and using similar or milder conditions as compared to generally employed methods used for commercially available hair dyes. This innovative technique allows hair darkening within two hours at physiological temperature (37-40° C.). Increasing concentration of base, resulted in a darker color, whereas the addition of H₂O₂ yielded warmer and orange/gold, natural-looking shades. The resulting colors were comparable to those of hair dyed with commercially available products, and more importantly, they resembled natural hair colors. Morphological studies suggests that synthetic melanin was deposited onto the hair surface in a nanoparticulate form. This colored layer was found to be resistant to at least 18 washes and did not alter the mechanical properties of the hair. These combined results point out the relevance of this novel method, the potential of biomaterials-based approaches in hair and cosmetics, and most importantly, we specifically demonstrate performance arising by engineering systems to perform like natural materials. In this case, employing synthetic melanin as an additive precisely where melanin is naturally used.

Materials and Methods for Example 1:

Polydopamine hydrochloride was obtained from Frontier Scientific, sodium hydroxide and ammonium hydroxide 28-30% (w/v) solution were purchased from Sigma Aldrich. 30% H₂O₂ (w/v) stock solution was purchased from Fisher Scientific. To demonstrate the reproducibility of this method, blond human hair samples were purchased from two different vendors. While most of the experiments were carried out using either blond or dark brown hair obtained from Jerome Krause Fashion Hair (Evanston, Ill.), some experiments were repeated using hair samples purchased from a second vendor (Emosa #613 blond and #2 dark brown) and are reported in the Supplementary Materials. Natural brown, red, and grey hair that had not been dyed or bleached before, and treated, purple-dyed hair were kindly donated. UV-Vis spectroscopy measurements were performed using an Agilent Cary 100 UV-Vis spectrometer using quartz cuvettes. Scanning electron microscopy (SEM) images were acquired on a Hitachi 54800-II cFEG SEM and a Hitachi SU8030, and transmission electron microscopy (TEM) images were acquired on a Hitachi 2300 (scanning TEM) and a JEOL ARM 300 F. Hair samples were imaged using a Leica BM6B widefield optical microscope. FTIR-ATR spectroscopy of both polydopamine and hair samples was performed using a Nexus 870 spectrometer (Thermo Nicolet) and hair mechanical properties were determined using an A. Sintech 20G tensile test machine.

Hair Dyeing. Hair dyeing was carried out using 5 mg/mL monomer (dopamine HCl) in water and hair samples were approximately 2 cm long. The volume of the solution was selected in order to cover the hair sample completely (generally 1 or 2 mL depending on hair size). Either alkaline (Tris buffer pH 8.5 10 mM, 3% or 6% NH₄OH and 0.05 N NaOH) or oxidizing conditions (10 mM CuSO₄ and 100/50/15 mM or 1M H₂O₂) were used for this process. The reaction solutions were stirred either at room temperature or at 37-40° C. After 2 h, hair samples were washed 5 times with water.

Hair Washing. Hair samples were washed with a 10% shampoo solution (Ceramol, Unifarco Biomedical) 3 times and finally rinsed with water prior to imaging/analysis. During each wash, hair was immersed in the shampoo solution and vortex for 30 seconds. To test the persistence of the color after multiple washes, hair samples were washed for an additional 15 times (in total, 5 times with water and 18 with shampoo). Hair color was compared before and after washing.

UV-Vis Spectroscopy of Polydopamine Solutions. UV-Vis time-dependent spectra were recorded by withdrawing 104 of polydopamine solution from each sample at different time intervals. The samples were then diluted in 1 mL water and analysed.

SEM of Polydopamine. After a 2 hour reaction, polydopamine solutions obtained using the above reported conditions were drop casted and evaporated onto a silicon wafer substrate at room temperature. The rest of the sample was centrifuged and the dark-brown precipitate was resuspended in water. This process was repeated three times. All samples were coated with a 6 nm thick-osmium layer and imaged using a Hitachi SU8030 cFEG SEM and a Hitachi SU8030 at 10 kV and 6 kV.

SEM of Hair Samples. Hair samples were adhered onto aluminum SEM stubs by pressing lightly onto carbon tape using a clean glove. The samples were coated with 10 nm osmium and imaged using a Hitachi SU8030 cFEG SEM at 10.0 kV.

Preparation of Hair Samples for TEM. Hair samples were placed in silicon molds in Embed812 resin and polymerized at 65° C. for 48 hours. Ultrathin sections of ca. 80 nm thickness were obtained with an ultramicrotome (Ultracut-S, Leica) and a diamond knife (Diatome). Sections were placed on copper mesh grids, or on slotted copper grids with a formvar/carbon film (EMS).

Preparation of Hair Cross-Sections for Optical Microscopy. Hair was embedded with optimal cutting temperature (OCT) medium and 10 μm sections were cryosectioned at −20° C. and deposited onto glass microscope slides.

Mechanical Properties of Hair. Hair samples with a diameter between 75 and 105 μm were used for this study. Stress-strain curves are reported as an average of five different measurements.

RGB Color Analysis of Hair Photographs. MatLab software was used to determine RGB color components and intensities in order to differentiate the color of hair dyed using different conditions as well as to investigate color fading after multiple washes.

### REFERENCES CORRESPONDING TO EXAMPLE 1

1. M. V. R. Velasco et al., Hair fiber characteristics and methods to evaluate hair physical and mechanical properties. Braz J Pharm Sci 45, 153-162 (2009).
2. S. Ghiani et al., Characterization of human hair melanin and its degradation products by means of magnetic resonance techniques. Magn Reson Chem 46, 471-479 (2008).
3. D. Van Neste, D. J. Tobin, Hair cycle and hair pigmentation: dynamic interactions and changes associated with aging. Micron 35, 193-200 (2004).
4. O. J. X. Morel, R. M. Christie, Current Trends in the Chemistry of Permanent Hair Dyeing. Chem Rev 111, 2537-2561 (2011).
5. A. Guerra-Tapia, E. Gonzalez-Guerra, Hair cosmetics: dyes. Actas Dermosifiliogr 105, 833-839 (2014).
6. E. K. Nishimura, S. R. Granter, D. E. Fisher, Mechanisms of hair graying: Incomplete melanocyte stem cell maintenance in the niche. Science 307, 720-724 (2005).
7. F. Ricci, C. De Simone, L. Del Regno, K. Peris, Drug-induced hair colour changes. Eur J Dermatol 26, 531-536 (2016).
8. E. V. Keogh, R. J. Walsh, Rate of Greying of Human Hair. Nature 207, 877-& (1965).
9. M. I. Quiroga, [History of cosmetics]. Dia Med 25, 1300-1306 (1953).
10. S. Kumar, Exploratory analysis of global cosmetic industry: major players, technology and market trends. Technovation 25, 1263-1272 (2005).
11. D. Patel, S. Narayana, B. Krishnaswamy, Trends in use of hair dye: a cross-sectional study. Int J Trichology 5, 140-143 (2013).
12. H. Sosted, U. Hesse, T. Menne, K. E. Andersen, J. D. Johansen, Contact dermatitis to hair dyes in a Danish adult population: an interview-based study. Brit J Dermatol 153, 132-135 (2005).
13. A. Bernard et al., Consumption of hair dye products by the French women population: Usage pattern and exposure assessment. Food and Chemical Toxicology 88, 123-132 (2016).
14. J. P. McFadden et al., Allergy to hair dye—Its incidence is rising, as more and younger people dye their hair. Brit Med J 334, 220-220 (2007).
15. T. B. Zanoni et al., Allergens of permanent hair dyes induces epidermal damage, skin barrier loss and IL-1 alpha increase in epidermal in vitro model. Food Chem Toxicol 112, 265-272 (2018).
16. M. Gago-Dominguez, J. E. Castelao, J. M. Yuan, M. C. Yu, R. K. Ross, Use of permanent hair dyes and bladder-cancer risk. Int J Cancer 91, 575-579 (2001).
17. K. T. Chung, Azo dyes and human health: A review. J Environ Sci Health C Environ Carcinog Ecotoxicol Rev 34, 233-261 (2016).
18. B. Takkouche, M. Etminan, A. Montes-Martinez, Personal use of hair dyes and risk of cancer—A meta-analysis. Jama—J Am Med Assoc 293, 2516-2525 (2005).
19. P. Walter et al., Early use of PbS nanotechnology for an ancient hair dyeing formula. Nano Lett 6, 2215-2219 (2006).
20. S. D. Haveli et al., Hair Fiber as a Nanoreactor in Controlled Synthesis of Fluorescent Gold Nanoparticles. Nano Lett 12, 6212-6217 (2012).
21. C. Luo, L. Y. Zhou, K. Chiou, J. X. Huang, Multifunctional Graphene Hair Dye. Chem-Us 4, 784-794 (2018).
22. Y. L. Liu, K. L. Ai, L. H. Lu, Polydopamine and Its Derivative Materials: Synthesis and Promising Applications in Energy, Environmental, and Biomedical Fields. Chem Rev 114, 5057-5115 (2014).
23. K. M. Im, T. W. Kim, J. R. Jeon, Metal-Chelation-Assisted Deposition of Polydopamine on Human Hair: A Ready-to-Use Eumelanin-Based Hair Dyeing Methodology. Acs Biomater Sci Eng 3, 628-636 (2017).
24. Z. F. Gao, X. Y. Wang, J. B. Gao, F. Xia, Rapid preparation of polydopamine coating as a multifunctional hair dye. Rsc Adv 9, 20492-20496 (2019).
25. S. Borowska, M. M. Brzoska, Metals in cosmetics: implications for human health. J Appl Toxicol 35, 551-572 (2015).
26. Y. Dong, Y. Qiu, D. Gao, K. Zhang, K. Zhou, H. Yin, G. Yi, J. Li, Z. Xia, Q. Fu, Melanin-mimetic multicolor and low-toxicity hair dye. RSC Adv 9, 33617 (2019).
27. E. M. Lent, L. C. B. Crouse, W. S. Eck, Acute and subacute oral toxicity of periodate salts in rats. Regul Toxicol Pharmacol 83, 23-37 (2017).
28. A. Korner, J. Pawelek, Mammalian tyrosinase catalyzes three reactions in the biosynthesis of melanin. Science 217, 1163-1165 (1982).
29. P. A. Riley, Melanin. Int J Biochem Cell B 29, 1235-1239 (1997).
30. J. Liebscher et al., Structure of polydopamine: a never-ending story? Langmuir 29, 10539-10548 (2013).
31. K. Y. Ju, Y. Lee, S. Lee, S. B. Park, J. K. Lee, Bioinspired Polymerization of Dopamine to Generate Melanin-Like Nanoparticles Having an Excellent Free-Radical-Scavenging Property. Biomacromolecules 12, 625-632 (2011).
32. Y. R. Huang et al., Mimicking Melanosomes: Polydopamine Nanoparticles as Artificial Microparasols. Acs Central Sci 3, 564-569 (2017).
33. J. A. Seo et al., Hydrogen peroxide and monoethanolamine are the key causative ingredients for hair dye-induced dermatitis and hair loss. J Dermatol Sci 66, 12-19 (2012).
34. W. Johnson, Safety Assessment of Ammonia and Ammonium Hydroxide as Used in Cosmetics, Cosmetic Ingredient Review, (2017).
35. J. Y. Zhu et al., A rapid deposition of polydopamine coatings induced by iron (III) chloride/hydrogen peroxide for loose nanofiltration. J Colloid Interf Sci 523, 86-97 (2018).
36. C. Zhang et al., CuSO₄/H₂O₂-Induced Rapid Deposition of Polydopamine Coatings with High Uniformity and Enhanced Stability. Angew Chem Int Ed Engl 55, 3054-3057 (2016).
37. P. Zhou et al., Rapidly-Deposited Polydopamine Coating via High Temperature and Vigorous Stirring: Formation, Characterization and Biofunctional Evaluation. Plos One 9, (2014).
38. S. Harrison, R. Sinclair, Hair colouring, permanent styling and hair structure. J Cosmet Dermatol 2, 180-185 (2003).

### Example 2: Control of Hair Coloring Using Artificial Melanin Materials

The composition and properties of hair treated by the present methods, such as color, are dependent on a number of factors including the starting hair color and type, the treatment conditions including temperature and solution composition, and the composition and properties of the artificial melanin materials used. Accordingly, selective adjustment of these parameters provides a means of controlling the composition and properties of hair treated. The capability of the invention to provide selectable control over hair color is described for wide a range of human hair samples and treatment conditions.

FIG. 21 provides a Table summarizing the starting materials and resulting hair color for a range of hair treatment methods and conditions. As shown in FIG. 21, the following treatment conditions were selectively adjusted to provide control of the color of the resultant hair:
Starting hair color and type—Blond, Brown, Red, Gray, Previously dyed; e.g., purple.
Temperature—Room Temperature (≈18° C.) vs. Elevated Temperature (37-40° C.);
Solution Composition—NH₄OH (3%, 6%), NaOH, Tris buffer, H₂O₂ (0.045%, 0.15%, 0.3% and 3%); CuSO₄ (10 mM); and
Environment—In the presence of air.

The left most column of the Table in FIG. 21 indicates the following transformations that were observed—(i) brown/black resulting hair from treated blond hair; (ii) orange/black resulting hair from treated blond hair, (iii) orange/black resulting hair from treated blond hair, (iv) treated brown hair, (v) treated red hair, (vi) treated previously dyed (purple) hair and (vii) treated gray hair. The left most column also provides reference to supporting Figures providing examples of the treated hair. The right most column provides a characterization of the color of the resulting hair upon treatment which ranged from dark blond, brown, dark blond, dark brown, dark grey/light brown, brown with red shades, dark orange, orange (natural red), brown with orange shades, bright blond, orange/gold, blond/gold, light blond, and dark brown with red shades.

As shown in FIG. 21 the present treatment methods are useful for coloring hair. Treatment conditions using a base (e.g., NH₄OH, NaOH, Tris buffer) at elevated temperatures resulted in darkening hair color wherein increasing the concentration of base results in darker color. Treatment conditions including H₂O₂ at different concentrations provides a means of tuning the color of the resulting hair, for example, resulting in light orange to dark brown/red colors. Treatment at elevated temperature resulting in persistent change in hair color resistance to washing and did not significantly alter the hair mechanical properties.

Treatment using NH₄OH in the presence of air at elevated temperature under some conditions suggested that synthetic melanin is deposited on the hair surface in a nanoparticulate form. For the oxidation in the presence of air, H₂O₂ is not required for formation of artificial melanin and deposition on to the hair. It was observed, however, that treatment in the presence of H₂O₂ under some conditions gives rise to red and/or gold color of the treated hair. Under certain conditions treatment in using NH₄OH and H₂O₂ in the presence of air at elevated temperature provided coloring to obtain orange and/or gold shades without requiring metals (such as CuSO₄). These results demonstrates good performance of metal free methods for treatment of hair.

For treatment of including conditions of CuSO₄ and H₂O₂ the formation of a film on the treated hair is observed. Under certain conditions, the metal reacts with H₂O₂ via a Fenton like reaction therefore producing OH radicals and OH— which may function as oxidants for the formation of the artificial melanin.

### Example 3: Bioinspired Chemoenzymatic Route to Artificial Melanin for Hair Pigmentation

Recent reports suggest that next-generation hair dyes might take inspiration from the natural pigment melanin. In humans, melanin imparts color to hair and skin and acts as a natural sunscreen and radical scavenger, thereby protecting lipids and proteins from damage. The most commonly employed synthetic mimic of melanin is polydopamine, and its successful deposition on human hair was recently reported. Herein, we describe an enzymatic approach to synthetic melanin for dyeing human hair in a process that closely mimics part of natural melanogenesis. This chemoenzymatic method avoids the addition of base and enables the implementation of several monomers beyond dopamine, including tyrosine, tyramine, and their derivatives. Advantageously, the enzyme provides a milder process for producing coated hair fibers than conventional chemical hair dyeing methods. In addition to providing natural coloration, these coatings have the potential to act as protective sunscreens that prevent photodamage of the inner hair fibers during exposure to sunlight. The protocols developed herein represent a mild and efficient route to nature-inspired multifunctional coatings. Such materials are promising candidates for artificial hair pigmentation and, more generally, could find extensive application as fiber coatings.

In nature, melanin can be found across various species as a series of different structural forms.¹ Eumelanin, the most common form of melanin in humans, is a dark brown or black pigment found ubiquitously in hair and skin.² Loss of this pigment in hair leads to hair whitening.³ Although synthetic hair dyes are routinely employed to conceal whitening, concerns are mounting regarding potential toxicity and allergies arising from their extensive use.⁴⁻¹² Among the proposed polymeric alternatives to mimic melanin,¹³⁻¹⁶ recently, polydopamine (PDA) has been proposed as a melanin mimetic hair dye.¹⁷⁻²⁰ In our latest work, we demonstrated that spontaneous dopamine oxidation under basic conditions leads to successful deposition of PDA nanoparticles onto human hair.²⁰ Fine-tuning the reaction conditions also enabled the selection of color from a palette ranging from blond to black, with red, orange and brown shades. The resulting nanoparticle coating resisted multiple washes and did not affect mechanical properties of the hair. We demonstrated that this novel, bioinspired hair dye can be applied using basic conditions, which are comparable to those normally used in conventional hair dye protocols,²¹ and that these conditions are milder than previously reported methods for generating PDA coatings.^{17,18,19}

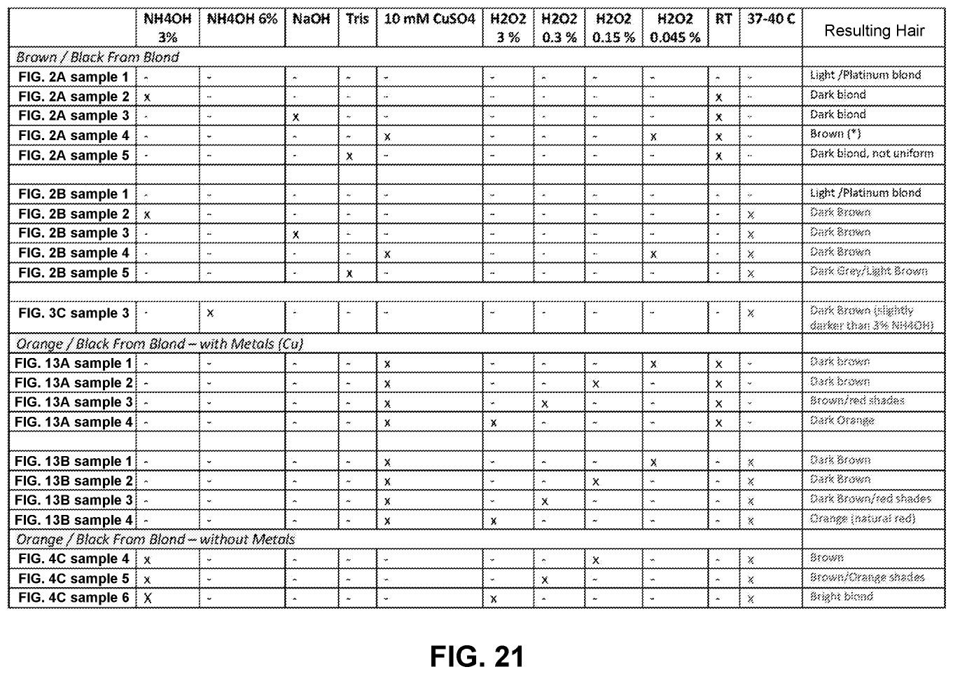

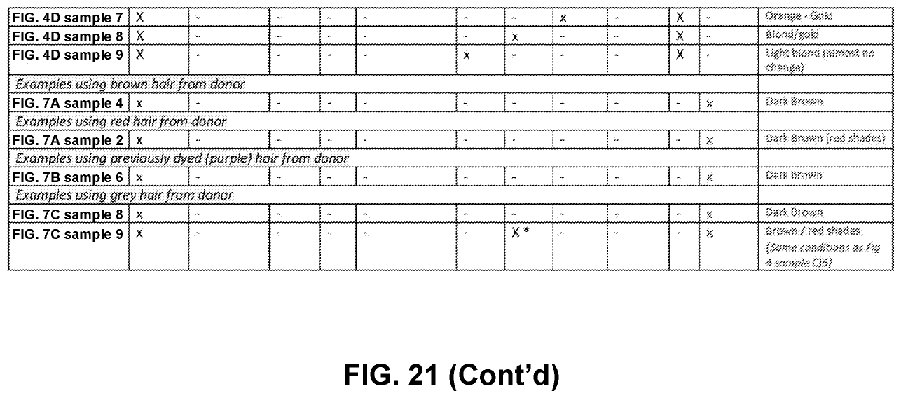

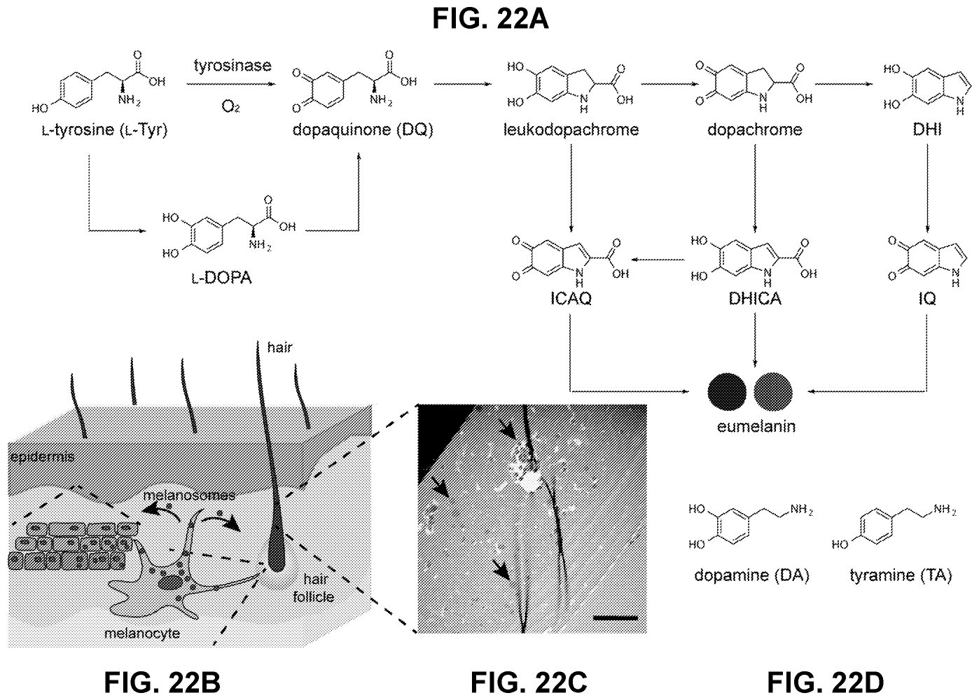

Advancing this work, we next targeted the development of a chemoenzymatic process, with the goal of identifying even milder conditions and of expanding the monomer-substrate scope. To that end, we took inspiration from the biosynthesis of natural melanin. In humans, the biosynthesis of melanin begins with tyrosinase-mediated oxidation of L-Tyr to 3,4-dihydroxyphenylalanine (L-DOPA).²²⁻²⁴ This intermediate undergoes several oxidation reactions leading to polymerization, yielding the dark brown/black eumelanin pigment (FIGS. 22A-22D). This process of melanogenesis occurs inside melanocytes in specialized organelles called melanosomes (FIG. 22B). The resulting dark melanin nanoparticles are then transferred within the hair and epidermis, where they function as a natural, multifunctional dye (FIGS. 22B-22C).^{22,23}

Herein, we describe the use of Agaricus bisporus tyrosinase (EC 1.14.18.1) for the production of synthetic melanin coatings and demonstrate that chemoenzymatically-driven oxidation of dopamine (DA) and its precursors or derivatives leads to successful deposition of synthetic melanin onto human hair. While not broadly applied, large-scale production has been developed to prepare tyrosinase, which finds application in multiple industrial settings, including food processing.²⁵⁻³⁰ Recently, enzymatic production of melanin precursors, with applications in hair dye formulations and beyond, has been described.³¹ We reasoned that if such a chemoenzymatic approach were exploited to deposit synthetic melanin onto hair fibers, it would provide not only a milder, but also a valuable alternative to previously described methods.¹⁷⁻²⁰ This approach offers four advantages over previously reported reaction conditions. First, it avoids the use of basic environments commonly used in hair dye formulations (e.g. ammonia and monoethanolamine).^{20,21,32} Such basic environments can cause skin irritation and damage hair cuticles, especially after repeated applications.^{32,33} Second, its precursors are not as toxic as currently used hair dye chemicals, which include phenol derivatives, resorcinol, and paraphenylenediamine (PPD).¹¹ Third, chemoenzymatic oxidation allows the use of monomer substrates other than dopamine and its derivatives, including natural phenolamines, catecholamines and amino acids, that are safe, scalable and can give rise to a wider range of colors. Finally, enzymatic treatment might facilitate cosmetic translation of this approach as some of these components are already used in the field.

In nature, apart from coloring the surrounding tissues and fibers, natural melanin also acts as a radical scavenger and a natural UV sunscreen.^{23,34} As a broad-range UV light absorber, melanin attenuates solar radiation, an effect partially responsible for protecting the surrounding lipids and proteins of skin and hair. In addition, it is protective as it functions to quench radicals produced by incident radiation.³⁴⁻³⁷ While damage initially takes place in outer hair cuticles, prolonged UV exposure can lead to melanin disruption and subsequent changes in hair color.^{37,38} As a result, sunscreens have been developed to protect skin and hair from intense and prolonged UV irradiation.^{37,39} However, to guarantee efficacy against prolonged exposure, repetitive and uniform application of such conventional formulations are typically required. Herein, we show that synthetic melanin coatings produced via both basic and chemoenzymatic oxidative polymerization have the potential to reinforce natural hair photoprotection by acting as an additional barrier against UV penetration.

Taken together, we demonstrate that chemoenzymatic melanogenesis and deposition on hair provides both a novel and effective path to hair dyeing. This method, using milder conditions and employing a variety of monomers, yields multifunctional hair coatings and expands access to engineered coated fibers through an in situ biotransformation strategy.

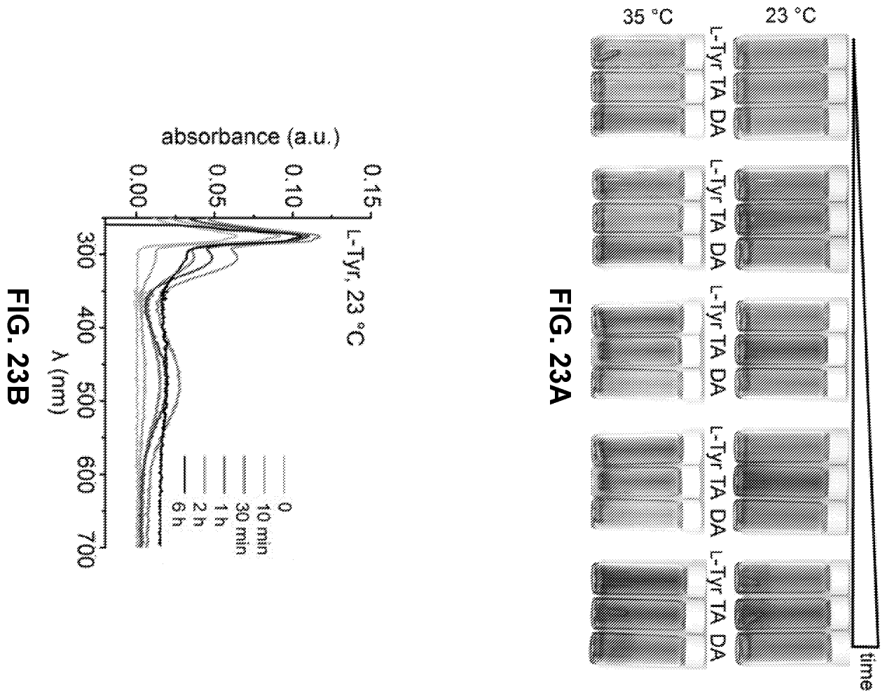

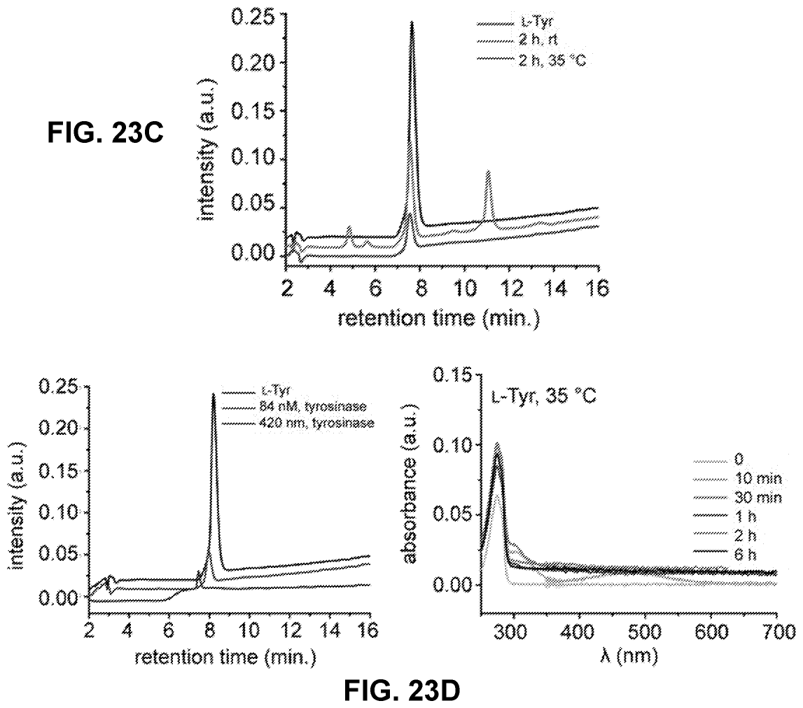

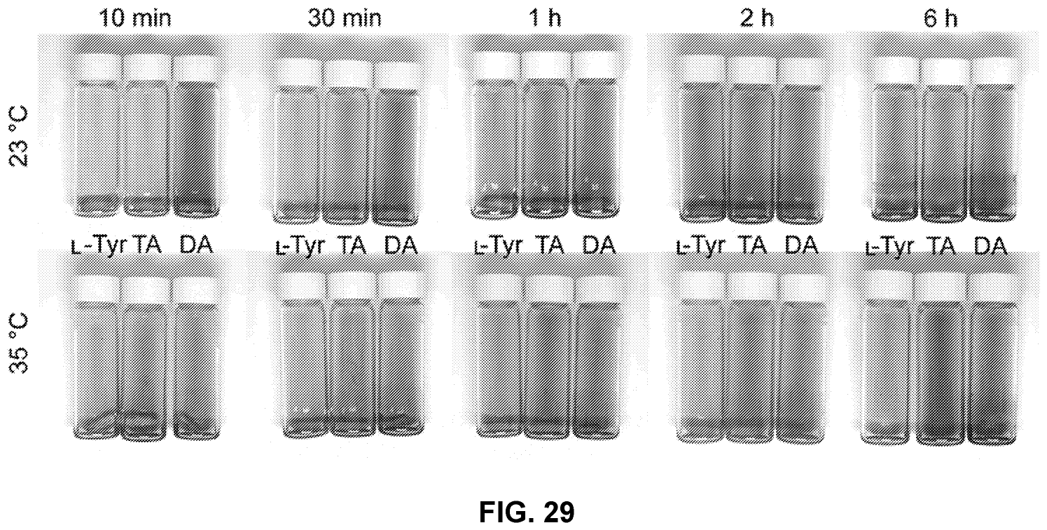

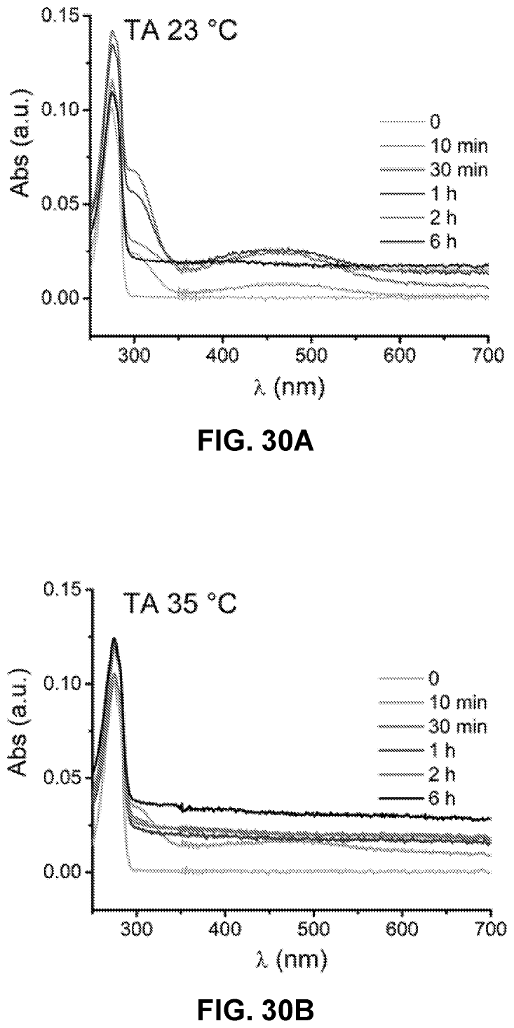

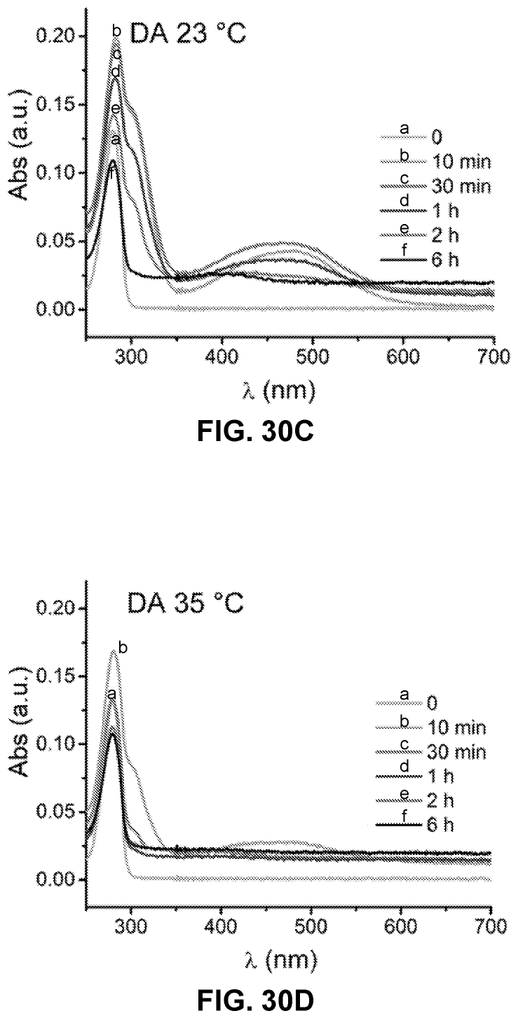

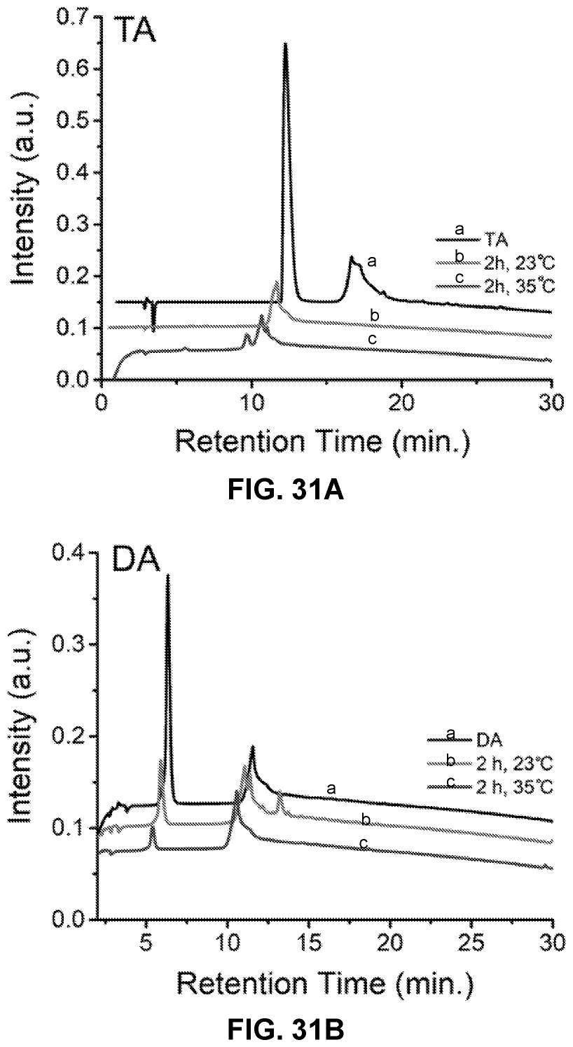

The use of chemoenzymatic oxidative polymerization to produce multifunctional hair coatings has two main advantages. First, it is performed at neutral pH, avoiding the need for basic additives such as NH₄OH. Second, it allows the use of substrates other than DA, such as phenolamines and amino acids. Mushroom tyrosinase has recently been used to promote polydopamine-based film deposition⁴⁰ and nanoparticles formation⁴¹ starting from dopamine as well as natural phenolamines. Although all of the investigated monomers are viable tyrosinase substrates, the 6 h+ reaction time required to deposit films exceeds common hair dyeing applications.⁴⁰ Therefore, as a proof-of-concept, we chose three example monomers, L-Tyr, TA, and DA (FIGS. 22A and 22D), and optimized reaction conditions to synthesize and deposit functional synthetic melanin in a faster and more efficient manner (FIGS. 23A-23D). Aliquots of 2.8 mM Tyr, 3.6 mM TA, or 2.6 mM DA (0.5 mg/mL) monomer solutions were incubated with 84 nM tyrosinase (10 U/mL) at rt and stirred for 6 h.⁴⁰ Samples were withdrawn and analyzed by UV-Vis at different time intervals. As previously reported,⁴⁰ a 6 h reaction time was necessary to obtain effective darkening of the reaction solutions, performed either in water or Dulbecco's phosphate-buffered saline (DPBS) buffer (FIGS. 23A and 29). Across all tested monomers (L-Tyr, TA, DA), color darkening was associated with an initial increase, then decrease in UV-Vis absorption at 310 nm and 475 nm (FIGS. 23B and 30A-30D). A decrease in these signals, deriving from quinone and cyclic amine species respectively,⁴⁰ after the 6 h reaction is indicative of successful polymerization and deposition.⁴⁰ We next performed these reactions at 35° C. and at neutral pH, optimal reaction parameters for mushroom tyrosinase.⁴² This led to a faster process with solutions turning brown/black several minutes after reaction initiation (FIG. 23A). The same effect was observed for reactions carried out in both water and DPBS across all tested monomers (FIG. 29). This was confirmed by the rapid disappearance of the peak at 310 nm and 480 nm, as monitored by UV-Vis (FIGS. 23B and 30A-30D). Moreover, RP-HPLC traces suggested higher monomer consumption after 2 h at 35° C. (FIGS. 23C and 31A-31B and Table 2). At five-fold higher concentration of 420 nM (50 U/mL) we observed almost complete monomer consumption (FIG. 23D).

TABLE 2
DA, TA and L-TY monomer consumption (%) after 2 h
reaction as determined via RP-HPLC.

| | Temperature (° C.) | |
|---|---|---|
| Monomer | rt | 35° C. |
| DA | 65% | 91% |
| TA | 76% | 86% |
| L-Tyr | 55%* | 86% |

*After 2 h reaction, great portion of the consumed monomer was not polymerized into melanin, but still present in the reaction mixture as oxidized species, which were detected by HPLC (FIGS. 23A-23D).

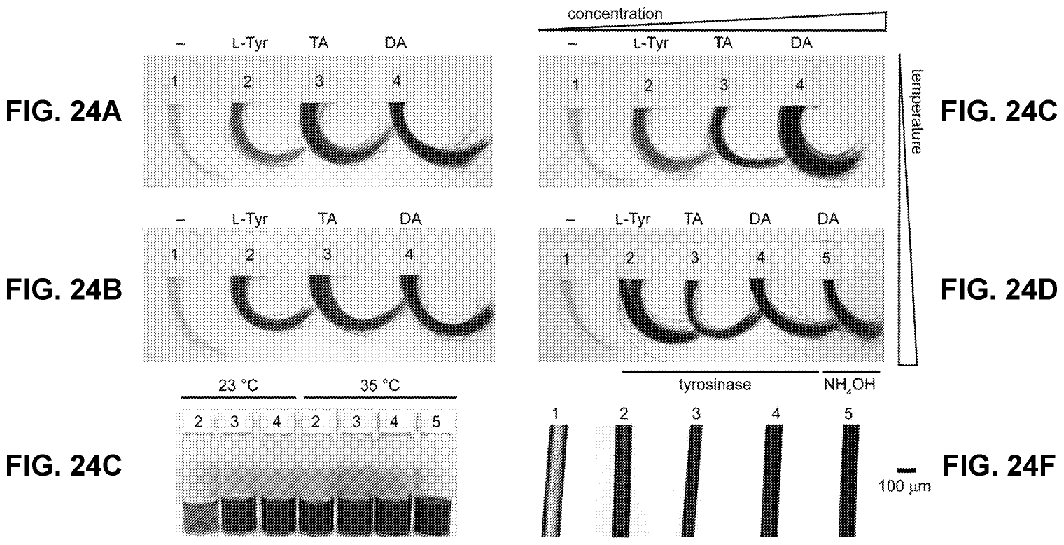

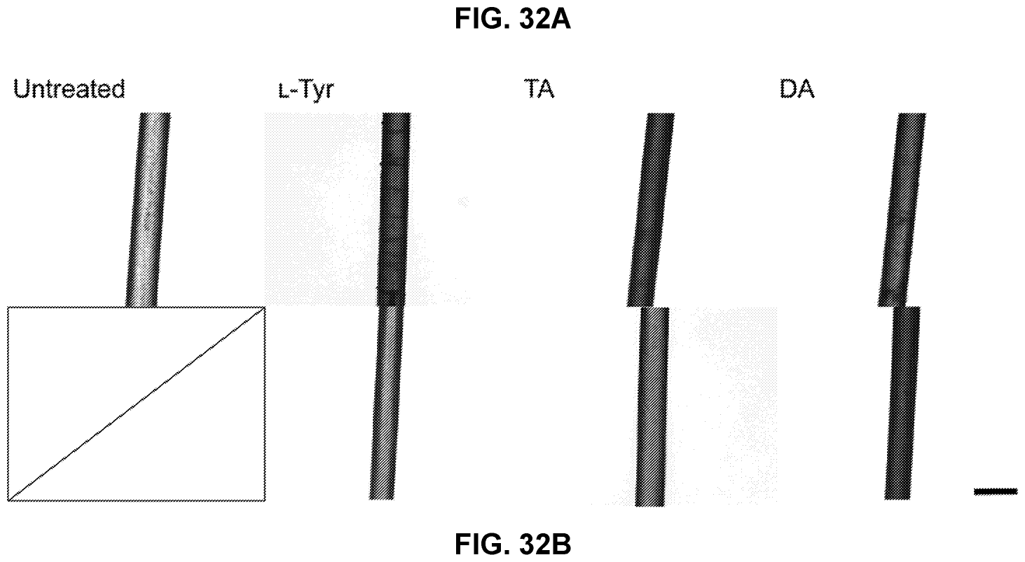

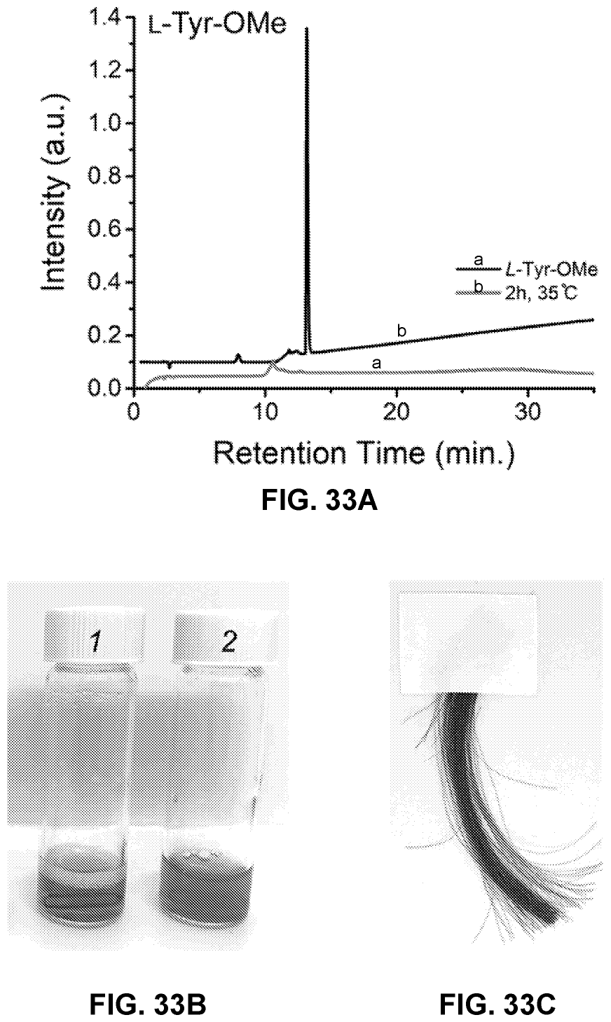

Similar reactivity was also obtained in the presence of blond hair (FIGS. 24A-24F). While initial screenings performed at rt using 2.6 mM to 3.6 mM monomers yielded dark blond/light brown hair colors (FIG. 24A), optimized enzymatic reactions (35° C., pH 7) produced a more convincing color transition (FIG. 24B). Higher concentration of both monomer and enzyme (36 mM TA or 26 mM DA and 840 nM tyrosinase) also enhanced darkening (FIG. 24C) and a combination of the optimized parameters (35° C., pH 7) and concentrations resulted in the darkest hair colors (FIG. 24D), which are comparable to those achieved via basic oxidation of dopamine in 3% aq. NH₄OH (FIG. 24F and FIGS. 32A-32B). However, it should be noted that L-Tyr could not be directly compared to the other monomers, as it could only be used at <5 mM due to limited solubility. To enhance solubility and to compare the performance of the different monomers at equal concentrations, we repeated the same reaction using L-tyrosine methyl ester (L-Tyr-OMe), which readily dissolves in water at 26 mM (5 mg/mL). This monomer is a good substrate for mushroom tyrosinase (FIGS. 33A-33C). However, despite successful hair darkening, the colors of both the reaction solution and the resulting coated hair were quite different from those generated using the other monomers investigated in this study (FIG. 24D and FIG. 24E). Hence, we limited this study to 2.8 mM (0.5 mg/mL) L-Tyr. To assure its complete conversion and to maximize hair darkening, we employed higher tyrosinase concentrations at 420 nM (sample 2 in FIG. 24D). For future applications, the development of more sophisticated formulations might enhance L-Tyr solubility and allow for higher reaction concentrations.

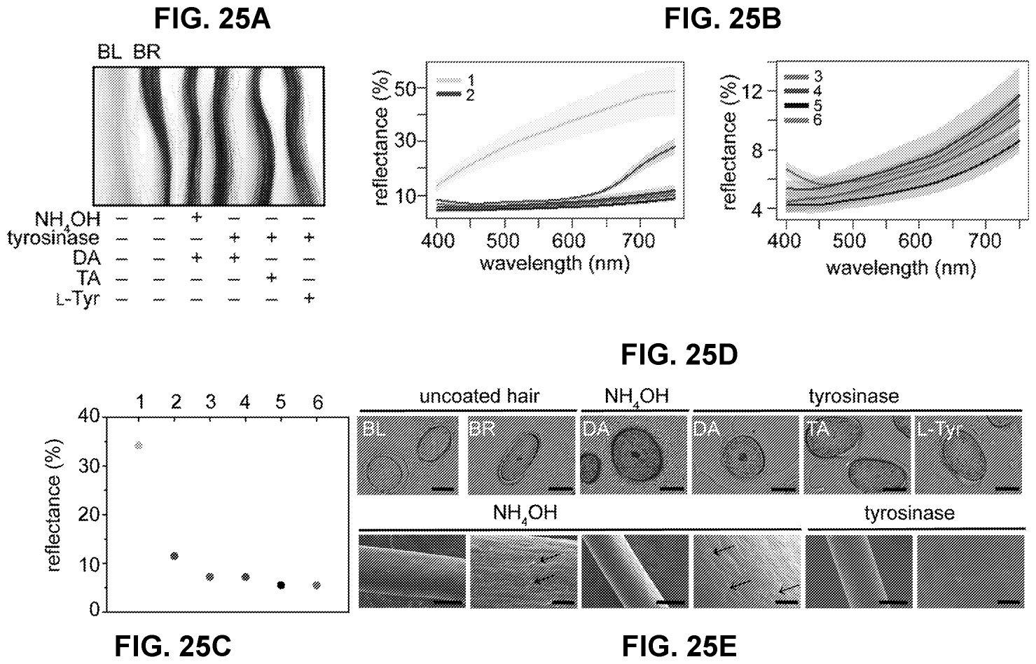

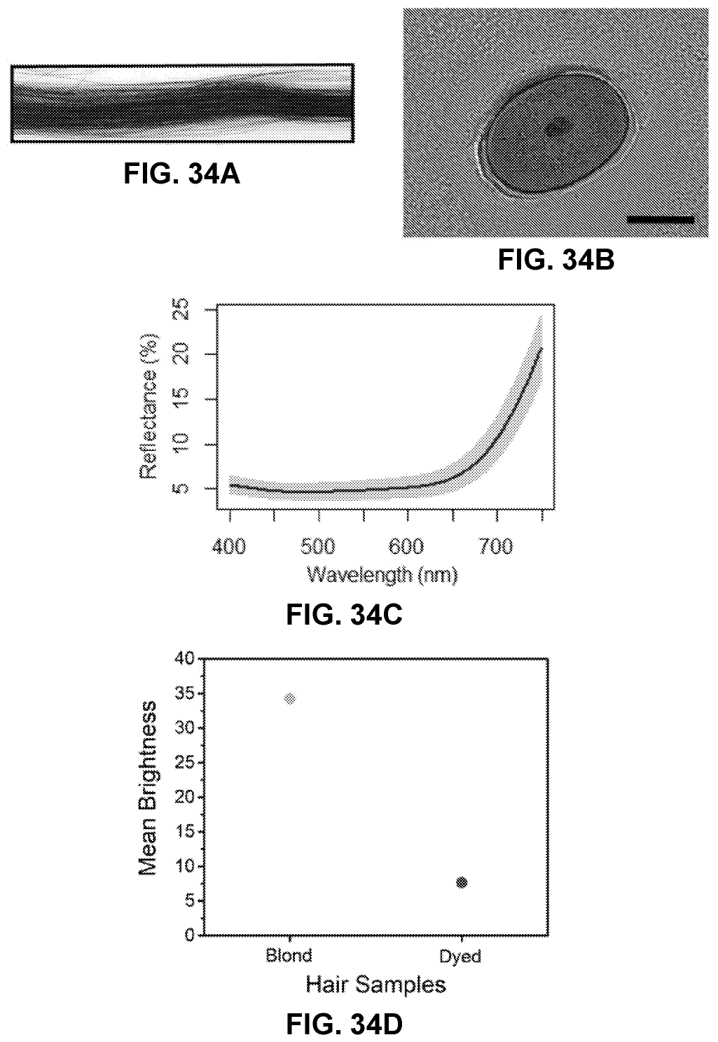

To gain information about the dyeing mechanism of the optimized chemoenzymatic reaction, we more closely investigated hair optical properties and color distribution (FIGS. 25A-25E). Hair photographs (FIG. 25A), as well as optical reflectance measurements (FIG. 25B) for the stained samples showed comparable outer hair darkening across the different tested coating conditions (basic oxidative polymerization of DA and chemoenzymatic oxidative polymerization of DA, TA and L-Tyr, samples 3-6, FIGS. 25A-25B). All coated samples were characterized by slightly lower reflectance at higher wavelengths (red region) as compared to natural brown hair, which color appeared warmer (sample 2 in FIGS. 25A-25B). Among coated samples, hair dyed via dopamine oxidation (samples 3-4 in FIGS. 25A-25B) more closely resembled natural brown hair. The brightness of all dark hair samples (samples 2-6) was also comparable and, expectedly, much lower compared to the uncoated, blond hair (FIG. 25C). Inner color distribution, determined via optical microscopy analysis of hair cross-sections (FIG. 25D), highlighted a slightly different dyeing mechanism. While in the case of chemoenzymatic reactions, the color remained localized on the outer layer (FIG. 25D), basic oxidative polymerization of DA resulted in coloration of both surface and inner hair cortex. Such diffuse inner hair color derives from the use of base, which is known to create hair cuticle swelling and, in turn, causes deeper penetration into the hair cortex. This is the primary dyeing process used in conventional hair dyes (FIGS. 34A-34D). Here, instantaneous dopamine oxidation in the presence of 3% aq. NH₄OH leads to rapid polymerization, with a majority of the material localized on the hair surface, and a smaller percentage penetrating into the hair cortex. In contrast, the lack of base in the chemoenzymatic reaction causes extremely low levels of penetration into the hair cortex (samples 4-6, FIG. 25D), therefore providing efficient and comparable hair color with minimum perturbation to the inner cortex.

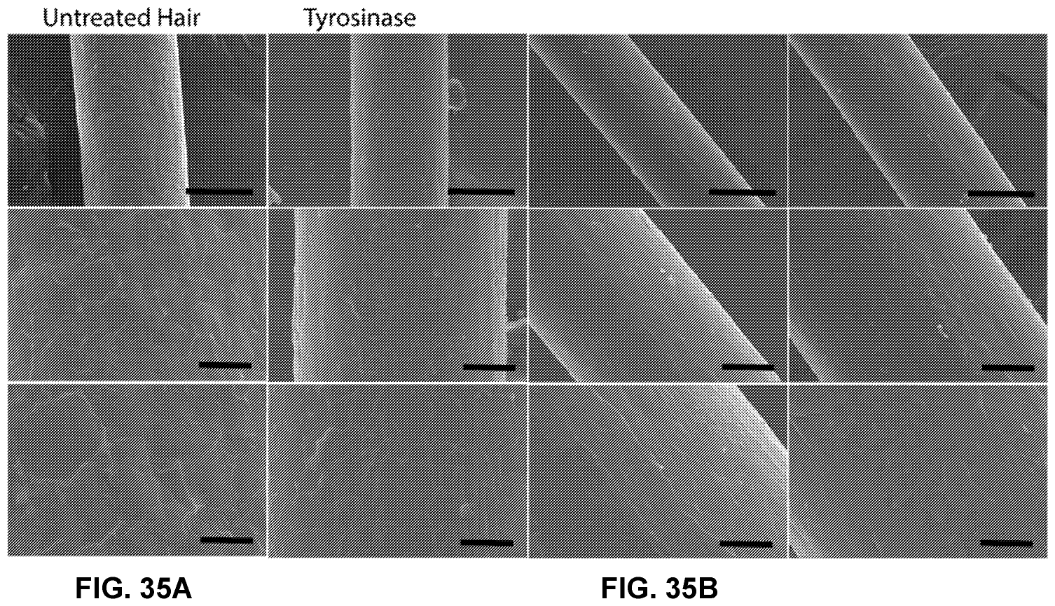

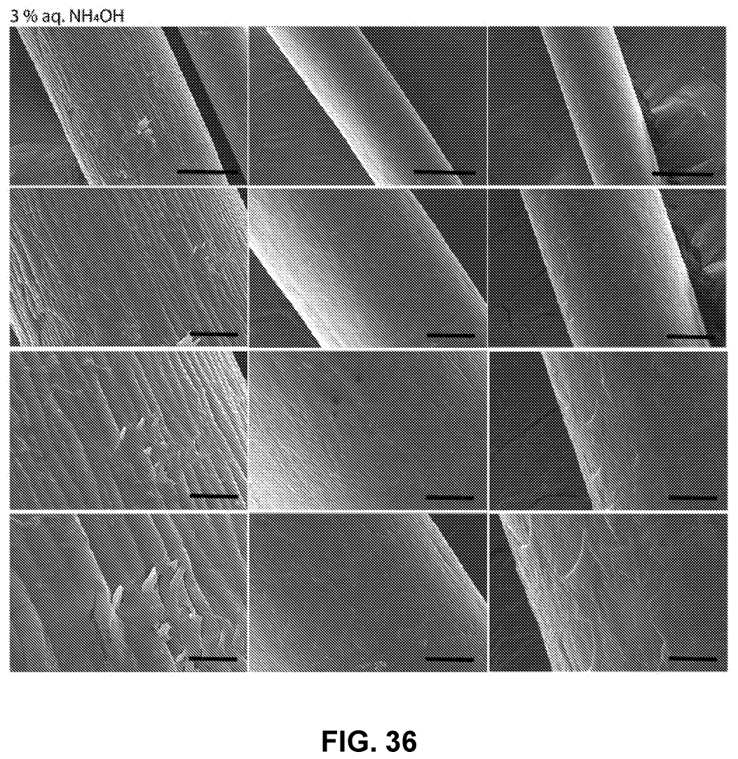

Apart from preventing penetration into the hair cortex and minimizing inner hair perturbation, enzymatic reactions also limit damage to the outer hair layers as compared to basic oxidative protocols. It is known that cuticle swelling in basic conditions can lead to potential hair damage or morphological changes of the hair surface.^{32,33} Indeed, commercial dyes are designed to do this as part of the process of entraining dyes into hair fibers. To demonstrate how chemoenzymatic dyeing contrasts with this process, hair samples were treated using either basic or enzymatic additives in the absence of monomers, allowing us to investigate the outer hair morphology in the absence of any coating being formed. While chemoenzymatic reactions did not result in any visible hair damage, as expected, basic oxidation led to a more evident lifting of outer cuticles (FIG. 25E and FIGS. 35A-35B and 36).³² While both basic and chemoenzymatic oxidation of DA and precursors/derivatives are viable synthetic paths for mimicking the natural pigment melanin, the chemoenzymatic approach is far milder, with the potential for minimization of skin irritation and prevention of hair damage.

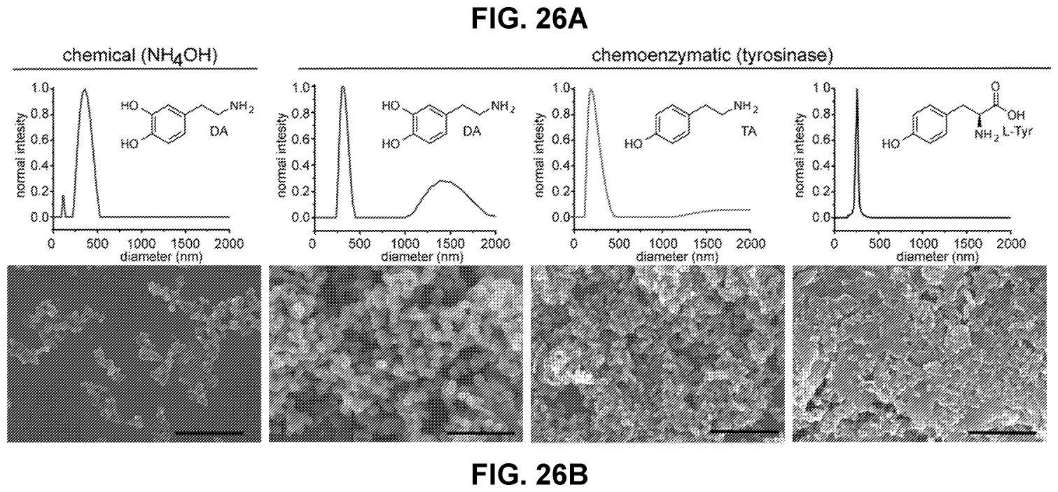

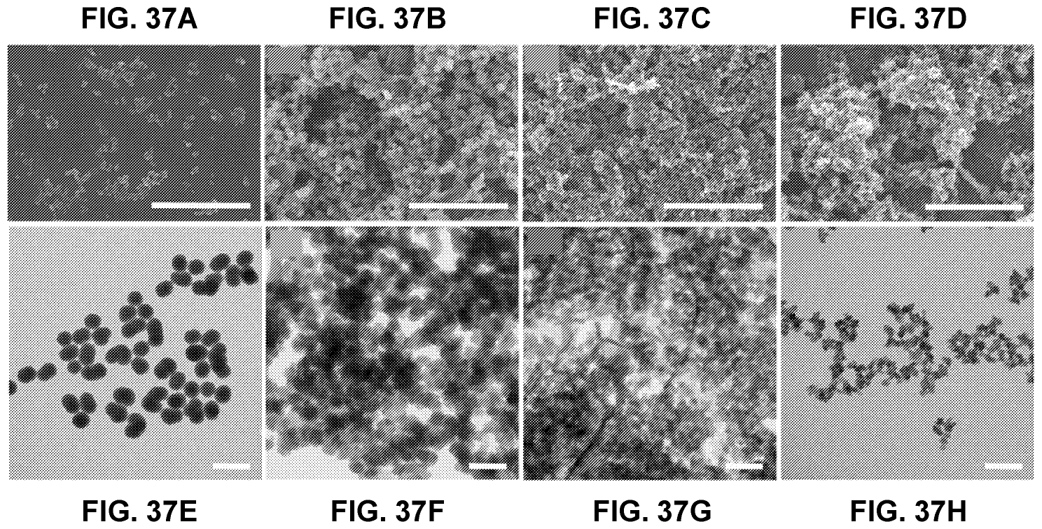

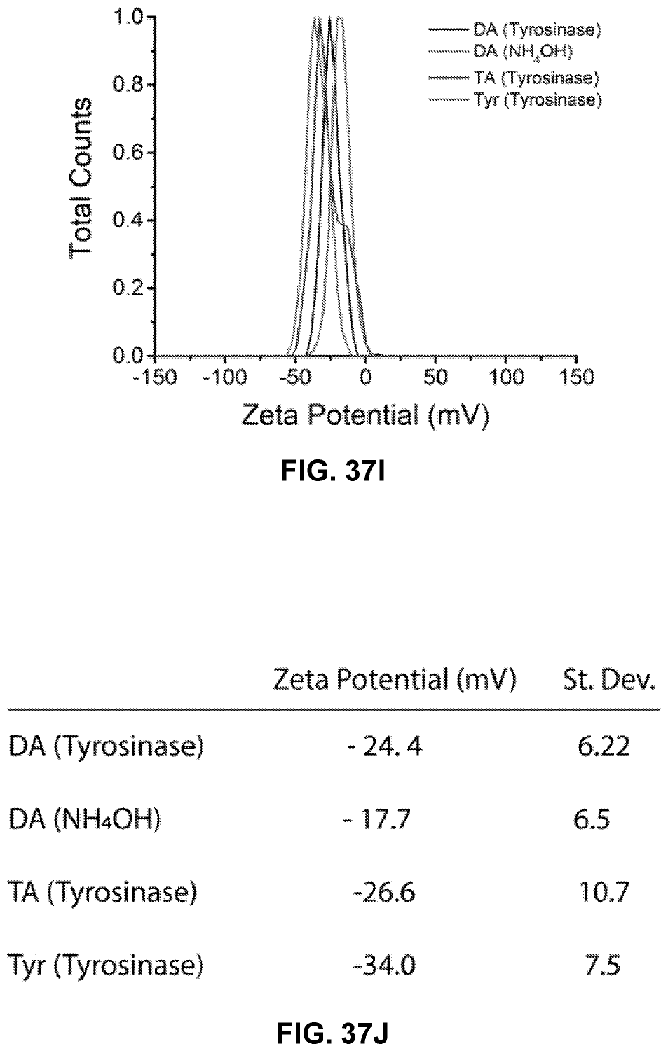

Next, we investigated the deposition mechanism and hair surface morphology. Analysis of solutions derived from the dyeing reaction (35° C., pH 7) (FIGS. 26A-26B) suggested that oxidative chemoenzymatic polymerization of all monomers produced nanoparticles (in the size range 200-500 nm) comparable to those obtained via basic oxidation of dopamine as determined by DLS (FIG. 26A). All nanoparticles had a round appearance as determined by both SEM (FIG. 26B and FIGS. 37A-37J) and TEM (FIGS. 37A-37J) imaging and a negative zeta potential (FIGS. 37A-37J).

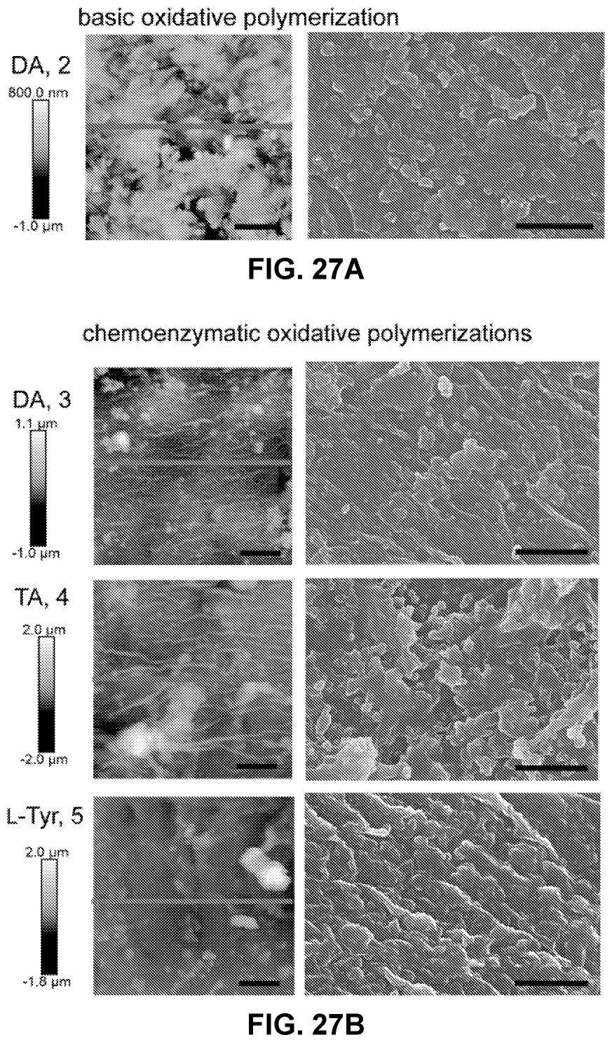

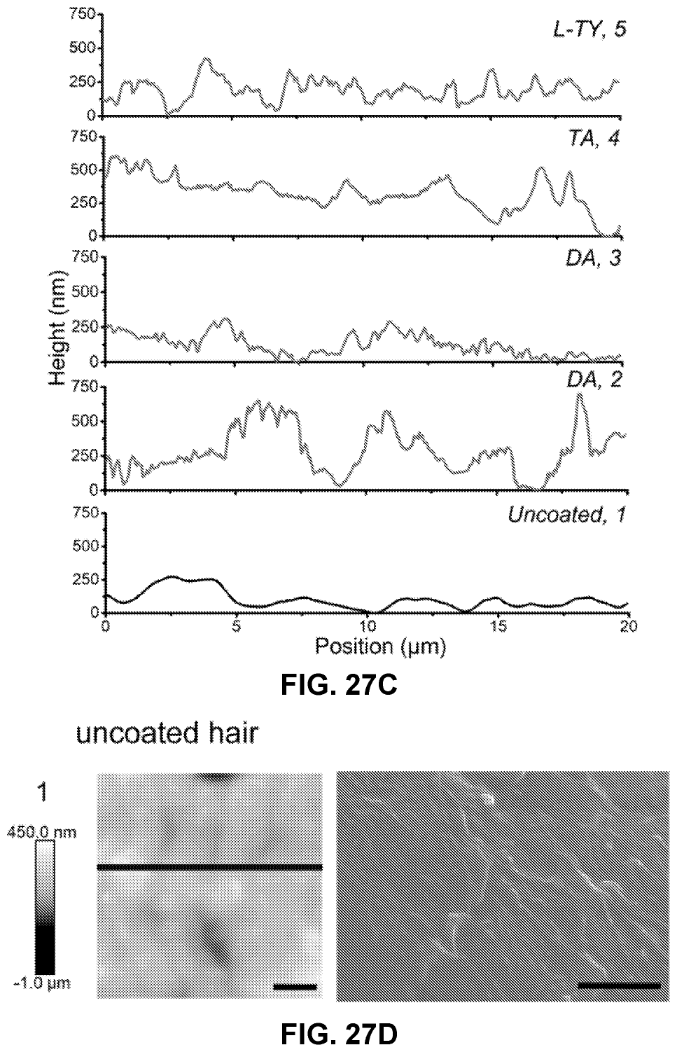

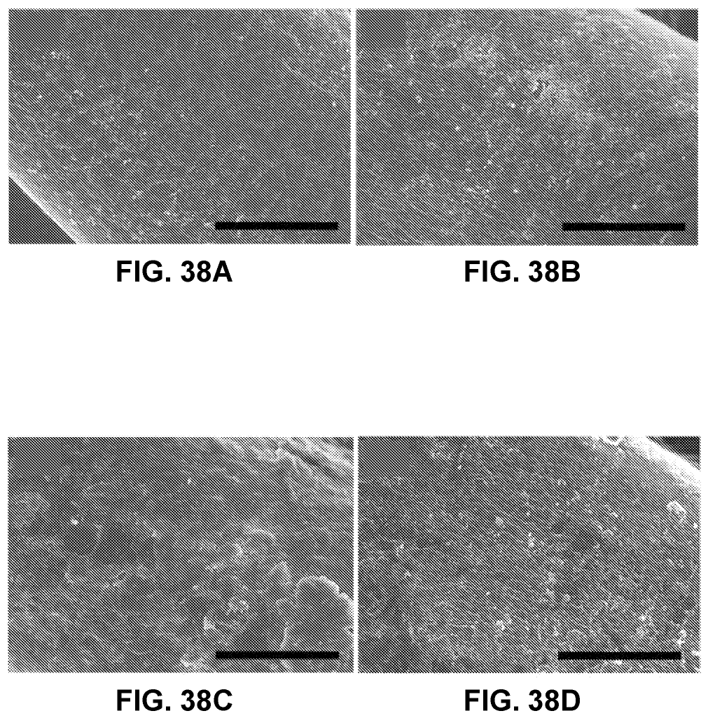

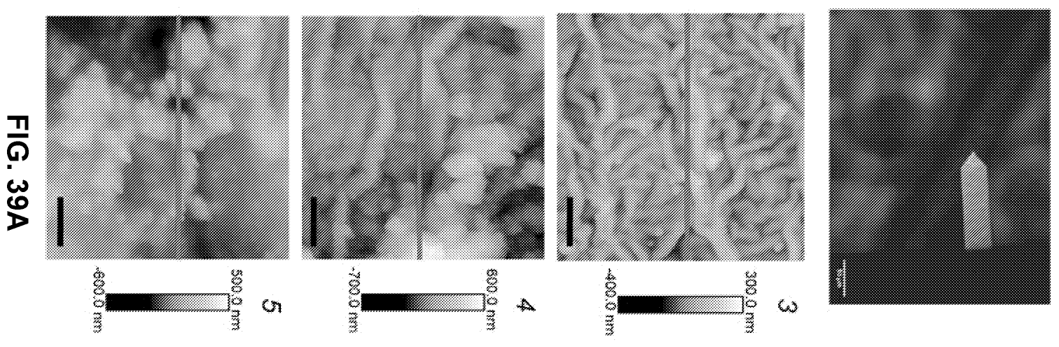

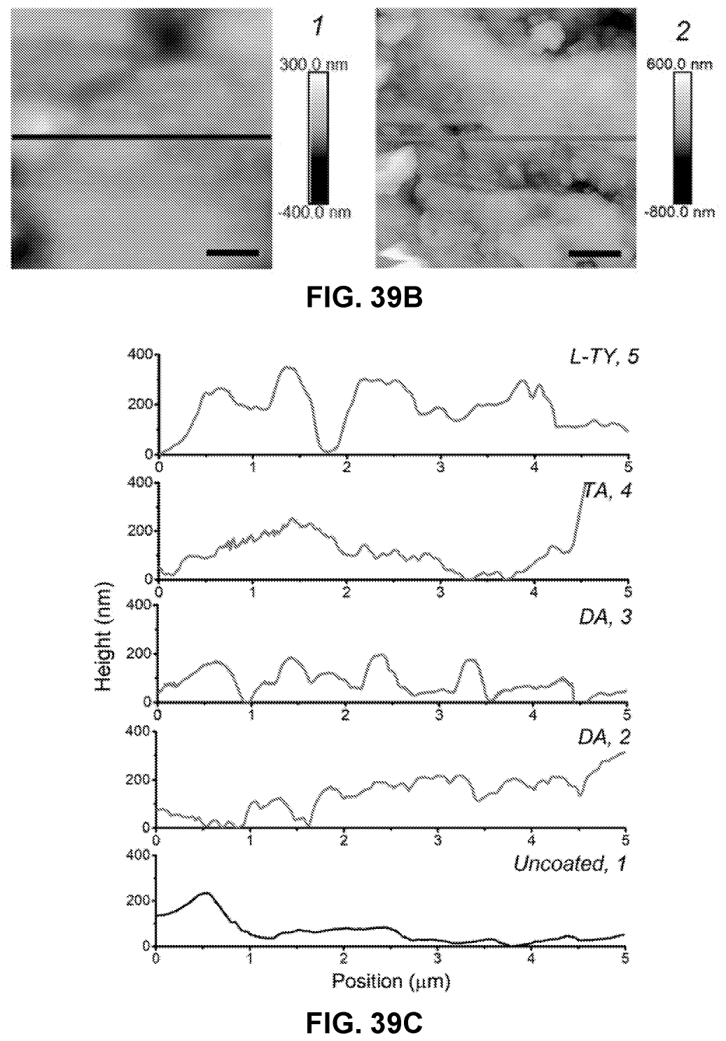

However, SEM and AFM imaging of the hair fibers coated using this protocol revealed slightly different phenomena at the hair surface (FIGS. 27A-27D and FIGS. 38A-38D and 39A-39C). While basic oxidative polymerization of dopamine resulted in nanoparticle deposition (FIG. 27A), chemoenzymatic reactions led to both nanoparticles as well as film deposition on hair surface, as indicated by the presence of larger and in some cases elongated deposits (FIG. 27B). Analysis by AFM imaging confirms rougher fiber profiles (FIG. 27C) than uncoated, smoother hair (FIG. 27D). In addition, AFM confirmed that coatings generated through chemoenzymatic reactions contain both nanoparticles and more elongated, flatter depositions, which extend across the hair surface.

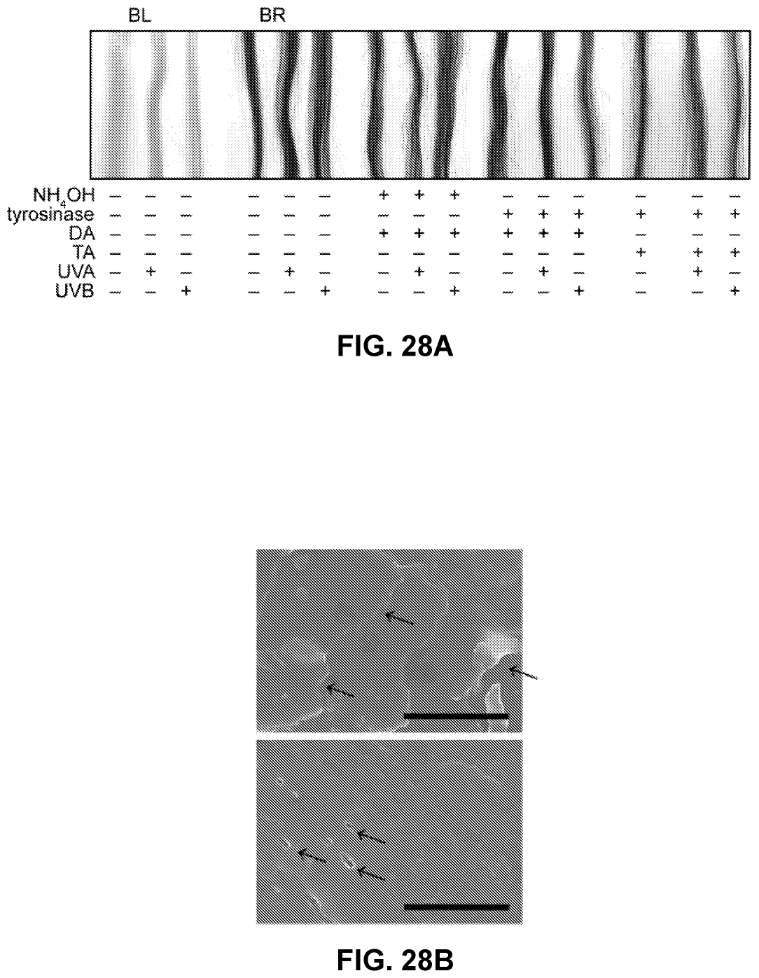

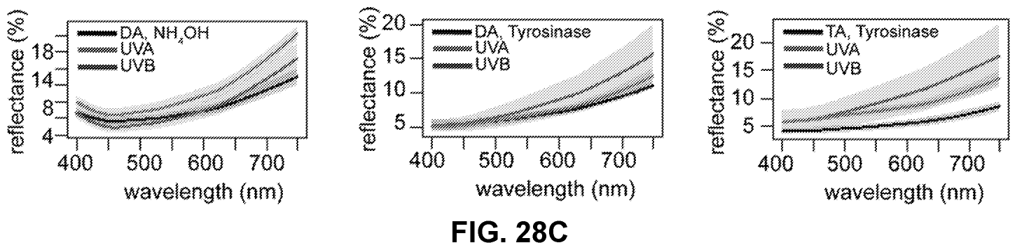

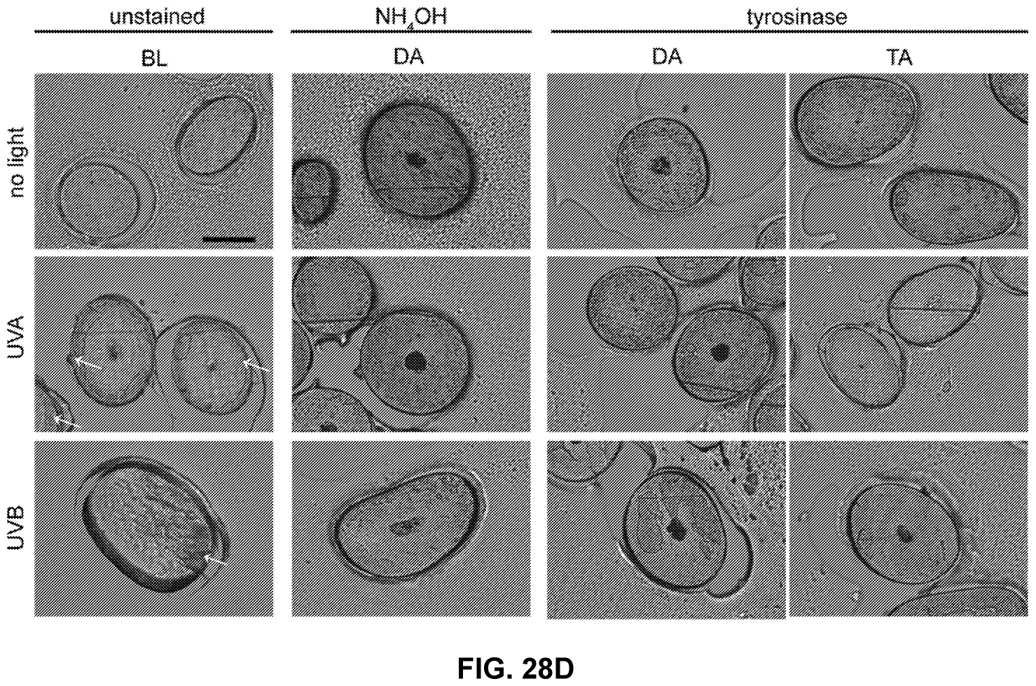

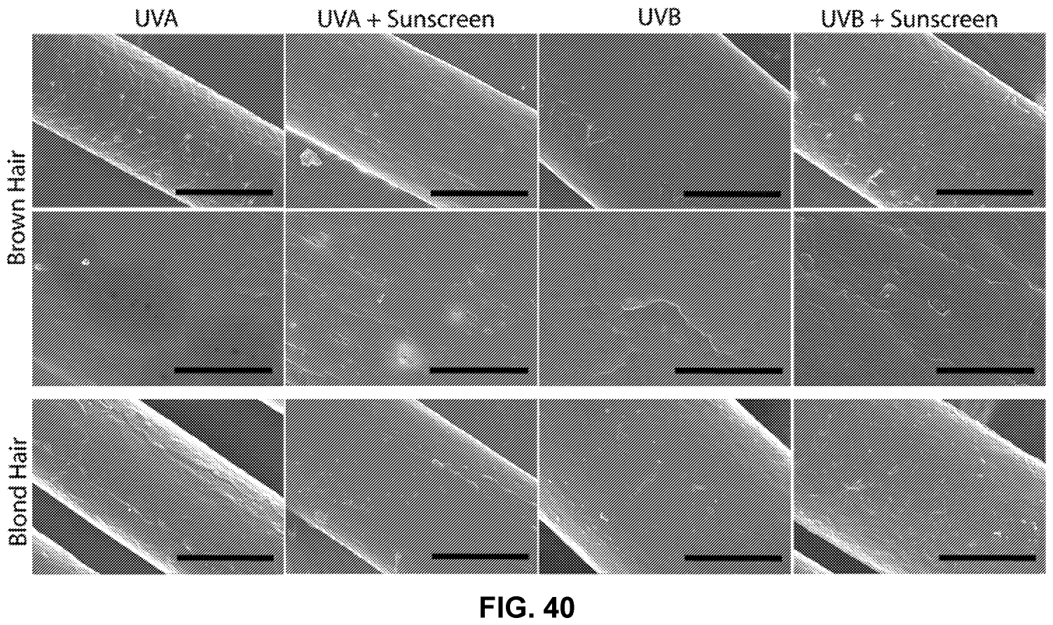

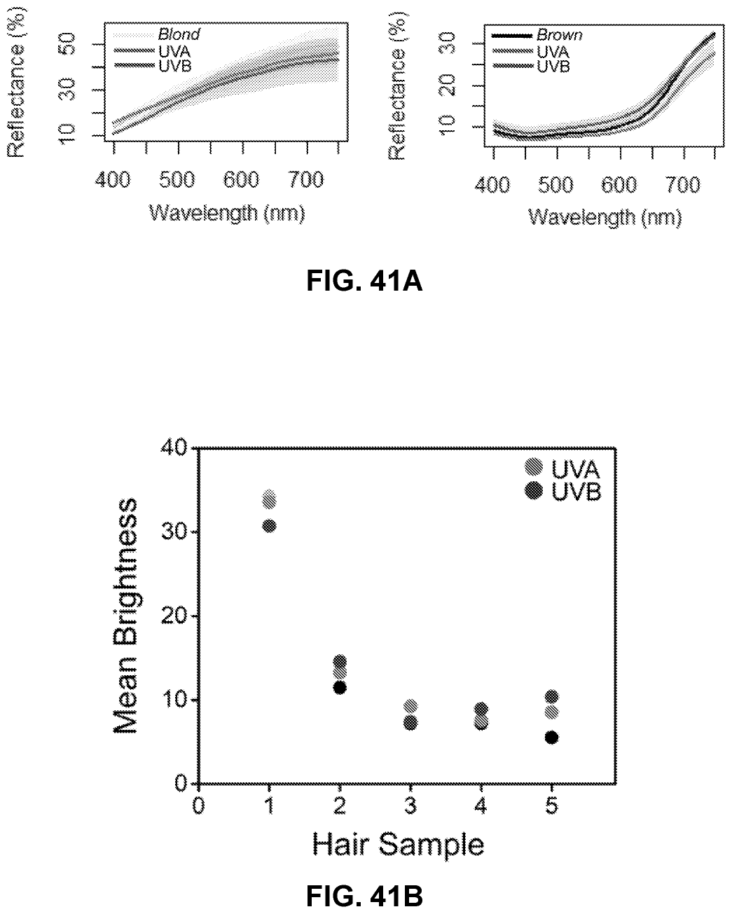

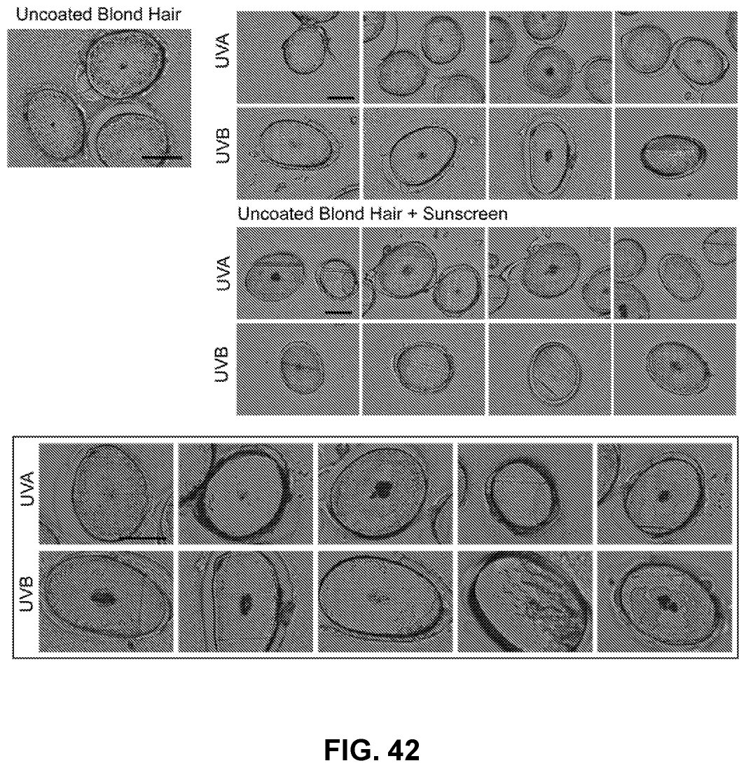

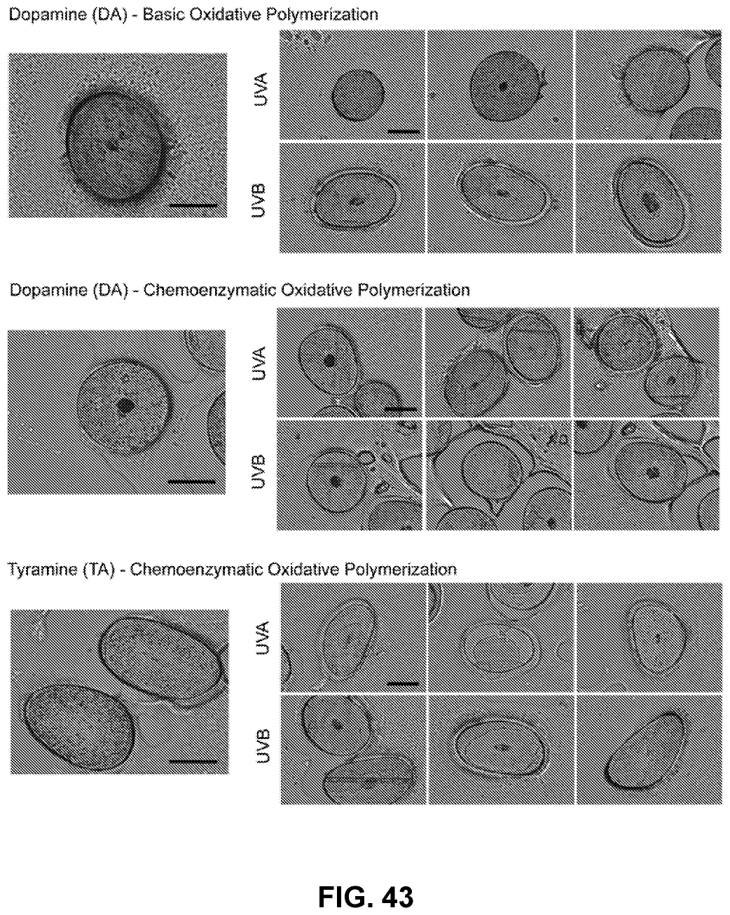

With the next set of experiments, we aimed to investigate if such brown/black coatings mimicked the natural properties of melanin. In nature, melanin not only provides color to the hair and skin, but can provide photochemical protection from solar radiation. In hair, melanin absorbs a large fraction of incident radiation and also captures many of the free radicals generated from UV radiation absorption by amino acids, preventing the transport of these free radicals into the keratin matrix.^{34-37,43} The extent of hair damage depends upon the nature of the hair pigments, with dark hair being more photostable than blond hair.⁴³⁻⁴⁵ This derives from the protective action of the melanin-rich cortex in black hair, which shows only a slight modification of fiber proteins under irradiation.⁴⁵ However, since the majority of melanin pigments in hair are located in the inner cortex, and since penetration into the hair layers reduces radiation intensity, damage can occur only after prolonged exposure, and most damage occurs primarily in the outer cuticle.^{43,46-49} Owing to the intense UV absorption of hair coated with PDA,¹⁹ we reasoned that synthetic melanin deposition on hair could mitigate photodamage by shielding the covered outer hair layers from UV radiation. To date, the majority of reported biochemical and photochemical changes caused in hair by radiation exposure focuses on evaluation of physical changes to the outer components of hair anatomy.^{43,46,49} As a complement, we investigated the effects of developed melanin coatings on morphological and optical changes of outer hair layers upon UV irradiation. As shown in FIGS. 28A-28D, we exposed untreated hair, hair coated with synthetic melanin via basic oxidative polymerization of DA, and hair treated with chemoenzymatic oxidative polymerization of DA or TA, to either UVA (315-400 nm) or UVB (280-315 nm) for 10 days (see Experimental Section for lamp specifications) and analyzed the morphological changes of the outer layers (FIGS. 28A-28D). In this particular study we omitted samples prepared using the monomer L-Tyr, since the working concentration was different from that of the other monomers. Photographs of hair samples before and after UV irradiation are shown in FIG. 28A. SEM analysis clearly demonstrated that this exposure time is sufficient to damage the hair (FIG. 286).⁴⁶⁻⁵⁰ Damage upon both UVA and UVB irradiation was visible in the outer layer of both blond and naturally brown hair samples (FIG. 28B and FIG. 40). This effect was visible even in the case of hair treated with a commercial broad-spectrum hair sunscreen prior to irradiation (FIG. 40), indicating that repetitive application of sunscreen is a fundamental requirement for efficient photoprotection. We note that synthetic melanin-treated hair could not be screened for such morphological changes because the coating covers the hair surface. Reflectance spectra of coated hair samples before and after irradiation (FIG. 28C and FIGS. 41A-41B) revealed minor increases in reflectance values. The largest change occurred when TA was employed as a monomer, and in particular upon UVB irradiation. However, the prolonged irradiation employed in this study and color alterations comparable to those observed for uncoated hair samples (FIGS. 41A-41B) suggest that the developed coatings can resist prolonged irradiation without complete fading and unmasking of the blond hair substrates. To demonstrate that such melanin deposits have the potential to shield the inner layers from the effects of irradiation, we analyzed hair cross-sections by optical microscopy (FIG. 28D and FIGS. 42 and 43). While untreated hair showed the highest levels of cuticle lifting, exfoliation, and damage, even when pre-treated with sunscreen, the synthetic melanin coated hair showed intact hair sections, independent of the deposition method (basic versus chemoenzymatic oxidations). Together, these results indicate that synthetic melanin coatings deposited on hair surface not only impart natural looking color to hair fibers, but also have the potential to interact with solar radiation, protecting the inner hair layers from photodamage.

With increasing concerns regarding the potential toxicity of commercial hair dye formulations,^{4,12} bioinspired synthetic approaches become increasingly attractive alternatives.²⁰ In this work, we demonstrated that a protective and pigmented layer can be achieved in hair coating via mild enzymatically induced reactions. Previously, hair coloration could be obtained using basic conditions and oxygen from air to drive dopamine oxidative polymerization to yield a PDA coating. By contrast, the enzymatically driven approach occurs at neutral pH and allows the use of a variety of monomers such as L-Tyr, TA, DA and derivatives. Moreover, morphological and optical analyses of hair exposed to UV radiation before and after base- or chemoenzymatic-induced deposition of synthetic melanin suggest that these coatings have the potential to absorb radiation, thereby protecting the inner fibers from photodamage. Overall, this work describes chemoenzymatic synthetic protocols toward artificial melanin depositions and provides a fast, mild, and efficient route to multifunctional bio-inspired hair pigmentation and fiber coatings.

Materials and Methods. Mushroom tyrosinase, 28-30% (w/v) aq. NH₄OH and L-Tyr were purchased from Sigma Aldrich. Dopamine hydrochloride (DA HCl) was obtained from Frontier Scientific, and tyramine (TA) was purchased from Chem-Impex. Blond (level approximately 9/10) human hair samples were purchased from Jerome Krause Fashion Hair (Evanston, Ill.). UV-Vis spectroscopy kinetics were performed with an Agilent Cary 100 UV-Vis spectrometer using quartz cuvettes. RP-HPLC analyses of monomer consumptions were performed on a Jupiter 4μ Proteo 90A Phenomenex column (150×4.60 mm) with a binary gradient, using a HitachiElite LaChrom 2130 pump that was equipped with a Hitachi-Elite LaChrom L-2420 UV-Vis detector. Scanning electron microscopy (SEM) images were acquired on an FEI Quanta 650 SEM and a Hitachi SU8030 SEM. Scanning transmission electron microscopy (STEM) images were obtained on a Hitachi HD2300 STEM operating at 200 kV. Hair samples were imaged using a Leica BM6B wide field optical microscope. AFM was performed using a Bruker Icon AFM and optical reflectance measurements in the visible spectrum were acquired using an AvaSpec-ULS2048L StarLine versatile fiber-optic spectrometer and the AvaSpec-NIR256/512-2.0/2.2TEC NIRLine Near-Infrared fiber-optic spectrometer in combination with an AvaLight-HAL-(S)-MINI Tungsten-Halogen Light Source+AvaLight-DH-S Deuterium-Halogen Light Source.

UV-Vis Spectroscopy of PDA Solutions. Initially, reactions were performed in both water and DPBS to demonstrate that both vehicles can be used. Water was then used for the following studies. The pH was adjusted to 7.4 after preparing 2.8 mM L-Tyr, 3.6 mM TA or 2.6 mM DA (0.5 mg/mL) in water. Solutions were then stirred either at rt or at 35° C. for 6 h. UV-Vis time-dependent spectra were recorded by withdrawing 10 μL solution from each sample at different time intervals. The withdrawn samples were diluted to 1 mL (either water or DPBS) and analyzed. For all experiments 84 μM tyrosinase (10 U/mL) was used.⁴⁰

Monitoring Monomer Consumption via RP-HPLC studies. Monomer consumption after a 2 h reaction was determined by RP-HPLC. Reactions were performed either at rt or at 35° C. and using either 84 nM or 840 nM (five fold) tyrosinase. The HPLC solvent system consisted of (A) 0.1% TFA in water and (B) 0.1% TFA in acetonitrile, with a gradient of 2% B to 65% B over 30 minutes.

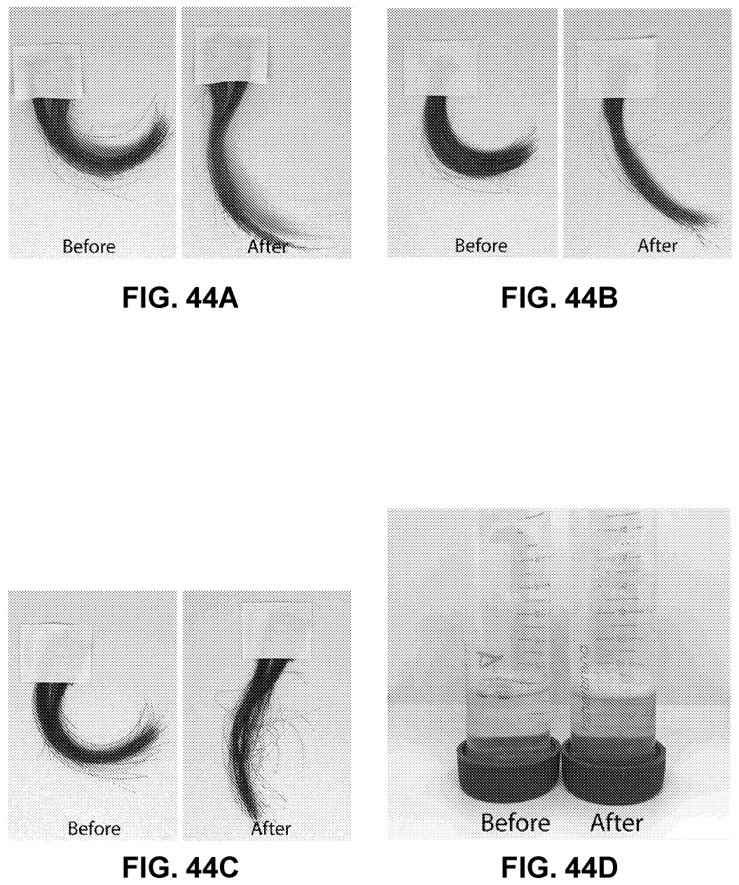

Hair Dyeing. Hair dyeing was initially carried out using either 2.8 mM L-Tyr, 3.6 mM TA or 2.6 mM DA (0.5 mg/mL) and 84 nM tyrosinase. The volume of the solutions was selected based on the size of hair samples (generally 4-5 mL for hair samples approximately 3 cm long). After 2 h reaction at either rt or 35° C., hair samples were washed 5 times with distilled water as previously reported.²⁰ In a proof-of-concept experiment, color persistence was assessed for hair dyed via chemoenzymatic polymerizations. Washes were performed by vortexing the hair for 1 minute in a 10% shampoo solution. This process was repeated 10 times (FIGS. 44A-44D). To achieve maximum hair darkening, the concentration of both substrate and enzyme were increased 10 fold (36 mM TA or 26 mM DA (5 mg/mL) and 840 nM (100 U/mL) tyrosinase). In the case of L-Tyr, the monomer concentration was limited to 2.8 mM (0.5 mg/mL) due to its lower solubility in water. In this case, 420 nM (50 U/mL) tyrosinase was used to assure complete L-Tyr monomer oxidation and therefore maximize hair darkening. As a comparison, hair was dyed via basic oxidative polymerization using 26 mM DA and 3% aq. NH₄OH solutions as previously described.²⁰ Such optimized oxidations were performed for 2 h at 35° C.

Characterization of Nanoparticles in the Reaction Solutions. After a 2 h reaction, the solutions obtained using the above reported conditions were centrifuged twice, diluted in water, and analyzed by Dynamic Light Scattering (DLS). The remaining part was drop casted and evaporated onto a silicon wafer substrate at rt and coated with 6 nm osmium and imaged using a Hitachi SU8030 cFEG SEM at an accelerating voltage of 10 kV and an emission current of 15 μA.

SEM of Hair Samples. Hair samples were adhered onto aluminum SEM stubs by pressing lightly onto carbon tape using a clean glove. The samples were coated with 10 nm of osmium and imaged using a Hitachi SU8030 cFEG SEM at 10.0 kV and 15 μA or an FEI Quanta 650 at 15 kV.

AFM of Hair Samples. Samples held at one end by tweezers were laid on carbon sticky film on a metal disc. 8 mm sections were cut from the main hair to remain on the film. This was sufficiently stable for imaging in tapping mode in air with RTesp150 tips with a spring constant of 5 N/m and nominal tip radius of 8 nm on a Bruker Icon AFM.

Preparation of Hair Cross-Sections for Optical Microscopy. Hair was embedded with optimal cutting temperature (OCT) medium and 10 μm sections were cut at −20° C. and deposited onto glass microscope slides.

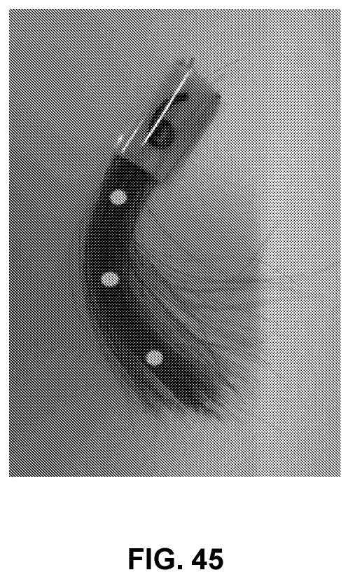

Optical Reflectance Spectroscopy. Specular reflectance measurements of untreated, UVA, and UVB treated hair samples in the visible spectrum were measured using a probe holder fitted and held at a 90° angle on top of the sample. Three reflectance measurements were taken per hair sample (FIG. 45). Analysis of the spectral data and calculation of mean brightness were performed in R (R Core Team (2018). R: A language and environment for statistical computing. R Foundation for Statistical Computing, Vienna, Austria. https://www.R-project.org/) using the pavo⁵¹ package.

UVA and UVB Irradiation. Hair samples were exposed to either UVA or UVB for 10 days. UVA irradiation was performed using a USpicy USND-3601 Professional UV GEL Lamp, with 36 W (4×9 W bulbs) source. UVB irradiation was performed using a Gardco UV-X-15B, equipped with 312 nm, 15 W lamp. The irradiance at the sample surface was found to be 3.8±0.27 mW/cm² in the case of UVA and 2.1±0.15 mW/cm² for UVB as measured with a Thorlabs S120VC photodiode attached to a PM100D console left in-situ for one hour to reach thermal equilibration.

### REFERENCES ASSOCIATED WITH EXAMPLE 3

1. Cordero, R. J. B.; Casadevall, A., Melanin. Curr Biol 2020, 30 (4), R142-R143.
2. Riley, P. A., Melanin. Int J Biochem Cell B 1997, 29 (11), 1235-1239.
3. Nishimura, E. K.; Granter, S. R.; Fisher, D. E., Mechanisms of hair graying: Incomplete melanocyte stem cell maintenance in the niche. Science 2005, 307 (5710), 720-724.
4. Eberle, C. E.; Sandler, D. P.; Taylor, K. W.; White, A. J., Hair dye and chemical straightener use and breast cancer risk in a large US population of black and white women. International Journal of Cancer 2019, 147, 383-391.
5. Coenraads, P. J.; Aberer, W.; Cristaudo, A.; Diepgen, T.; Holden, C.; Koch, L.; Schuttelaar, M. L.; Weisshaar, E.; Fuchs, A.; Schlotmann, K.; Goebel, C.; Blomeke, B.; Krasteva, M., The Allergy Alert Test: Introduction of a Protocol Suitable to Provide an Alert Signal in p-Phenylenediamine-Allergic Hair Dye Users. Dermatitis 2018, 29(5), 231-232.
6. Han, J. H.; Lee, H. J.; Bang, C. H.; Lee, J. H.; Park, Y. M.; Lee, J. Y., P-Phenylenediamine Hair Dye Allergy and Its Clinical Characteristics. Ann Dermatol 2018, 30 (3), 316-321.
7. Hink, E.; de Winter, J. P., Hair-dye allergy: a coloured case. Eur J Pediatr 2006, 165 (3), 195-6.
8. Martyn-Simmons, C. L.; Holden, C. A., Adult T-cell leukaemia/lymphoma masquerading as a hair dye allergy. Br J Dermatol 2006, 154 (1), 196-7.
9. Sosted, H.; Menne, T., Allergy to 3-nitro-p-hydroxyethylaminophenol and 4-amino-3-nitrophenol in a hair dye. Contact Dermatitis 2005, 52 (6), 317-9.
10. Ferguson, F. J.; Pongpairoj, K.; Basketter, D. A.; White, I. R.; McFadden, J. P., Addressing the conundrums of p-phenylenediamine hair dye allergy by applying Friedmann's principles of contact sensitization. Contact Dermatitis 2019, 80 (4), 234-237.
11. Kim, K. H.; Kabir, E.; Jahan, S. A., The use of personal hair dye and its implications for human health. Environ Int 2016, 89-90, 222-227.
12. Alipour, S., Comments on: Hair dye and chemical straightener use and breast cancer risk in a large US population of black and white women. International Journal of Cancer 2020, 146 (9), 2651-2651.
13. Au, K. M.; Lu, Z. H.; Matcher, S. J.; Armes, S. P., Polypyrrole Nanoparticles: A Potential Optical Coherence Tomography Contrast Agent for Cancer Imaging. Adv Mater 2011, 23 (48), 5792-+.
14. Guo, X. J.; Cao, B.; Wang, C. Y.; Lu, S. Y.; Hu, X. L., In vivo photothermal inhibition of methicillin-resistant Staphylococcus aureus infection by in situ templated formulation of pathogen-targeting phototheranostics. Nanoscale 2020, 12 (14), 7651-7659.
15. Jeon, J. R.; Le, T. T.; Chang, Y. S., Dihydroxynaphthalene-based mimicry of fungal melanogenesis for multifunctional coatings. Microb Biotechnol 2016, 9 (3), 305-15.
16. Yang, J.; Zhai, S.; Qin, H.; Yan, H.; Xing, D.; Hu, X., NIR-controlled morphology transformation and pulsatile drug delivery based on multifunctional phototheranostic nanoparticles for photoacoustic imaging-guided photothermal-chemotherapy. Biomaterials 2018, 176, 1-12.
17. Im, K. M.; Kim, T. W.; Jeon, J. R., Metal-Chelation-Assisted Deposition of Polydopamine on Human Hair: A Ready-to-Use Eumelanin-Based Hair Dyeing Methodology. Acs Biomater Sci Eng 2017, 3 (4), 628-636.
18. Gao, Z. F.; Wang, X. Y.; Gao, J. B.; Xia, F., Rapid preparation of polydopamine coating as a multifunctional hair dye. Rsc Adv 2019, 9 (35), 20492-20496.
19. Dong, Y. Y.; Qiu, Y.; Gao, D.; Zhang, K. L.; Zhou, K.; Yin, H. G.; Yi, G. Y.; Li, J.; Xia, Z. N.; Fu, Q. F., Melanin-mimetic multicolor and low-toxicity hair dye. Rsc Adv 2019, 9 (58), 33617-33624.
20. Battistella, C.; McCallum, N. C.; Gnanasekaran, K.; Zhou, X.; Caponetti, V.; Montalti, M.; Gianneschi, N. C., Mimicking Natural Human Hair Pigmentation with Synthetic Melanin. ACS Central Science 2020, 6 (7), 1179-1188.
21. Guerra-Tapia, A.; Gonzalez-Guerra, E., Hair cosmetics: dyes. Actas Dermosifiliogr 2014, 105 (9), 833-9.
22. Prota, G., Melanins, melanogenesis and melanocytes: looking at their functional significance from the chemist's viewpoint. Pigment Cell Res 2000, 13 (4), 283-93.
23. D'Mello, S. A. N.; Finlay, G. J.; Baguley, B. C.; Askarian-Amiri, M. E., Signaling Pathways in Melanogenesis. Int J Mol Sci 2016, 17 (7).
24. Lai, X. L.; Wichers, H. J.; Soler-Lopez, M.; Dijkstra, B. W., Structure and Function of Human Tyrosinase and Tyrosinase-Related Proteins. Chem-Eur J 2018, 24 (1), 47-55.
25. Zaidi, K. U.; Ali, A. S.; Ali, S. A.; Naaz, I., Microbial tyrosinases: promising enzymes for pharmaceutical, food bioprocessing, and environmental industry. Biochem Res Int 2014, 2014, 854687.
26. Martinez, L. M.; Martinez, A.; Gosset, G., Production of Melanins With Recombinant Microorganisms. Front Bioeng Biotechnol 2019, 7, 285.
27. Ren, Q.; Henes, B.; Fairhead, M.; Thony-Meyer, L., High level production of tyrosinase in recombinant Escherichia coli. BMC Biotechnol 2013, 13, 18.
28. Seo, S. Y.; Sharma, V. K.; Sharma, N., Mushroom tyrosinase: Recent prospects. J Agr Food Chem 2003, 51 (10), 2837-2853.
29. Ba, S.; Kumar, V. V., Recent developments in the use of tyrosinase and laccase in environmental applications. Crit Rev Biotechnol 2017, 37 (7), 819-832.
30. de Faria, R. O.; Moure, V. R.; Amazonas, M. A. L. D.; Krieger, N.; Mitchell, D. A., The biotechnological potential of mushroom tyrosinases. Food Technol Biotech 2007, 45 (3), 287-294.
31. Panzella, L.; Ebato, A.; Napolitano, A.; Koike, K., The Late Stages of Melanogenesis: Exploring the Chemical Facets and the Application Opportunities. Int J Mol Sci 2018, 19 (6).
32. Ali, N.; Marsh, J.; Godfrey, S.; Williams, D. R., Aqueous MEA and Ammonia Sorption-Induced Damage in Keratin Fibers. ACS Omega 2018, 3 (10), 14173-14180.
33. Bailey, A. D.; Zhang, G.; Murphy, B. P., Comparison of damage to human hair fibers caused by monoethanolamine- and ammonia-based hair colorants. J Cosmet Sci 2014, 65 (1), 1-9.
34. Herrling, T.; Jung, K.; Fuchs, J., The role of melanin as protector against free radicals in skin and its role as free radical indicator in hair. Spectrochim Acta A 2008, 69 (5), 1429-1435.
35. Jablonski, N. G.; Chaplin, G., Human skin pigmentation as an adaptation to UV radiation. P Natl Acad Sci USA 2010, 107, 8962-8968.
36. Brenner, M.; Hearing, V. J., The protective role of melanin against UV damage in human skin. Photochem Photobiol 2008, 84 (3), 539-549.
37. Draelos, Z. D., Sunscreens and hair photoprotection. Dermatologic Clinics 2006, 24 (1), 81-+.
38. Nogueira, A. C. S.; Richena, M.; Dicello, L. E.; Joekes, I., Photo yellowing of human hair. J Photoch Photobio B 2007, 88 (2-3), 119-125.
39. Young, A. R.; Claveau, J.; Rossi, A. B., Ultraviolet radiation and the skin: Photobiology and sunscreen photoprotection. J Am Acad Dermatol 2017, 76 (3), S100-S109.
40. Kim, J. Y.; Kim, W. I.; Youn, W.; Seo, J.; Kim, B. J.; Lee, J. K.; Choi, I. S., Enzymatic film formation of nature-derived phenolic amines. Nanoscale 2018, 10 (28), 13351-13355.
41. Ni, QZ.; Sierra, B. N.; La Clair J. J., Burkart M. D. Chemoenzymatic elaboration of the Raper-Mason pathway unravels the structural diversity within eumelanin pigments. Chem Sci2020, 11, 7836-7841.
42. Zaidi, K. U.; Ali, A. S.; Ali, S. A., Purification and characterization of melanogenic enzyme tyrosinase from button mushroom. Enzyme Res 2014, 2014, 120739.
43. Nogueira, A. C. S.; Dicelio, L. E.; Joekes, I., About photo-damage of human hair. Photoch Photobio Sci 2006, 5 (2), 165-169.
44. Santos Nogueira, A. C.; Joekes, I., Hair color changes and protein damage caused by ultraviolet radiation. J Photochem Photobiol B 2004, 74 (2-3), 109-17.
45. Lee, W. S., Photoaggravation of hair aging. Int J Trichology 2009, 1 (2), 94-9.
46. Richena, M.; Rezende, C. A., Morphological degradation of human hair cuticle due to simulated sunlight irradiation and washing. J Photoch Photobio B 2016, 161, 430-440.
47. Cao, Y.; Qu, H.; Xiong, C.; Liu, C. H.; Zheng, L., A novel method for non-destructive determination of hair photo-induced damage based on multispectral imaging technology. Sci Rep-Uk 2017, 7.
48. Ji, J. H.; Park, T. S.; Lee, H. J.; Kim, Y. D.; Pi, L. Q.; Jin, X. H.; Lee, W. S., The Ethnic Differences of the Damage of Hair and Integral Hair Lipid after Ultra Violet Radiation. Annals of Dermatology 2013, 25 (1), 54-60.
49. Richena, M.; Rezende, C. A., Effect of photodamage on the outermost cuticle layer of human hair. J Photoch Photobio B 2015, 153, 296-304.
50. Robbins, C. R., Bleaching and Oxidation of Human Hair. In Chemical and Physical Behavior of Human Hair, Springer Berlin Heidelberg: Berlin, Heidelberg, 2012; pp 263-328.
51. Maia, R.; Gruson, H.; Endler, J. A.; White, T. E., pavo 2: New tools for the spectral and spatial analysis of colour in r. Methods Ecol Evol 2019, 10 (7), 1097-1107.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every material, nanoparticle, dispersion, molecule, formulation, combination of components, or method described or exemplified herein can be used to practice the invention, unless otherwise stated.

Every device, system, formulation, plurality of nanoparticles, combination of components, or methods described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

The term “and/or” is used herein, in the description and in the claims, to refer to a single element alone or any combination of elements from the list in which the term and/or appears. In other words, a listing of two or more elements having the term “and/or” is intended to cover embodiments having any of the individual elements alone or having any combination of the listed elements. For example, the phrase “element A and/or element B” is intended to cover embodiments having element A alone, having element B alone, or having both elements A and B taken together. For example, the phrase “element A, element B, and/or element C” is intended to cover embodiments having element A alone, having element B alone, having element C alone, having elements A and B taken together, having elements A and C taken together, having elements B and C taken together, or having elements A, B, and C taken together.

The term “±” refers to an inclusive range of values, such that “X±Y,” wherein each of X and Y is independently a number, refers to an inclusive range of values selected from the range of X−Y to X+Y.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method of treating hair of a subject with an artificial melanin material, the method comprising:
contacting in a solution artificial melanin precursors with an oxidizing agent at a temperature greater than or equal to 30° C. in the presence of the hair of said subject to form said artificial melanin material;
wherein said artificial melanin material associates with said hair of said subject, thereby treating the hair of said subject.

2. The method of claim 1, wherein said contacting step results in deposition of said artificial melanin material on the hair of said subject and/or uptake of said artificial melanin material into the hair of said subject.

3. The method of any one of claims 1-2, wherein said contacting step results in covalent or noncovalent association of said artificial melanin material with the hair of said subject.

4. The method of any of claims 1-3, wherein said contacting step results in a change in the color of the hair of said subject.

5. The method of claim 4, wherein said change in the color of the hair of said subject remains persistent for at least a period of 5 weeks.

6. The method of claim 4, wherein said change in the color of the hair of said subject remains persistent for at least 18 washing or rinsing cycles for said hair.

7. The method of any one of the preceding claims, wherein said contacting step is carried out for a time selected from the range of 1 hour to 5 hours.

8. The method of any one of the preceding claims, wherein said contacting step is carried out in the presence of a metal chelating agent at a concentration of less than or equal to 15 mM.

9. The method of any one of the preceding claims, wherein said contacting step is carried out in the absence of an iron-containing chelating agent and/or a copper-containing chelating agent.

10. The method of any one of the preceding claims, wherein said contacting step is carried out in the absence of a metal chelating agent.

11. The method of any one of the preceding claims, wherein said contacting step is carried out in the absence of a strong oxidizing condition.

12. The method of any one of the preceding claims, wherein said contacting step is carried out in the absence of sodium periodate.

13. The method any one of the preceding claims, wherein said contacting step takes place under conditions providing for polymerization of said artificial melanin precursors to generate said artificial melanin material.

14. The method of any one of the preceding claims, wherein said contacting step is carried out at said temperature ranging from 30° C. to 45° C.

15. The method of any one of the preceding claims, wherein said contacting step is carried out at physiological temperature.

16. The method of one of the preceding claims, wherein the contacting step is carried out at a pH selected from the range of 4 to 10.

17. The method of one of the preceding claims, wherein the contacting step is carried out at a pH selected from the range of 6.5 to 7.5.

18. The method of any one of the preceding claims, wherein said contacting step is carried out at pH greater than 7.

19. The method of any one of the preceding claims, wherein said solution comprises an ammonia solution, or a sodium hydroxide solution.

20. The method of any one of the preceding claims, wherein said solution comprises monoethanolamine or a derivative thereof.

21. The method of any one of the preceding claims, wherein said solution comprises an alkaline buffer solution.

22. The method of claim 20, wherein the buffer solution comprises a tris(hydroxymethyl)aminomethane buffer solution.

23. The method of any one of the preceding claims, wherein said solution comprises ammonia at a concentration less than or equal to 10% (w/v).

24. The method of any one of the preceding claims, wherein said solution comprises ammonia at a concentration selected over the range of 1 to 6% (w/v).

25. The method of any one of the preceding claims, wherein said solution comprises sodium hydroxide at a concentration less than or equal to 0.1 N.

26. The method of any one of the preceding claims, wherein said solution comprises sodium hydroxide at a concentration selected over the range of 0.01 N to 0.1 N.

27. The method of any one of the preceding claims, wherein said solution comprises tris(hydroxymethyl)aminomethane at a concentration less than or equal to 50 mM.

28. The method of any one of the preceding claims, wherein said solution comprises tris(hydroxymethyl)aminomethane at a concentration selected over the range of 1 mM to 50 mM. (SOI selected over the range of 10 mM to 50 mM.

29. The method of any one of the preceding claims, wherein said solution is an enzyme-containing solution comprising one or more enzymes.

30. The method of claim 29, wherein the one or more enzymes comprises one or more oxidoreductase enzymes.

31. The method of claim 29, wherein the one or more enzymes comprises tyrosinase and/or laccase.

32. The method of any one of claims 29-31, wherein the enzyme-containing solution comprises the one or more enzymes at a concentration selected from the range of 1 to 1000 U/mL.

33. The method of any one of the preceding claims, wherein said solution comprises monoethanolamine or a derivative thereof at a concentration less than or equal to 6% (w/v).

34. The method of any one of the preceding claims, wherein said solution comprises monoethanolamine or a derivative thereof at a concentration selected over the range of 1% to 3% (w/v).

35. The method of any one of the preceding claims, wherein the concentration of the artificial melanin precursors is greater than 0.1 mg mL⁻¹.

36. The method of any one of the preceding claims, wherein the concentration of the artificial melanin precursors is selected over the range of 0.1 mg mL⁻¹ to a saturated solution.

37. The method of any one of the preceding claims, wherein said oxidizing agent is O₂.

38. The method of any one of the preceding claims, wherein said oxidizing agent is present in the ambient atmosphere and wherein the solution is exposed to the ambient atmosphere during the contacting step.

39. The method of claim 37, wherein said O₂ is from air in contact with said solution.

40. The method of claim 37, wherein said O₂ is from air in contact with said alkaline solution or said enzyme-containing solution.

41. The method of any of claims 37-40, wherein the concentration of O₂ in said solution is greater than 0 and up to a saturated solution.

42. The method of any one of the preceding claims, wherein said contacting step is further carried out in the presence of H₂O₂ provided in said alkaline solution.

43. The method of claim 42, wherein the concentration of said H₂O₂ is less than or equal to 6% (w/v).

44. The method of claim 42, wherein the concentration of said H₂O₂ is selected over the range of 0.01% to 6% (w/v).

45. The method of any one of the preceding claims, wherein said contacting step is carried out in the absence of one or more metal salts.

46. The method of any one of the preceding claims, wherein said contacting step is further carried out in the presence of one or more metal salts and H₂O₂ provided in said alkaline solution.

47. The method of claim 46 wherein said one or more metal salts are nontoxic.

48. The method of any one of claims 46-47, wherein the one or more metal salts comprise one or more iron salts and/or one or more copper salts.

49. The method of any one of claims 46-48, wherein a concentration of the one or more metals salts in the solution is less than 15 mM.

50. The method of any one of claims 46-49, wherein the metal salt is CuSO₄.

51. The method of claim 50, wherein the concentration of said CuSO₄ is less than or equal to 15 mM.

52. The method of claim 50 or 51, wherein the concentration of said CuSO₄ is selected over the range of 1 mM to 15 mM.

53. The method of any of claims 42-52, wherein the concentration of said H₂O₂ is selected over the range of 0.01% to 6% (w/v).

54. The method of any one of the preceding claims, wherein the artificial melanin precursors are substituted or unsubstituted catechol-based or polyol-based compounds.

55. The method of any one of the preceding claims, wherein the artificial melanin precursors are substituted or unsubstituted: dopamine monomers, 1,8-Dihydroxynaphthalene or its derivative, tyrosine monomers, tyramine monomers, amino acids, phenolamines, catecholamines, or any combination of these.

56. The method of any one of the preceding claims, wherein the artificial melanin precursors are substituted or unsubstituted: dopamine monomers, tyrosine monomers, tyramine monomers, or a combination of these.

57. The method of any one of the preceding claims, wherein the artificial melanin precursors are free of phenol derivatives, resorcinol, and/or paraphenylenediamine.

58. The method of any one of the preceding claims, wherein the dopamine monomers are selected from the group consisting of substituted or unsubstituted: dihydroxydopamine monomers, dihydroxydopamine dimers, dihydroxydopamine oligomers, dioxydopamine monomers, dioxydopamine dimers, dioxydopamine oligomers, dihydroxynapthalene monomers, dihydroxynapthalene dimers, dihydroxynapthalene oligomers, dioxydopamine monomers, dioxydopamine dimers, dioxydopamine oligomers, and any combination of these.

59. The method of any one of the preceding claims, wherein the dopamine monomers are selected from the group consisting of tyrosine and derivatives, phenol and derivatives, resorcinol and derivatives, and any combinations thereof.

60. The method of any one of the preceding claims, wherein the dopamine monomers are selected from the group consisting of cysteine derivatives, chalcogenides derivatives, selenocysteine, and any combinations thereof.

61. The method of any one of the preceding claims, wherein the artificial melanin precursors are one or more monomers selected from the group consisting of: 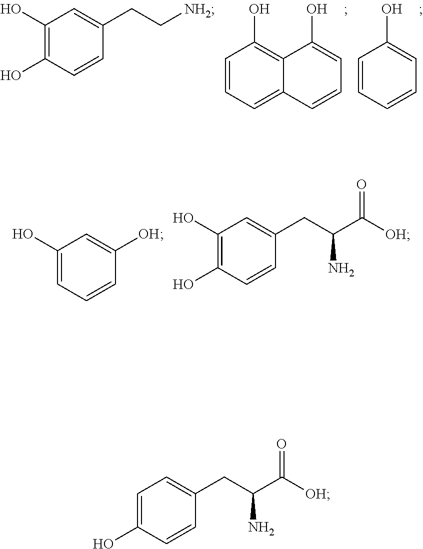
any combinations thereof, and any derivatives thereof.

62. The method of any one of the preceding claims, wherein at least a portion of the artificial melanin precursors have one or more thiol-reactive moieties.

63. The method of claim 62, wherein said thiol-reactive moieties are one or more groups selected from the group consisting of a thiol, maleimide, pyridyl disulfide-based compound, alkene, alkyl halide and any combinations thereof.

64. The method of any one of the preceding claims, wherein the artificial melanin material comprises a polymerization product of said artificial melanin precursors.

65. The method of any one of the preceding claims, wherein the artificial melanin material comprises artificial melanin nanoparticles, artificial melanin films, artificial melanin flakes, or any combination of these.

66. The method of any one of the preceding claims, wherein the artificial melanin material comprises artificial melanin nanoparticles.

67. The method of claim 66, wherein the melanin nanoparticles form a coating on said hair.

68. The method of claim 67, wherein said coating is characterized by nanostructures have size domains ranging from 5 nm to 500 nm.

69. The method of claim 66, wherein the melanin nanoparticles form a film on said hair.

70. The method of any one of the preceding claims, wherein the temperature is 35 to 45° C. and wherein:
a. said hair is originally a blond color, said alkaline solution is 1% to 6% (w/v) ammonia solution, wherein upon said contacting step said hair changes to a dark brown color; or
b. said hair is originally a blond color, said alkaline solution is 0.01-0.1 N sodium hydroxide solution, wherein upon said contacting step said hair changes to a dark brown color; or
c. said hair is originally a blond color, wherein said contacting step is further carried out in the presence of CuSO₄ having a concentration selected over the range of 1 mM to 15 mM and H₂O₂ having a concentration of 0.025% to 0.07% (w/v), wherein upon said contacting step said hair changes to a dark brown color; or
d. said hair is originally a blond color, said alkaline solution is 1 to 50 mM tris(hydroxymethyl)aminomethane buffer solution, wherein upon said contacting step said hair changes to a dark grey or light brown color; or
e. said hair is originally a blond color, wherein said contacting step is further carried out in the presence of CuSO₄ having a concentration selected over the range of 1 mM to 15 mM and H₂O₂ having a concentration of 0.025% to 0.07% (w/v), wherein upon said contacting step said hair changes to a dark brown color; or
f. said hair is originally a blond color, wherein said contacting step is further carried out in the presence of CuSO₄ having a concentration selected over the range of 1 mM to 15 mM and H₂O₂ having a concentration of 0.1% to 0.2% (w/v), wherein upon said contacting step said hair changes to a dark brown color; or
g. said hair is originally a blond color, wherein said contacting step is further carried out in the presence of CuSO₄ having a concentration selected over the range of 1 mM to 15 mM and H₂O₂ having a concentration of 0.2% to 0.4% (w/v), wherein upon said contacting step said hair changes to a brown color with shades of red; or
h. said hair is originally a blond color, wherein said contacting step is further carried out in the presence of CuSO₄ having a concentration selected over the range of 1 mM to 15 mM and H₂O₂ having a concentration of 2% to 4% (w/v), wherein upon said contacting step said hair changes to an orange color;
i. said hair is originally a blond color, said alkaline solution is 1% to 4% (w/v) ammonia solution and wherein said contacting step is further carried out in the presence of H₂O₂ having a concentration of 0.1% to 0.2% (w/v), wherein upon said contacting step said hair changes to a brown color; or
j. said hair is originally a blond color, said alkaline solution is 1% to 4% (w/v) ammonia solution and wherein said contacting step is further carried out in the presence of H₂O₂ having a concentration of 0.2% to 0.4% (w/v), wherein upon said contacting step said hair changes to a brown color with orange shades; or
k. said hair is originally a blond color, said alkaline solution is 1% to 4% (w/v) ammonia solution and wherein said contacting step is further carried out in the presence of H₂O₂ having a concentration of 2% to 4% (w/v), wherein upon said contacting step said hair changes to a bright blond color; or
l. said hair is originally a brown color, said alkaline solution is 1% to 3% (w/v) ammonia solution, wherein upon said contacting step said hair changes to a dark brown color; or
m. said hair is originally a red color, said alkaline solution is 1% to 3% (w/v) ammonia solution, wherein upon said contacting step said hair changes to a dark brown color with shades of red; or
n. said hair is originally a grey color, said alkaline solution is 1% to 3% (w/v) ammonia solution, wherein upon said contacting step said hair changes to a dark brown color; or
o. said hair is originally a grey color, said alkaline solution is 1% to 3% by wt ammonia solution and wherein said contacting step is further carried out in the presence of H₂O₂ having a concentration of 0.1% to 0.2% (w/v), wherein upon said contacting step said hair changes to a dark brown color with shades of red.

71. A method of treating hair of a subject with an artificial melanin material, the method comprising:
contacting in an alkaline solution artificial melanin precursors with an oxidizing agent at a temperature greater than or equal to 30° C. in the presence of the hair of said subject to form said artificial melanin material;
wherein at least a portion of the artificial melanin precursors have one or more thiol-reactive moieties; and
wherein said artificial melanin material associates with said hair of said subject, thereby treating the hair of said subject.

72. A method of treating hair of a subject with an artificial melanin material, the method comprising:
contacting in an alkaline solution the hair of said subject with the artificial melanin material having one or more thiol-reactive moieties;
wherein said artificial melanin material associates with said hair of said subject, thereby treating the hair of said subject.

73. The method of claim 71 or 72, wherein said one or more thiol-reactive moieties are one or more groups selected from the group consisting of a thiol, maleimide, pyridyl disulfide-based compound, alkene, alkyl halide and any combinations thereof

74. The method of any one of the preceding claims, wherein said contacting step is carried out in the absence of a metal chelating agent.

75. The method of any one of the preceding claims, wherein said contacting step is carried out in the absence of a strong oxidizing condition.

76. The method of any one of the preceding claims, wherein said contacting step is carried out in the absence of sodium periodate.

77. The method of any one of the preceding claims, wherein said contacting step does not change the mechanical properties of the hair of said subject.

78. The method of any one of claims 72-77, wherein the artificial melanin material comprises artificial melanin nanoparticles.

79. The method of any one of claims 72-78 wherein the solution is free of artificial melanin precursors or monomers.

80. A method of treating hair of a subject with an artificial melanin material, the method comprising:
contacting in an enzyme-containing solution the hair of said subject with the artificial melanin material having one or more thiol-reactive moieties; wherein the enzyme-containing solution comprises one or more enzymes; and
wherein said artificial melanin material associates with said hair of said subject, thereby treating the hair of said subject.

81. The method of claim 80, wherein said one or more thiol-reactive moieties are one or more groups selected from the group consisting of a thiol, maleimide, pyridyl disulfide-based compound, alkene, alkyl halide and any combinations thereof

82. The method of any one of claims 80-81, wherein the one or more enzymes comprises one or more oxidoreductase enzymes.

83. The method of any one of claims 80-81, wherein the one or more enzymes comprises tyrosinase and/or laccase.

84. The method of any of claims 80-83, wherein said contacting step is carried out in the absence of a metal chelating agent.

85. The method of any of claims 80-84, wherein said contacting step is carried out in the absence of a strong oxidizing condition.

86. The method of any of claims 80-85, wherein said contacting step is carried out in the absence of sodium periodate.

87. The method of any of claims 80-86, wherein said contacting step does not change the mechanical properties of the hair of said subject.

88. The method of any one of claims 80-87 wherein the artificial melanin material comprises artificial melanin nanoparticles.

89. The method of any one of claims 80-88 wherein the solution is free of artificial melanin precursors or monomers.

90. The method of any one of claims 80-89 wherein the solution has a pH selected from the range of 4 to 10.

91. A composition of matter comprising the hair of said subject treated with said artificial melanin material generated by any of the methods according to any one of the preceding claims.

92. A composition of matter comprising hair of a subject having a coating of artificial melanin nanoparticles, wherein said coating of artificial melanin nanoparticles is characterized by nanostructures have size domains ranging from 5 nm to 500 nm; wherein said artificial melanin nanoparticles associate with said hair of said subject.

93. An artificial melanin material comprising a polymerization product of artificial melanin precursors at least a portion of which having one or more thiol-reactive moieties.

94. An artificial melanin material produced by a method comprising:
contacting in an alkaline solution artificial melanin precursors with an oxidizing agent at a temperature greater than or equal to 18° C. in the presence of the hair of said subject to form said artificial melanin material;
wherein said artificial melanin material associates with said hair of said subject, thereby treating the hair of said subject.

95. An artificial melanin material produced by a method comprising:
contacting in an alkaline solution the hair of said subject with the artificial melanin material having thiol-reactive moieties;
wherein said artificial melanin material associates with said hair of said subject, thereby treating the hair of said subject.

96. The artificial melanin material of claim 94 or 95, wherein said thiol-reactive moieties are one or more groups selected from the group consisting of a thiol, maleimide, pyridyl disulfide-based compound, alkene, alkyl halide and any combinations thereof.

97. A method of treating hair of a subject with an artificial melanin material, the method comprising:
contacting in a solution artificial melanin precursors with an oxidizing agent at a temperature greater than or equal to 18° C. in the presence of the hair of said subject to form said artificial melanin material;
wherein said artificial melanin material associates with said hair of said subject, thereby treating the hair of said subject; wherein:
a. said hair is originally a blond color, wherein said contacting step is further carried out in the presence of CuSO₄ having a concentration selected over the range of 1 mM to 15 mM and H₂O₂ having a concentration of 0.025% to 0.07% by weight, wherein upon said contacting step said hair changes to a dark brown color; or
b. said hair is originally a blond color, wherein said contacting step is further carried out in the presence of CuSO₄ having a concentration selected over the range of 1 mM to 15 mM and H₂O₂ having a concentration of 0.1% to 0.2% by weight, wherein upon said contacting step said hair changes to a dark brown color; or
c. said hair is originally a blond color, wherein said contacting step is further carried out in the presence of CuSO₄ having a concentration selected over the range of 1 mM to 15 mM and H₂O₂ having a concentration of 0.2% to 0.4% by weight, wherein upon said contacting step said hair changes to a brown color with shades of red; or
d. said hair is originally a blond color, wherein said contacting step is further carried out in the presence of CuSO₄ having a concentration selected over the range of 1 mM to 15 mM and H₂O₂ having a concentration of 2% to 4% by weight, wherein upon said contacting step said hair changes to a dark orange color;
e. said hair is originally a blond color, said alkaline solution is 1% to 4% by wt ammonia solution and wherein said contacting step is further carried out in the presence of H₂O₂ having a concentration of 0.1% to 0.2% by weight, wherein upon said contacting step said hair changes to a brown color; or
f. said hair is originally a blond color, said alkaline solution is 1% to 4% by wt ammonia solution and wherein said contacting step is further carried out in the presence of H₂O₂ having a concentration of 0.2% to 0.4% by weight, wherein upon said contacting step said hair changes to a brown color with orange shades; or
g. said hair is originally a blond color, said alkaline solution is 1% to 4% by wt ammonia solution and wherein said contacting step is further carried out in the presence of H₂O₂ having a concentration of 2% to 4% by weight, wherein upon said contacting step said hair changes to a bright blond color.

98. The method, composition or material of any of the preceding claims for changing the color of the hair of said subject.

99. The method, composition or material of any of the preceding claims for darkening the color of the hair of said subject.

100. The method, composition or material of any of the preceding claims for restoring the color of the hair of said subject to its natural color.

101. The method, composition or material of any of the preceding claims for the coloring of the eyebrows of said subject.

102. The method of any of the preceding claims, wherein the artificial melanin precursors are contacted in a solution with an oxidizing agent in the presence of the hair at room temperature and then the temperature is subsequently raised to a temperature equal to or greater than 30° C. to provide for treatment of the hair.

103. The method of any of the preceding claims, wherein the artificial melanin precursors and oxidizing agent are contacted in solution in the presence of the hair at a temperature equal to or greater than 30° C. to provide for treatment of the hair.
